SEQUENCE LISTING (59 sequences)

SEQ ID NO: 1 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 2 - protein, 918 aa, Homo sapiens
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 3 - protein, 422 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 4 - DNA, 19 bp, synthetic construct
  misc_feature 1..19: note = siRNA target IL-11
  source 1..19: mol_type = other DNA
ccttccaaagccagatctt

SEQ ID NO: 5 - DNA, 19 bp, synthetic construct
  misc_feature 1..19: note = siRNA target IL-11
  source 1..19: mol_type = other DNA
gcctgggcaggaacatata

SEQ ID NO: 6 - DNA, 19 bp, synthetic construct
  misc_feature 1..19: note = siRNA target IL-11
  source 1..19: mol_type = other DNA
cctgggcaggaacatatat

SEQ ID NO: 7 - DNA, 19 bp, synthetic construct
  misc_feature 1..19: note = siRNA target IL-11
  source 1..19: mol_type = other DNA
ggttcattatggctgtgtt

SEQ ID NO: 8 - DNA, 19 bp, synthetic construct
  misc_feature 1..19: note = siRNA target IL-11Ralpha
  source 1..19: mol_type = other DNA
ggaccataccaaaggagat

SEQ ID NO: 9 - DNA, 19 bp, synthetic construct
  misc_feature 1..19: note = siRNA target IL-11Ralpha
  source 1..19: mol_type = other DNA
gcgtctttgggaatccttt

SEQ ID NO: 10 - DNA, 18 bp, synthetic construct
  misc_feature 1..18: note = siRNA target IL-11Ralpha
  source 1..18: mol_type = other DNA
gcaggacagtagatccct

SEQ ID NO: 11 - DNA, 19 bp, synthetic construct
  misc_feature 1..19: note = siRNA target IL-11Ralpha
  source 1..19: mol_type = other DNA
gctcaaggaacgtgtgtaa

SEQ ID NO: 12 - DNA, 42 bp, synthetic construct
  misc_feature 1..42: note = siRNA to IL-11 (NM_000641.3)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(3..4,16,18..19,26,28,32..34): mod_base = OTHER; note = uracil
ccttccaaagccagatcttnnaagatctggctttggaaggnn

SEQ ID NO: 13 - DNA, 42 bp, synthetic construct
  misc_feature 1..42: note = siRNA to IL-11 (NM_000641.3)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(4,16,18,22,24,26,28..29,32): mod_base = OTHER; note = uracil
gcctgggcaggaacatatanntatatgttcctgcccaggcnn

SEQ ID NO: 14 - DNA, 42 bp, synthetic construct
  misc_feature 1..42: note = siRNA to IL-11 (NM_000641.3)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(3,15,17,19,23,25,27,29..30,33): mod_base = OTHER; note = uracil
cctgggcaggaacatatatnnatatatgttcctgcccaggnn

SEQ ID NO: 15 - DNA, 42 bp, synthetic construct
  misc_feature 1..42: note = siRNA to IL-11 (NM_000641.3)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(3..4,7..8,10,14,16,18..19,32,35): mod_base = OTHER; note = uracil
ggttcattatggctgtgttnnaacacagccataatgaaccnn

SEQ ID NO: 16 - DNA, 42 bp, synthetic construct
  misc_feature 1..42: note = siRNA to IL-11Ralpha (U32324.1)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(7,19,23,25,28..30,33,35,38): mod_base = OTHER; note = uracil
ggaccataccaaaggagatnnatctcctttggtatggtccnn

SEQ ID NO: 17 - DNA, 42 bp, synthetic construct
  misc_feature 1..42: note = siRNA to IL-11Ralpha (U32324.1)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(4,6..8,14,17..19,28..29): mod_base = OTHER; note = uracil
gcgtctttgggaatcctttnnaaaggattcccaaagacgcnn

SEQ ID NO: 18 - DNA, 42 bp, synthetic construct
  misc_feature 1..42: note = siRNA to IL-11Ralpha (U32324.1)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(10,14,18,22,28,30,33,35,38): mod_base = OTHER; note = uracil
gcaggacagtagatccctanntagggatctactgtcctgcnn

SEQ ID NO: 19 - DNA, 42 bp, synthetic construct
  misc_feature 1..42: note = siRNA to IL-11Ralpha (U32324.1)
  modified_base 20..21: mod_base = OTHER; note = n = deoxythymidine
  modified_base 41..42: mod_base = OTHER; note = n = deoxythymidine
  source 1..42: mol_type = other DNA
  modified_base order(3,13,15,17,22..23,31..32,35..36): mod_base = OTHER; note = uracil
gctcaaggaacgtgtgtaannttacacacgttccttgagcnn

SEQ ID NO: 20 - protein, 21 aa, synthetic construct
  REGION 1..21: note = 20 amino acid linker
GPAGQSGGGGGSGGGSGGGSV

SEQ ID NO: 21 - protein, 516 aa, synthetic construct
  REGION 1..516: note = Hyper IL-11 (IL-11RA:IL-11 fusion)
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGPAGQSGGGGGSGGGSGGGSVPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 22 - protein, 119 aa, synthetic construct
  REGION 1..119: note = Enx203 VH
EVQLQQSGPELVKPGASVKIPCKASGYTFTDYNMDWVKQSHGKSLEWIGDINPHNGGPIYNQKFTGKATLTVDKSSSTAYMELRSLTSEDTAVYYCARGELGHWYFDVWGTGTTVTVSS

SEQ ID NO: 23 - protein, 111 aa, synthetic construct
  REGION 1..111: note = Enx203 VL
DIVLTQSPASLAVSLGQRATISCRASKSVSTSGYSYIHWYQQKPGQPPKLLIYLASNLDSGVPARFSGSGSGTDFTLNIHPVEEEDAATYYCQHSRDLPPTFGGGTKLEIK

SEQ ID NO: 24 - protein, 117 aa, synthetic construct
  REGION 1..117: note = Enx209 VH
QVQLQQPGAELVRPGSSVKLSCKASGYTFTNYWMHWLKQRPVQGLEWIGNIGPSDSKTHYNQKFKDKATLTVDKSSSTAYMQLNSLTSEDSAVYYCARGDYVLFTYWGQGTLVTVSA

SEQ ID NO: 25 - protein, 107 aa, synthetic construct
  REGION 1..107: note = Enx209 VL
DIVLTQSPATLSLSPGERATLSCRASQSISNNLHWYQQKSHEAPRLLIKYASQSISGIPARFSGSGSGTDFTLSFSSLETEDFAVYFCQQSYSWPLTFGQGTKLEIK

SEQ ID NO: 26 - protein, 118 aa, synthetic construct
  REGION 1..118: note = Enx108A VH
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSS

SEQ ID NO: 27 - protein, 111 aa, synthetic construct
  REGION 1..111: note = Enx108A VL
QSALTQPRSVSGSPGQSVTLSCTGTSSDVGGYNYVSWYQHYPGKAPKLMIFDVNERSSGVPDRFSGSKSGNTASLTISGLQAEDEADYYCASYAGRYTWMFGGGTKVTVLG

SEQ ID NO: 28 - protein, 445 aa, synthetic construct
  REGION 1..445: note = Enx108A hIgG4 (L248E, S241P) HC
QVQLVQSGGGVVQPGRSLRLSCAASGFTFSSYGMHWVRQAPGKGLEWVAVISYDGSNKYYADSVKGRFTISRDNSKNTLYLQMNSLRAEDTAVYYCAKIGATDPLDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 216 aa, synthetic construct
  REGION 1..216: note = Enx108A lambda LC
QSALTQPRSVSGSPGQSVTLSCTGTSSDVGGYNYVSWYQHYPGKAPKLMIFDVNERSSGVPDRFSGSKSGNTASLTISGLQAEDEADYYCASYAGRYTWMFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 119 aa, synthetic construct
  REGION 1..119: note = hEnx203 VH
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSS

SEQ ID NO: 31 - protein, 111 aa, synthetic construct
  REGION 1..111: note = hEnx203 VL
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIK

SEQ ID NO: 32 - protein, 117 aa, synthetic construct
  REGION 1..117: note = hEnx209 VH
QVQLVQSGAEVKKPGASVKVSCKASGYTFTNYWMHWLRQRPGQGLEWIGNIGPSDSKTHYNQKFKDRVTMTVDKSTSTAYMELSSLRSEDTAVYYCARGDYVLFTYWGQGTLVTVSS

SEQ ID NO: 33 - protein, 107 aa, synthetic construct
  REGION 1..107: note = hEnx209 VL
DIVLTQSPATLSLSPGERATLSCRASQSISNNLHWYQQKPGQAPRLLIKYASQSISGIPARFSGSGSGTDFTLTISSLEPEDFAVYYCQQSYSWPLTFGQGTKLEIK

SEQ ID NO: 34 - protein, 5 aa, synthetic construct
  REGION 1..5: note = Enx108A VH CDR1
SYGMH

SEQ ID NO: 35 - protein, 17 aa, synthetic construct
  REGION 1..17: note = Enx108A VH CDR2
VISYDGSNKYYADSVKG

SEQ ID NO: 36 - protein, 9 aa, synthetic construct
  REGION 1..9: note = Enx108A VH CDR3
IGATDPLDY

SEQ ID NO: 37 - protein, 14 aa, synthetic construct
  REGION 1..14: note = Enx108A VL CDR1
TGTSSDVGGYNYVS

SEQ ID NO: 38 - protein, 7 aa, synthetic construct
  REGION 1..7: note = Enx108A VL CDR2
DVNERSS

SEQ ID NO: 39 - protein, 10 aa, synthetic construct
  REGION 1..10: note = Enx108A VL CDR3
ASYAGRYTWM

SEQ ID NO: 40 - protein, 5 aa, synthetic construct
  REGION 1..5: note = Enx203, hEnx203 VH CDR1
DYNMD

SEQ ID NO: 41 - protein, 17 aa, synthetic construct
  REGION 1..17: note = Enx203, hEnx203 VH CDR2
DINPHNGGPIYNQKFTG

SEQ ID NO: 42 - protein, 10 aa, synthetic construct
  REGION 1..10: note = Enx203, hEnx203 VH CDR3
GELGHWYFDV

SEQ ID NO: 43 - protein, 15 aa, synthetic construct
  REGION 1..15: note = Enx203, hEnx203 VL CDR1
RASKSVSTSGYSYIH

SEQ ID NO: 44 - protein, 7 aa, synthetic construct
  REGION 1..7: note = Enx203, hEnx203 VL CDR2
LASNLDS

SEQ ID NO: 45 - protein, 9 aa, synthetic construct
  REGION 1..9: note = Enx203, hEnx203 VL CDR3
QHSRDLPPT

SEQ ID NO: 46 - protein, 5 aa, synthetic construct
  REGION 1..5: note = Enx209, hEnx209 VH CDR1
NYWMH

SEQ ID NO: 47 - protein, 17 aa, synthetic construct
  REGION 1..17: note = Enx209, hEnx209 VH CDR2
NIGPSDSKTHYNQKFKD

SEQ ID NO: 48 - protein, 8 aa, synthetic construct
  REGION 1..8: note = Enx209, hEnx209 VH CDR3
GDYVLFTY

SEQ ID NO: 49 - protein, 11 aa, synthetic construct
  REGION 1..11: note = Enx209, hEnx209 VL CDR1
RASQSISNNLH

SEQ ID NO: 50 - protein, 7 aa, synthetic construct
  REGION 1..7: note = Enx209, hEnx209 VL CDR2
YASQSIS

SEQ ID NO: 51 - protein, 9 aa, synthetic construct
  REGION 1..9: note = Enx209, hEnx209 VL CDR3
QQSYSWPLT

SEQ ID NO: 52 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 107 aa, Homo sapiens [sequence omitted]

SEQ ID NO: 55 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 56 - protein, 449 aa, synthetic construct
  REGION 1..449: note = hEnx203 hIgG1 HC
EVQLVQSGAEVKKPGASVKISCKASGYTFTDYNMDWVKQAPGQRLEWIGDINPHNGGPIYNQKFTGRATLTVDKSASTAYMELSSLRSEDTAVYYCARGELGHWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 218 aa, synthetic construct
  REGION 1..218: note = hEnx203 kappa LC
DIVLTQSPASLALSPGERATLSCRASKSVSTSGYSYIHWYQQKPGQAPRLLIYLASNLDSGVPARFSGSGSGTDFTLTISSLEEEDFATYYCQHSRDLPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 444 aa, synthetic construct
  REGION 1..444: note = hEnx209 hIgG4 (L248E, S241P) HC
QVQLVQSGAEVKKPGASVKVSCKASGYTFTNYWMHWLRQRPGQGLEWIGNIGPSDSKTHYNQKFKDRVTMTVDKSTSTAYMELSSLRSEDTAVYYCARGDYVLFTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 214 aa, synthetic construct
  REGION 1..214: note = hEnx209 kappa LC
DIVLTQSPATLSLSPGERATLSCRASQSISNNLHWYQQKPGQAPRLLIKYASQSISGIPARFSGSGSGTDFTLTISSLEPEDFAVYYCQQSYSWPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0270310 A1
(43) Pub. Date: Aug. 28, 2025

(54) TREATMENT OF KIDNEY INJURY

(71) Applicants: National University of Singapore, Singapore (SG); Singapore Health Services PTE LTD., Singapore (SG)

(72) Inventors: Stuart Alexander Cook, Singapore (SG); Sebastian Schaefer, Singapore (SG); Anissa Anindya Widjaja, Singapore (SG)

(73) Assignees: National University of Singapore, Singapore (SG); Singapore Health Services PTE LTD., Singapore (SG)

(21) Appl. No.: 19/028,642

(22) Filed: Jan. 17, 2025

Related U.S. Application Data
(62) Division of application No. 17/751,117, filed on May 23, 2022, now Pat. No. 12,234,284.
Application No. 17/751,117 is a division of application No. 16/798,101, filed on Feb. 21, 2020, now Pat. No. 11,339,216.

(30) Foreign Application Priority Data
Feb. 22, 2019 (GB) 1902419.9

(51) Int. Cl. C07K 16/24; A61P 13/12; C07K 14/715; C12N 15/113

(52) U.S. Cl. CPC C07K 16/244; A61P 13/12; C07K 14/7155; C12N 15/1136

(57) ABSTRACT

Methods of treating and preventing kidney injury through inhibiting interleukin 11 (IL-11)-mediated signalling are disclosed, as well as agents for use in such methods.

### FIELD OF THE INVENTION

This application claims priority from GB 1902419.9 filed 22 Feb. 2019, the contents and elements of which are herein incorporated by reference for all purposes.

The present invention relates to the diagnosis, treatment and prophylaxis of diseases and conditions associated with kidney injury, particularly although not exclusively acute kidney injury.

### BACKGROUND TO THE INVENTION

Acute kidney injury (AKI) refers to rapid onset damage to the kidney, notably the tubular epithelial cells. It is often chemically-driven, for example renal damage or injury caused by a medicine, chemical, contrast dye or herbal or dietary supplements. It may also be caused by ischaemia, mechanical or immune factors.

AKI is a common condition and affects up to 10% of patients in hospital (Silver, S. A. & Chertow, G. M. The Economic Consequences of Acute Kidney Injury. Nephron 137, 297-301 (2017). There is major mortality associated with AKI and mortality in patients requiring intensive care post AKI can be up to 50%. For those surviving AKI there are long-term risks of progressing to chronic kidney disease (CKD), end stage renal failure, renal replacement therapy or transplantation (Silver, S. A. & Chertow, G. M. The Economic Consequences of Acute Kidney Injury. Nephron 137; Mehta, R. L. et al. Acute Kidney Injury Network: report of an initiative to improve outcomes in acute kidney injury. Crit. Care 11; Zuk, A. et al. Overcoming Translational Barriers in Acute Kidney Injury: A Report from an NIDDK Workshop. Clin. J. Am. Soc. Nephrol. 13, 1113-1123 (2018)).

The chemotherapeutic agent cisplatin (dichlorodiamino platinum; SP-4-2)-diamminedichloroplatinum(II)) is widely used to treat a range of cancers including head and neck, breast, lung, testis, ovarian, brain, and bladder cancers. After a single dose of cisplatin (50-100 mg/m²) most patients develop a degree of AKI and up to 30% of patients will develop nephrotoxicity (Ozkok, A. & Edelstein, C. L. Pathophysiology of cisplatin-induced acute kidney injury. Biomed Res. Int. 2014, 967826 (2014)), as defined by an abrupt reduction in kidney function signified by an increase in serum creatinine (Mehta et al supra). In elderly patients with head and neck cancer up to 20% will progress from AKI to severe kidney dysfunction (Yao, X., Panichpisal, K., Kurtzman, N. & Nugent, K. Cisplatin nephrotoxicity: a review. Am. J. Med. Sci. 334, 115-124 (2007)). Cisplatin-induced AKI is dose limiting and therapy is often split into multiple doses over several weeks but this is still associated with nephrotoxicity. The severity of cisplatin-induced AKI is more severe in the presence of pre-existing conditions that include diabetes, hypertension, nephrotoxic drugs and old age.

The pathophysiology of AKI is largely defined by damage of the renal tubular epithelial cells (TECs) by toxins, chemicals, drugs, immune and mechanical factors or ischaemia. There are also vascular and immune components to AKI. In patients treated with cisplatin, the drug is present in the kidney at levels up to five times those in the plasma. In the kidney, cisplatin accumulates in TECs where it induces reactive oxygen species, depletes glutathione, causes mitochondrial dysfunction and causes cell death through apoptosis or necrosis (Ozkok et al, supra; Wang, S., Wei, Q., Dong, G. & Dong, Z. ERK-mediated suppression of cilia in cisplatin-induced tubular cell apoptosis and acute kidney injury. Biochim. Biophys. Acta 1832, 1582-1590 (2013); Jo, S.-K., Cho, W. Y., Sung, S. A., Kim, H. K. & Won, N. H. MEK inhibitor, U0126, attenuates cisplatin-induced renal injury by decreasing inflammation and apoptosis. Kidney Int. 67; Nowak, G. Protein Kinase C-α and ERK1/2 Mediate Mitochondrial Dysfunction, Decreases in Active Na+Transport, and Cisplatin-induced Apoptosis in Renal Cells. J. Biol. Chem. 277, 43377-43388 (2002)). During ischemia, TECs that have very high oxygen demands become oxygen deprived and undergo cell death due to apoptosis or necrosis.

Following AKI, kidney function can recover. This is widely recognised as driven by proliferation of remaining TECs, which have very large regenerative capacity (Yang, H.-C., Liu, S.-J. & Fogo, A. B. Kidney regeneration in mammals. Nephron Exp. Nephrol. 126, 50 (2014); Coelho, S., Cabral, G., Lopes, J. A. & Jacinto, A. Renal regeneration after acute kidney injury. Nephrology 23, 805-814 (2018); Chang-Panesso, M. & Humphreys, B. D. Cellular plasticity in kidney injury and repair. Nat. Rev. Nephrol. 13, 39-46 (2017)). When TEC proliferation is inadequate nephrotoxicity develops and over time chronic kidney disease can ensue and fibrosis may occur as a secondary phenomenon. In recent studies it has been shown that a critical determinant of TEC dysfunction following AKI is re-expression of the SNAIL gene (also known as SNA; SNAH; SNAIL; SLUGH2; SNAIL1) (Grande, M. T. et al. Snail1-induced partial epithelial-to-mesenchymal transition drives renal fibrosis in mice and can be targeted to reverse established disease. Nat. Med. 21, 989-997 (2015); Simon-Tillaux, N. & Hertig, A. Snail and kidney fibrosis. Nephrol. Dial. Transplant 32, 224-233 (2017); Lovisa, S. et al. Epithelial-to-mesenchymal transition induces cell cycle arrest and parenchymal damage in renal fibrosis. Nat. Med. 21, 998-1009 (2015). SNAIL is important for embryogenesis but is rarely expressed in adults other than in cancers where it causes epithelial to mesenchymal transition (EMT). In the kidney, the TGFβ1 gene can induce SNAIL in TECs and this is associated with impaired TEC function and proliferation (Lovisa, S. et al. Epithelial-to-mesenchymal transition induces cell cycle arrest and parenchymal damage in renal fibrosis. Nat. Med. 21, 998-1009 (2015)).

The cytokine interleukin 11 (IL-11) reportedly has a powerful protective effect against AKI following ischemia-reperfusion injury and inhibits apoptosis, necrosis and inflammation in AKI (Lee, H. T. et al. Interleukin-11 protects against renal ischemia and reperfusion injury. Am. J. Physiol. Renal Physiol. 303, F1216-24 (2012). IL-11 has also been shown to be upregulated in human TECs and mouse kidney by isoflurane and this is associated with a critical protective role for IL-11 against acute ischemic kidney injury (Ham, A. et al. Critical role of interleukin-11 in isoflurane-mediated protection against ischemic acute kidney injury in mice. Anesthesiology 119, 1389-1401 (2013)).

IL-11 treatment has also been reported to protect against acute nephrotoxic nephritis in rats and mice by reducing kidney inflammation and preventing kidney damage (Stangou, M. et al. Effect of IL-11 on glomerular expression of TGF-beta and extracellular matrix in nephrotoxic nephritis in Wistar Kyoto rats. J. Nephrol. 24, 106-111 (2011); Lai, P. C. et al. Interleukin-11 attenuates nephrotoxic nephritis in Wistar Kyoto rats. J. Am. Soc. Nephrol. 12, 2310-2320 (2001); Lai, P. C. et al. Interleukin-11 reduces renal injury and glomerular NF-kappa B activity in murine experimental glomerulonephritis. Nephron Exp. Nephrol. 101, e146-54 (2005)).

### SUMMARY OF THE INVENTION

In contrast to the reported protective role of IL-11 in kidney injury and damage, the present invention relates to the treatment and/or prevention of kidney injury and disorders, diseases or conditions associated with kidney injury through the inhibition of IL-11 signalling.

In one aspect of the present invention there is provided an agent capable of inhibiting interleukin 11 (IL-11)-mediated signalling for use in a method of treating or preventing kidney injury and/or a disorder, disease or condition associated with kidney injury.

In another aspect of the present invention, there is provided the use of an agent capable of inhibiting interleukin 11 (IL-11)-mediated signalling for use in the manufacture of a medicament for use in a method of treating or preventing kidney injury and/or a disorder, disease or condition associated with kidney injury.

In another aspect of the present invention, there is provided a method of treating or preventing kidney injury and/or a disorder, disease or condition associated with kidney injury, the method comprising administering to a subject in need of treatment a therapeutically effective amount of an agent capable of inhibiting interleukin 11 (IL-11)-mediated signalling.

The present invention also provides an agent capable of inhibiting interleukin 11 (IL-11)-mediated signalling for use in a method of reversing kidney injury.

Also provided is the use of an agent capable of inhibiting interleukin 11 (IL-11)-mediated signalling for use in the manufacture of a medicament for use in a method of reversing kidney injury.

Also provided is a method of reversing kidney injury, the method comprising administering to a subject in need of treatment a therapeutically effective amount of an agent capable of inhibiting interleukin 11 (IL-11)-mediated signalling.

In some embodiments, the kidney injury is acute kidney injury. In some embodiments, the kidney injury is nephrotoxicity. In some embodiments, the kidney injury is drug-induced kidney injury or ischemia-induced kidney injury. In some embodiments, the kidney injury is cisplatin-induced kidney injury or cisplatin-induced nephrotoxicity. In some embodiments, the kidney injury is characterised by damage to tubular epithelial cells (TECs).

The present invention also provides an agent capable of inhibiting interleukin 11 (IL-11)-mediated signalling for use in a method of improving renal function in a subject suffering from an impairment to renal function.

Also provided is the use of an agent capable of inhibiting interleukin 11 (IL-11)-mediated signalling for use in the manufacture of a medicament for use in a method of improving renal function in a subject suffering from an impairment to renal function.

Also provided is a method of improving renal function in a subject suffering from an impairment to renal function, the method comprising administering to a subject in need of treatment a therapeutically effective amount of an agent capable of inhibiting interleukin 11 (IL-11)-mediated signalling.

The present invention also provides an agent capable of inhibiting interleukin 11 (IL-11)-mediated signalling for use in a method of promoting the proliferation, survival and/or function of tubular epithelial cells (TECs), and/or the growth, maintenance and/or function of renal tissue (e.g. following kidney injury).

Also provided is the use of an agent capable of inhibiting interleukin 11 (IL-11)-mediated signalling for use in the manufacture of a medicament for use in a method of promoting the proliferation, survival and/or function of tubular epithelial cells (TECs), and/or the growth, maintenance and/or function of renal tissue (e.g. following kidney injury).

Also provided is a method of promoting the proliferation, survival and/or function of tubular epithelial cells (TECs), and/or the growth, maintenance and/or function of renal tissue (e.g. following kidney injury), the method comprising administering to a subject in need of treatment a therapeutically effective amount of an agent capable of inhibiting interleukin 11 (IL-11)-mediated signalling.

The present invention also provides an agent capable of inhibiting interleukin 11 (IL-11)-mediated signalling for use in a method of inhibiting SNAIL expression (e.g. following kidney injury).

Also provided is the use of an agent capable of inhibiting interleukin 11 (IL-11)-mediated signalling for use in the manufacture of a medicament for use in a method of inhibiting SNAIL expression (e.g. following kidney injury).

Also provided is a method of inhibiting SNAIL expression (e.g. following kidney injury), the method comprising administering to a subject in need of treatment a therapeutically effective amount of an agent capable of inhibiting interleukin 11 (IL-11)-mediated signalling.

The present invention also provides an agent capable of inhibiting interleukin 11 (IL-11)-mediated signalling for use in a method of inhibiting or reversing the transition of tubular epithelial cells (TECs) to a mesenchymal cell-like phenotype (e.g. following kidney injury).

Also provided is the use of an agent capable of inhibiting interleukin 11 (IL-11)-mediated signalling for use in the manufacture of a medicament for use in a method of inhibiting or reversing the transition of tubular epithelial cells (TECs) to a mesenchymal cell-like phenotype (e.g. following kidney injury).

Also provided is a method of inhibiting or reversing the transition of tubular epithelial cells (TECs) to a mesenchymal cell-like phenotype (e.g. following kidney injury), the method comprising administering to a subject in need of treatment a therapeutically effective amount of an agent capable of inhibiting interleukin 11 (IL-11)-mediated signalling.

In some embodiments in accordance with various aspects of the present invention, the agent is an agent capable of preventing or reducing the binding of interleukin 11 (IL-11) to a receptor for interleukin 11 (IL-11R).

In some embodiments, the agent is capable of binding to interleukin 11 (IL-11) or a receptor for interleukin 11 (IL-11R). In some embodiments, the agent is selected from the group consisting of: an antibody or an antigen-binding fragment thereof, a polypeptide, a peptide, a nucleic acid, an oligonucleotide, an aptamer or a small molecule. The agent may be an antibody or an antigen-binding fragment thereof. The agent may be a decoy receptor.

In some embodiments, the agent is an anti-IL-11 antibody antagonist of IL-11-mediated signalling, or an antigen-binding fragment thereof.

In some embodiments, the antibody or antigen-binding fragment comprises:
(i) a heavy chain variable (VH) region incorporating the following CDRs:
   HC-CDR1 having the amino acid sequence of SEQ ID NO:34
   HC-CDR2 having the amino acid sequence of SEQ ID NO:35
   HC-CDR3 having the amino acid sequence of SEQ ID NO:36; and
(ii) a light chain variable (VL) region incorporating the following CDRs:
   LC-CDR1 having the amino acid sequence of SEQ ID NO:37
   LC-CDR2 having the amino acid sequence of SEQ ID NO:38
   LC-CDR3 having the amino acid sequence of SEQ ID NO:39.

In some embodiments, the antibody or antigen-binding fragment comprises:
(i) a heavy chain variable (VH) region incorporating the following CDRs:
   HC-CDR1 having the amino acid sequence of SEQ ID NO:40
   HC-CDR2 having the amino acid sequence of SEQ ID NO:41
   HC-CDR3 having the amino acid sequence of SEQ ID NO:42; and
(ii) a light chain variable (VL) region incorporating the following CDRs:
   LC-CDR1 having the amino acid sequence of SEQ ID NO:43
   LC-CDR2 having the amino acid sequence of SEQ ID NO:44
   LC-CDR3 having the amino acid sequence of SEQ ID NO:45.

In some embodiments, the agent is an anti-IL-11Rα antibody antagonist of IL-11-mediated signalling, or an antigen-binding fragment thereof.

In some embodiments, the antibody or antigen-binding fragment comprises:
(i) a heavy chain variable (VH) region incorporating the following CDRs:
   HC-CDR1 having the amino acid sequence of SEQ ID NO:46
   HC-CDR2 having the amino acid sequence of SEQ ID NO:47
   HC-CDR3 having the amino acid sequence of SEQ ID NO:48; and
(ii) a light chain variable (VL) region incorporating the following CDRs:
   LC-CDR1 having the amino acid sequence of SEQ ID NO:49
   LC-CDR2 having the amino acid sequence of SEQ ID NO:50
   LC-CDR3 having the amino acid sequence of SEQ ID NO:51.

In some embodiments, the agent is a decoy receptor for IL-11. In some embodiments the decoy receptor for IL-11 comprises: (i) an amino acid sequence corresponding to the cytokine binding module of gp130 and (ii) an amino acid sequence corresponding to the cytokine binding module of IL-11Rα.

In some embodiments the agent is an IL-11 mutein. In some embodiments the IL-11 mutein is W147A.

In some embodiments, the agent is capable of preventing or reducing the expression of interleukin 11 (IL-11) or a receptor for interleukin 11 (IL-11R). The agent may be an oligonucleotide or a small molecule.

In some embodiments the agent is an antisense oligonucleotide capable of preventing or reducing the expression of IL-11. In some embodiments the antisense oligonucleotide capable of preventing or reducing the expression of IL-11 is siRNA targeted to IL11 comprising the sequence of SEQ ID NO:12, 13, 14 or 15. In some embodiments the agent is an antisense oligonucleotide capable of preventing or reducing the expression of IL-11Rα. In some embodiments the antisense oligonucleotide capable of preventing or reducing the expression of IL-11Rα is siRNA targeted to IL11RA comprising the sequence of SEQ ID NO:16, 17, 18 or 19.

In any embodiments provided herein, the interleukin 11 receptor may be or comprise IL-11Rα.

In any embodiments provided herein, the kidney injury may be one of acute kidney injury (AKI), nephrotoxicity, drug-induced kidney injury (DIKI), acute kidney failure, acute kidney disease, chronic kidney disease, kidney damage, tubular necrosis, acute tubular necrosis, and autoimmune kidney injury.

In any embodiments provided herein, the agent may be administered before, in conjunction with, or after the cause of the kidney injury, e.g. administration or consumption of a nephrotoxic medicine or exposure to a physical, mechanical, chemical or environmental source of kidney injury.

In some embodiments, the agents, uses and methods herein are provided for treating and/or preventing drug-induced kidney injury (DIKI). The DIKI may be intrinsic and/or idiosyncratic kidney injury. In any embodiments, the agents, uses and methods herein may be provided for treating and/or preventing cisplatin-induced kidney injury and/or cisplatin-induced nephrotoxicity.

In some embodiments, the agents, uses and methods herein may be provided for treating and/or preventing ischemia-induced kidney injury (IIKI) or ischemia-induced acute kidney injury.

In any embodiments provided herein, the disorder, disease or condition associated with kidney injury may be a disease, disorder or condition in which kidney injury is pathologically implicated. The pathology may include damage to tubular epithelial cells and/or the transition of TECs to a mesenchymal cell-like phenotype, which may be proximal and/or distal.

In any embodiments, the method comprises administering the agent to a subject in which expression of interleukin 11 (IL-11) or a receptor for IL-11 (IL-11R) may be upregulated.

In any embodiments, the method may comprise administering the agent to a subject in which expression of interleukin 11 (IL-11) or a receptor for interleukin 11 (IL-11R) has been determined to be upregulated.

In some embodiments the method comprises determining whether expression of interleukin 11 (IL-11) or a receptor for IL-11 (IL-11R) is upregulated in the subject and administering the agent to a subject in which expression of interleukin 11 (IL-11) or a receptor for IL-11 (IL-11R) is upregulated.

Also provided is a method of determining the suitability of a subject for the treatment or prevention of kidney injury and/or a disorder, disease or condition associated with kidney injury with an agent capable of inhibiting interleukin 11 (IL-11)-mediated signalling, the method comprising determining, optionally in vitro, whether interleukin 11 (IL-11) or a receptor for IL-11 (IL-11R) expression is upregulated in the subject.

Also provided is a method of selecting a subject for the treatment or prevention of kidney injury and/or a disorder, disease or condition associated with kidney injury with an agent capable of inhibiting interleukin 11 (IL-11)-mediated signalling, the method comprising determining, optionally in vitro, whether interleukin 11 (IL-11) or a receptor for IL-11 (IL-11R) expression is upregulated in the subject.

In one aspect there is provided a method of diagnosing kidney injury and/or a disorder, disease or condition associated with kidney injury, or a risk of developing kidney injury and/or a disorder, disease or condition associated with kidney injury in a subject, the method comprising determining, optionally in vitro, the upregulation of interleukin 11 (IL-11) or an receptor for IL-11 (IL-11R) in a sample obtained from the subject. In some embodiments, the method of diagnosing is a method of confirming a diagnosis of kidney injury and/or a disorder, disease or condition associated with kidney injury in a subject suspected of having kidney injury and/or a disorder, disease or condition associated with kidney injury. In some embodiments a method of diagnosing and/or a method of confirming a diagnosis comprises selecting the subject for treatment with an agent capable of inhibiting interleukin 11 (IL-11)-mediated signalling.

Also provided is a method of providing a prognosis for a subject having, or suspected of having, kidney injury and/or a disorder, disease or condition associated with kidney injury, the method comprising determining, optionally in vitro, whether expression of interleukin 11 (IL-11) or a receptor for IL-11 (IL-11R) is upregulated in a sample obtained from the subject and, based on the determination, providing a prognosis for treatment of the subject with an agent capable of inhibiting interleukin 11 (IL-11)-mediated signalling. In some embodiments, a method of providing a prognosis comprises selecting a subject determined to have upregulated expression of interleukin 11 (IL-11) or a receptor for IL-11 (IL-11R) for treatment with an agent capable of inhibiting interleukin 11 (IL-11)-mediated signalling.

In another aspect there is provided a method of diagnosing kidney injury and/or a disorder, disease or condition associated with kidney injury or a risk of developing kidney injury and/or a disorder, disease or condition associated with kidney injury, the method comprising determining, optionally in vitro, one or more genetic factors in the subject that are predictive of upregulation of expression of IL-11 or a receptor for IL-11, or of upregulation of IL-11 mediated signalling. In some embodiments the method comprises selecting the subject for treatment with an agent capable of inhibiting interleukin 11 (IL-11)-mediated signalling.

Also provided is a method of providing a prognosis for a subject having, or suspected of having, kidney injury and/or a disorder, disease or condition associated with kidney injury, the method comprising determining, optionally in vitro, one or more genetic factors in the subject that are predictive of upregulation of expression of IL-11 or a receptor for IL-11, or of upregulation of IL-11 mediated signalling.

### DESCRIPTION

There is ongoing demand for effective prevention and treatment of kidney injury, particularly acute kidney injury.

The cytokine IL-11 has been reported to have a protective effect against acute kidney injury following ischemia-reperfusion injury (Lee et al supra) and IL-11 has been reported to be upregulated and protective in human tubular epithelial cells and mouse kidney upon chemical damage by isoflurane (Ham et al supra).

In contrast, however, the present inventors have found that inhibition of IL-11 mediated signalling is effective to protect tubular epithelial cells, allowing them to proliferate and recover from damage that is a common causative factor of acute kidney injury. Whilst not wishing to be bound by theory, the inventors believe that inhibition of IL-11 enables tubular epithelial cells to proliferate leading to renal tissue regeneration and recovery by preventing or reducing the expression of SNAIL.

### Interleukin 11 and Receptors for IL-11

Interleukin 11 (IL-11), also known as adipogenesis inhibitory factor, is a pleiotropic cytokine and a member of the IL-6 family of cytokines that includes IL-6, IL-11, IL-27, IL-31, oncostatin, leukemia inhibitory factor (LIF), cardiotrophin-1 (CT-1), cardiotrophin-like cytokine (CLC), ciliary neurotrophic factor (CNTF) and neuropoetin (NP-1).

Interleukin 11 (IL-11) is expressed in a variety of mesenchymal cell types. IL-11 genomic sequences have been mapped onto chromosome 19 and the centromeric region of chromosome 7, and is transcribed with a canonical signal peptide that ensures efficient secretion from cells. The activator protein complex of IL-11, cJun/AP-1, located within its promoter sequence is critical for basal transcriptional regulation of IL-11 (Du and Williams., Blood 1997, Vol 89:3897-3908). The immature form of human IL-11 is a 199 amino acid polypeptide whereas the mature form of IL-11 encodes a protein of 178 amino acid residues (Garbers and Scheller., Biol. Chem. 2013; 394(9):1145-1161). The human IL-11 amino acid sequence is available under UniProt accession no. P20809 (P20809.1 GI:124294; SEQ ID NO:1). Recombinant human IL-11 (oprelvekin) is also commercially available. IL-11 from other species, including mouse, rat, pig, cow, several species of bony fish and primates, have also been cloned and sequenced.

In this specification “IL-11” refers to an IL-11 from any species and includes isoforms, fragments, variants or homologues of an IL-11 from any species. In preferred embodiments the species is human (Homo sapiens). Isoforms, fragments, variants or homologues of an IL-11 may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of immature or mature IL-11 from a given species, e.g. human. Isoforms, fragments, variants or homologues of an IL-11 may optionally be characterised by ability to bind IL-11Rα (preferably from the same species) and stimulate signal transduction in cells expressing IL-11Rα and gp130 (e.g. as described in Curtis et al. Blood, 1997, 90(11); or Karpovich et al. Mol. Hum. Reprod. 2003 9(2):75-80). A fragment of IL-11 may be of any length (by number of amino acids), although may optionally be at least 25% of the length of mature IL-11 and may have a maximum length of one of 50%, 75%, 80%, 85%, 90%, 91%, 92%, 93%, 94%, 95%, 96%, 97%, 98%, or 99% of the length of mature IL-11. A fragment of IL-11 may have a minimum length of 10 amino acids, and a maximum length of one of 15, 20, 25, 30, 40, 50, 60, 70, 80, 90, 100, 110, 120, 130, 140, 150, 160, 170, 180, 190 or 195 amino acids.

IL-11 signals through a homodimer of the ubiquitously expressed glycoprotein 130 (gp130; also known as glycoprotein 130, IL-6ST, IL-6-beta or CD 130). Gp130 is a transmembrane protein that forms one subunit of the type I cytokine receptor with the IL-6 receptor family. Specificity is gained through an individual interleukin 11 receptor subunit alpha (IL-11Rα), which does not directly participate in signal transduction, although the initial cytokine binding event to the a-receptor leads to the final complex formation with gp130.

Human gp130 (including the 22 amino acid signal peptide) is a 918 amino acid protein, and the mature form is 866 amino acids, comprising a 597 amino acid extracellular domain, a 22 amino acid transmembrane domain, and a 277 amino acid intracellular domain. The extracellular domain of the protein comprises the cytokine-binding module (CBM) of gp130. The CBM of gp130 comprises the Ig-like domain D1, and the fibronectin-type III domains D2 and D3 of gp130. The amino acid sequence of human gp 130 is available under UniProt accession no. P40189-1 (SEQ ID NO:2).

Human IL-11Rα is a 422 amino acid polypeptide (UniProt Q14626; SEQ ID NO:3) and shares ˜85% nucleotide and amino acid sequence identity with the murine IL-11Rα. Two isoforms of IL-11Rα have been reported, which differ in the cytoplasmic domain (Du and Williams, supra). The IL-11 receptor α-chain (IL-11Rα) shares many structural and functional similarities with the IL-6 receptor α-chain (IL-6Rα). The extracellular domain shows 24% amino acid identity including the characteristic conserved Trp-Ser-X-Trp-Ser (WSXWS) motif. The short cytoplasmic domain (34 amino acids) lacks the Box 1 and 2 regions that are required for activation of the JAK/STAT signalling pathway.

The receptor binding sites on murine IL-11 have been mapped and three sites-sites I, II and III-identified. Binding to gp 130 is reduced by substitutions in the site II region and by substitutions in the site III region. Site III mutants show no detectable agonist activity and have IL-11Rα antagonist activity (Cytokine Inhibitors Chapter 8; edited by Gennaro Ciliberto and Rocco Savino, Marcel Dekker, Inc. 2001).

In this specification a receptor for IL-11 (IL-11R) refers to a polypeptide or polypeptide complex capable of binding IL-11. In some embodiments an IL-11 receptor is capable of binding IL-11 and inducing signal transduction in cells expressing the receptor.

An IL-11 receptor may be from any species and includes Isoforms, fragments, variants or homologues of an IL-11 receptor from any species. In preferred embodiments the species is human (Homo sapiens).

In some embodiments the IL-11 receptor may be IL-11Rα. In some embodiments a receptor for IL-11 may be a polypeptide complex comprising IL-11Rα. In some embodiments the IL-11 receptor may be a polypeptide complex comprising IL-11Rα and gp130. In some embodiments the IL-11 receptor may be gp130 or a complex comprising gp130 to which IL-11 binds.

Isoforms, fragments, variants or homologues of an IL-11Rα may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of IL-11Rα from a given species, e.g. human. Isoforms, fragments, variants or homologues of an IL-11Rα may optionally be characterised by ability to bind IL-11 (preferably from the same species) and stimulate signal transduction in cells expressing the IL-11Rα and gp130 (e.g. as described in Curtis et al. Blood, 1997, 90(11) or Karpovich et al. Mol. Hum. Reprod. 2003 9(2):75-80). A fragment of an IL-11 receptor may be of any length (by number of amino acids), although may optionally be at least 25% of the length of the mature IL-11Rα and have a maximum length of one of 50%, 75%, 80%, 85%, 90%, 91%, 92%, 93%, 94%, 95%, 96%, 97%, 98%, or 99% of the length of the mature IL-11Rα. A fragment of an IL-11 receptor fragment may have a minimum length of 10 amino acids, and a maximum length of one of 15, 20, 25, 30, 40, 50, 100, 110, 120, 130, 140, 150, 160, 170, 180, 190, 200, 250, 300, 400, or 415 amino acids.

### IL-11 Signalling

IL-11 binds to IL-11Rα with low affinity (Kd ˜10 nmol/L), and interaction between these binding partners alone is insufficient to transduce a biological signal. The generation of a high affinity receptor (Kd ˜400 to 800 pmol/L) capable of signal transduction requires co-expression of the IL-11Rα and gp130 (Curtis et al Blood 1997; 90(11):4403-12; Hilton et al., EMBO J 13:4765, 1994; Nandurkar et al., Oncogene 12:585, 1996). Binding of IL-11 to cell-surface IL-11Rα induces heterodimerization, tyrosine phosphorylation, activation of gp130 and downstream signalling, predominantly through the mitogen-activated protein kinase (MAPK)-cascade and the Janus kinase/signal transducer and activator of transcription (Jak/STAT) pathway (Garbers and Scheller, supra).

In principle, a soluble IL-11Rα can also form biologically active soluble complexes with IL-11 (Pflanz et al., 1999 FEBS Lett, 450, 117-122) raising the possibility that, similar to IL-6, IL-11 may in some instances bind soluble IL-11Rα prior to binding cell-surface gp130 (Garbers and Scheller, supra). Curtis et al (Blood 1997 Dec. 1; 90(11):4403-12) describe expression of a soluble murine IL-11 receptor alpha chain (sIL-11R) and examined signalling in cells expressing gp130. In the presence of gp130 but not transmembrane IL-11R the sIL-11R mediated IL-11 dependent differentiation of M1 leukemic cells and proliferation in Ba/F3 cells and early intracellular events including phosphorylation of gp130, STAT3 and SHP2 similar to signalling through transmembrane IL-11R. Activation of signalling through cell-membrane bound gp130 by IL-11 bound to soluble IL-11Rα has recently been demonstrated (Lokau et al., 2016 Cell Reports 14, 1761-1773). This so-called IL-11 trans signalling may be important for disease pathogenesis, yet its role in human disease has not yet been studied. In preferred embodiments, inhibition of IL-11-mediated signalling is achieved by disrupting IL-11-mediated cis signalling.

As used herein, ‘IL-11 trans signalling’ is used to refer to signalling which is triggered by binding of IL-11 bound to IL-11Rα, to gp130. The IL-11 may be bound to IL-11Rα as a non-covalent complex. The gp130 is membrane-bound and expressed by the cell in which signalling occurs following binding of the IL-11:IL-11Rα complex to gp130. In some embodiments the IL-11Rα may be a soluble IL-11Rα. In some embodiments, the soluble IL-11Rα is a soluble (secreted) isoform of IL-11Rα (e.g. lacking a transmembrane domain). In some embodiments, the soluble IL-11Rα is the liberated product of proteolytic cleavage of the extracellular domain of cell membrane bound IL-11Rα. In some embodiments, the IL-11Rα may be cell membrane-bound, and signalling through gp130 may be triggered by binding of IL-11 bound to cell-membrane-bound IL-11Rα, termed “IL-11 cis signalling”.

IL-11-mediated signalling has been shown to stimulate hematopoiesis and thrombopoiesis, stimulate osteoclast activity, stimulate neurogenesis, inhibit adipogenesis, reduce pro inflammatory cytokine expression, modulate extracellular matrix (ECM) metabolism, and mediate normal growth control of gastrointestinal epithelial cells (Du and Williams, supra).

The physiological role of Interleukin 11 (IL-11) remains unclear. IL-11 has been most strongly linked with activation of haematopoetic cells and with platelet production. IL-11 has also been shown to confer protection against graft-vs-host-disease, inflammatory arthritis and inflammatory bowel disease, leading to IL-11 being considered an anti-inflammatory cytokine (Putoczki and Ernst, J Leukoc Biol 2010, 88(6):1109-1117). However, it is suggested that IL-11 is pro-inflammatory as well as anti-inflammatory, pro-angiogenic and important for neoplasia. Recent studies have shown that IL-11 is readily detectable during viral-induced inflammation in a mouse arthritis model and in cancers, suggesting that the expression of IL-11 can be induced by pathological stimuli. IL-11 is also linked to Stat3-dependent activation of tumour-promoting target genes in neoplastic gastrointestinal epithelium (Putoczki and Ernst, supra).

As used herein, “IL-11 signalling” and “IL-11-mediated signalling” refers to signalling mediated by binding of IL-11, or a fragment thereof having the function of the mature IL-11 molecule, to a receptor for IL-11. It will be appreciated that “IL-11 signalling” and “IL-11 mediated signalling” refer to signalling initiated by IL-11/functional fragment thereof, e.g. through binding to a receptor for IL-11. “Signalling” in turn refers to signal transduction and other cellular processes governing cellular activity.

### Kidney Injury

Aspects of the present invention relate to the diagnosis, treatment and prophylaxis of kidney injury, particularly acute kidney injury (AKI; also known as acute renal failure) and/or kidney injury, e.g. AKI.

Herein, ‘kidney injury’ refers to damage to the kidney, renal tissue, and/or one or more renal cells. Damage to a cell/tissue/organ may result from insult to the cell/tissue/organ, e.g. chemical or physical treatment/experience. In some embodiments kidney injury may be a consequence of chemical insult, e.g. in the case of drug-induced kidney injury, e.g. cisplatin-induced kidney injury. In some embodiments kidney injury may arise from physical insult, e.g. in the case of kidney injury arising as a result of crush, or kidney injury as a result of surgical damage to renal tissue, which may occur e.g. during surgery to treat a disease and/or for kidney transplantation (e.g. the kidney injury may have iatrogenic causes). In some embodiments kidney injury may be a consequence of hypoxia, e.g. as a consequence of ischaemia, or may result from reperfusion. In some embodiments kidney injury may arise from infection, immune response to infection, cancer and/or autoimmunity. Damage may be reversible or irreversible.

In some embodiments, kidney injury comprises damage to one or both of the kidneys. In some embodiments kidney injury comprises damage to one or more of a renal capsule, renal cortex, renal medulla, renal papilla, renal pyramid, renal column, renal calyx, minor calyx, major calyx, hilum, renal pelvis, ureter, renal artery and a renal vein. In some embodiments kidney injury comprises damage to one or more of a renal epithelial cell, a tubule epithelial cell, a proximal tubule epithelial cell, a distal tubule epithelial cell, a renal parietal cell, a podocyte, a loop of Henle thin segment cell, a thick ascending limb cell, a collecting duct principal cell, a collecting duct intercalated cell, and an interstitial kidney cell. In some embodiments, kidney injury comprises damage to a renal epithelial cell, e.g. a tubule epithelial cell, e.g. proximal tubule epithelial cell.

Damage to a cell/tissue/organ may be characterised by a change to the structure and/or function of the cell/tissue/organ. For example, damage to a cell/tissue/organ may be characterised by a reduction in the level of a correlate of normal function of the cell/tissue/organ, and/or an increase in a correlate of impaired function of the cell/tissue/organ. By way of illustration, damage to the kidney/renal tissue/renal cells may be characterised by a reduction in urine output by the subject experiencing kidney damage, and/or an increase in serum creatine levels in the subject experiencing kidney damage. Damage to a cell/tissue/organ may be characterised by cell death, e.g. death of cells of the damaged organ/tissue. The cell death may result from apoptosis (i.e. programmed cell death) or necrosis (premature cell death as a consequence of damage).

As used herein, ‘acute kidney injury’ generally refers to abrupt deterioration in kidney function. Acute kidney injury may be characterised by a rapid increase serum creatinine and/or a rapid reduction in urine output.

Acute kidney injury may be defined and staged in accordance with the Summary of Recommendation Statements of the Kidney Disease Improving Global Outcomes (KDIGO) group, Kidney International Supplements (2012) 2, 8-12 (hereby incorporated by reference in its entirety).

In some embodiments, acute kidney injury is defined as: (i) an increase in serum creatine by ≥0.3 mg/dl (≥26.5 μmol/l) within a 48 hour period; (ii) an increase in serum creatine to ≥1.5 times baseline which is known or presumed to have occurred within the previous 7 days; or (ill) urine volume of <0.5 ml/kg/h for 6 hours.

In some embodiments, the acute kidney injury may be stage 1, 2 or 3 acute kidney injury. Stage 1 acute kidney injury may be defined as: (i) an increase in serum creatine to 1.5-1.9 times baseline, (ii) an increase in serum creatine to ≥0.3 mg/dl (≥26.5 μmol/l), or (iii) urine volume of <0.5 ml/kg/h for 6-12 hours. Stage 2 acute kidney injury may be defined as: (i) an increase in serum creatine to 2.0-2.9 times baseline, or (ii) urine volume of <0.5 ml/kg/h for ≥12 hours. Stage 3 acute kidney injury may be defined as: (i) an increase in serum creatine to ≥3.0 times baseline, (ii) an increase in serum creatine to ≥4.0 mg/dl (≥353.6 μmol/l), (iii) initiation of renal replacement therapy, (iv) in pateints <18 years, decrease in eGFR to <35 ml/min per 1.73 m²; (v) urine volume of <0.3 ml/kg/h for ≥24 hours, or (vi) anuria for ≥12 hours.

Kidney injury may be characterised by damage to tubular epithelial cells (TECs) and/or the transition of TECs to an epithelial-to-mesenchymal cell-like phenotype (i.e. EMT). Transition of TECs to a mesenchymal cell-like phenotype may be characterised e.g. by reduced expression of E-cadherin, increased expression of SNAIL and/or increased expression of ACTA2.

The kidney injury may have any cause, examples include kidney injury resulting from mechanical (i.e. physical) damage or injury, chemical damage or injury, ischemia or genetic predisposition. The cause or damage will normally result in impaired kidney function, which may lead to kidney failure.

Mechanical damage or injury may include physical injury to the subject, to the kidney, to TECs or to podocytes. It may also include tubular obstruction/blockage, e.g. of the urinary tract.

Chemical damage or injury may be caused by a drug, medicine, toxin, herbal or dietary supplements or other chemical agent administered to, absorbed or ingested by the subject. In some embodiments the chemical damage is a side effect of the administration of such an agent to treat a disease or condition not occurring in the kidney, or occurring both in the kidney and in one or more other tissues. In some embodiments the chemical damage is a side effect of administration of a chemotherapeutic agent administered to the subject in order to prevent or treat cancer. In some embodiments the kidney injury is drug-induced kidney injury or drug-induced acute kidney injury. In some embodiments, tubular obstruction/blockage, e.g. of the urinary tract, may arise as the result of administration of certain chemical agents, e.g. sulphonamides, methotrexate, acyclovir, diethylene glycol, triamterene.

Ischemic damage may arise from a decrease in blood flow to the kidney which may be caused by a number of factors such as low blood pressure e.g. due to sepsis, blood loss or surgery, or the effect of a chemical agent, e.g. a medicine or drug, administered to the subject to treat another disease, disorder or condition. Kidney injury caused by ischemia may be ischemia-induced kidney injury, or ischemia-induced acute kidney injury. Kidney injury caused by crush injury may be ischemia-induced kidney injury with vasoconstriction or can be caused by tubular cast mechanical factors or toxic effects of circulating factors e.g. myoglobin.

In some embodiments the kidney injury, which may be AKI, is characterised by damage to, which may in some cases include or lead to death of, tubular epithelial cells (TECs) of the kidney, i.e. renal tubular epithelial cells. The TECs may be proximal or distal, both of which may be damaged in AKI, as may also the podocytes in the kidney glomerulus. Damage to TECs may also be any type of damage, injury or insult, e.g. as described above this may be mechanical, chemical or ischemic damage. Damage to TECs is a common causative factor of kidney injury, particularly AKI. Proliferation of TECs provides a mechanism for recovery and restoration of kidney function, whereas failure of TECs to proliferate can lead to disease development and progression, e.g. to chronic kidney disease and renal failure. Proliferation of podocyte precursors to restore glomerulus function may also occur, but is not as well described as TEC proliferation.

In some embodiments the kidney injury is, or is characterised by, nephrotoxicity. As used herein, nephrotoxicity refers to toxicity to the kidneys. Toxicity in turn refers to damage, e.g. as described herein. Nephrotoxicity can arise as a result of toxic effects of certain substances on renal function, and may therefore be viewed as a consequence of chemical damage or injury. As with chemical damage or injury, nephrotoxicity may be a side effect of the administration of an agent to treat a disease or condition not occurring in the kidney, or occurring both in the kidney and in one or more other tissues. In some embodiments nephrotoxicity may be a side effect of administration of a chemotherapeutic agent administered to the subject in order to prevent or treat cancer. As such, nephrotoxicity may be a form of drug-induced kidney injury or drug-induced acute kidney injury.

As described above, drug-induced kidney injury, drug-induced acute kidney injury or drug-induced nephrotoxicity may arise as a side effect of the administration of a drug or medicine intended to treat a disease, disorder or condition occurring in tissues outside the kidney, within the kidney or both. A number of drugs are known to exhibit such side-effects, which may depend on the condition being treated, the subject being treated, the dosage amount, dosage regime or mode of administration, and the extent to which the subject exhibits partial kidney failure prior to treatment. For example, the following agents have all been reported to have potential in inducing kidney injury: diuretics, B-blockers, vasodilators, ACE inhibitors, aminoglycoside antibiotics (e.g. gentamicin), amphotercicin B, cisplatin, NSAIDs (e.g. aspirin, ibuprofen, diclofenac), ciclosporin, lithium salts, cyclophosphamide, sulphonamides, methotrexate, acyclovir, diethylene glycol, triamterene, B-lactam antibiotics, vancomycin, rifampicin, ciprofloxacin, ranitidine, cimetidine, furosemide, thiazides, phenytoin. Optionally a drug-induced kidney injury is not a folate-induced kidney injury.

In some embodiments, a drug-induced kidney injury, drug-induced acute kidney injury or drug-induced nephrotoxicity may arise as a side effect of the administration of a chemotherapeutic agent intended to treat or prevent cancer in the subject. Examples of chemotherapeutic agents include alkylating agents such as cisplatin, carboplatin, mechlorethamine, cyclophosphamide, chlorambucil, ifosfamide; purine or pyrimidine anti-metabolites such as azathiopurine or mercaptopurine; alkaloids and terpenoids, such as vinca alkaloids (e.g. vincristine, vinblastine, vinorelbine, vindesine), podophyllotoxin, etoposide, teniposide, taxanes such as paclitaxel (Taxol™), docetaxel; topoisomerase inhibitors such as the type I topoisomerase inhibitors camptothecins irinotecan and topotecan, or the type II topoisomerase inhibitors amsacrine, etoposide, etoposide phosphate, teniposide; antitumor antibiotics (e.g. anthracyline antibiotics) such as dactinomycin, doxorubicin (Adriamycin™), epirubicin, bleomycin, rapamycin; antibody based agents, such as anti-VEGF, anti-TNFα, anti-IL-2, antiGpIIb/IIIa, anti-CD-52, anti-CD20, anti-RSV, anti-HER2/neu(erbB2), anti-TNF receptor, anti-EGFR antibodies, monoclonal antibodies or antibody fragments, examples include: cetuximab, panitumumab, infliximab, basiliximab, bevacizumab (Avastin®), abciximab, daclizumab, gemtuzumab, alemtuzumab, rituximab (Mabthera®), palivizumab, trastuzumab, etanercept, adalimumab, nimotuzumab; and EGFR inihibitors such as erlotinib, cetuximab, gefitinib.

In some preferred embodiments, the present invention concerns the diagnosis, treatment and/or prophylaxis of cisplatin-induced kidney injury. This may include cisplatin-induced acute kidney injury or cisplatin-induced nephrotoxicity. Cisplatin (dichlorodiamino platinum; SP-4-2)-diamminedichloroplatinum(II)) is a chemotherapeutic agent that is widely used to treat a range of cancers including head and neck, breast, lung, testis, ovarian, brain, and bladder cancers and is widely acknowledged to lead to kidney injury and dysfunction involving tubular damage and necrosis (e.g. Oh et al., Electrolyte Blood Press 2014 December; 12(2):55-65; P A Arunkumar et al., Asian Pac J Trop Biomed 2012 Aug. 2 (8):640-644). Other platinum-based chemotherapeutics agents also cause kidney damage.

Whilst it is recognised that a subject having kidney injury may also present with fibrosis of the kidney, either as a disease condition having a separable etiology or as a secondary effect of the kidney injury, in some embodiments the kidney injury being diagnosed, treated or prevented is not fibrosis of the kidney, e.g. renal fibrosis. In some embodiments the subject does not have fibrosis. In some embodiments TEC damage occurs in the absence of fibrosis. In some embodiments fibrosis arises separately (e.g. secondarily to) AKI, e.g. due to incomplete regeneration of TECs. In some embodiments, the damaged TECs in the subject are not pro-fibrotic TECs. In some embodiments, fibrosis does not arise.

### Agents Capable of Inhibiting the Action of IL-11

Aspects of the present invention involve inhibition of IL-11-mediated signalling.

Herein, ‘inhibition’ refers to a reduction, decrease or lessening relative to a control condition. For example, inhibition of the action of IL-11 by an agent capable of inhibiting IL-11-mediated signalling refers to a reduction, decrease or lessening of the extent/degree of IL-11-mediated signalling in the absence of the agent, and/or in the presence of an appropriate control agent.

Inhibition may herein also be referred to as neutralisation or antagonism. That is, an agent capable of inhibiting IL-11-mediated signalling (e.g. interaction, signalling or other activity mediated by IL-11 or an IL-11-containing complex) may be said to be a ‘neutralising’ or ‘antagonist’ agent with respect to the relevant function or process. For example, an agent which is capable of inhibiting IL-11-mediated signalling may be referred to as an agent which is capable of neutralising IL-11-mediated signalling, or may be referred to as an antagonist of IL-11-mediated signalling.

The IL-11 signalling pathway offers multiple routes for inhibition of IL-11 signalling. An agent capable of inhibiting IL-11-mediated signalling may do so e.g. through inhibiting the action of one or more factors involved in, or necessary for, signalling through a receptor for IL-11.

For example, inhibition of IL-11 signalling may be achieved by disrupting interaction between IL-11 (or an IL-11 containing complex, e.g. a complex of IL-11 and IL-11Rα) and a receptor for IL-11 (e.g. IL-11Rα, a receptor complex comprising IL-11Rα, gp130 or a receptor complex comprising IL-11Rα and gp130). In some embodiments, inhibition of IL-11-mediated signalling is achieved by inhibiting the gene or protein expression of one or more of e.g. IL-11, IL-11Rα and gp130.

In embodiments, inhibition of IL-11-mediated signalling is achieved by disrupting IL-11-mediated cis signalling but not disrupting IL-11-mediated trans signalling, e.g. inhibition of IL-11-mediated signalling is achieved by inhibiting gp130-mediated cis complexes involving membrane bound IL-11Rα. In embodiments, inhibition of IL-11-mediated signalling is achieved by disrupting IL-11-mediated trans signalling but not disrupting IL-11-mediated cis signalling, i.e. inhibition of IL-11-mediated signalling is achieved by inhibiting gp130-mediated trans signalling complexes such as IL-11 bound to soluble IL-11Rα or IL-6 bound to soluble IL-6R. In embodiments, inhibition of IL-11-mediated signalling is achieved by disrupting IL-11-mediated cis signalling and IL-11-mediated trans signalling. Any agent as described herein may be used to inhibit IL-11-mediated cis and/or trans signalling.

In other examples, inhibition of IL-11 signalling may be achieved by disrupting signalling pathways downstream of IL-11/IL-11Rα/gp130. That is, in some embodiments inhibition/antagonism of IL-11-mediated signalling comprises inhibition of a signalling pathway/process/factor downstream of signalling through the IL-11/IL-11 receptor complex.

In some embodiments inhibition/antagonism of IL-11-mediated signalling comprises inhibition of signalling through an intracellular signalling pathway which is activated by the IL-11/IL-11 receptor complex. In some embodiments inhibition/antagonism of IL-11-mediated signalling comprises inhibition of one or more factors whose expression/activity is upregulated as a consequence of signalling through the IL-11/IL-11 receptor complex.

In some embodiments, the methods of the present invention employ agents capable of inhibiting JAK/STAT signalling. In some embodiments, agents capable of inhibiting JAK/STAT signalling are capable of inhibiting the action of JAK1, JAK2, JAK3, TYK2, STAT1, STAT2, STAT3, STAT4, STAT5A, STAT5B and/or STAT6. For example, agents may be capable of inhibiting activation of JAK/STAT proteins, inhibiting interaction of JAK or STAT proteins with cell surface receptors e.g. IL-11Rα or gp130, inhibiting phosphorylation of JAK proteins, inhibiting interaction between JAK and STAT proteins, inhibiting phosphorylation of STAT proteins, inhibiting dimerization of STAT proteins, inhibiting translocation of STAT proteins to the cell nucleus, inhibiting binding of STAT proteins to DNA, and/or promoting degradation of JAK and/or STAT proteins. In some embodiments, a JAK/STAT inhibitor is selected from Ruxolitinib (Jakafi/Jakavi; Incyte), Tofacitinib (Xeljanz/Jakvinus; NIH/Pfizer), Oclacitinib (Apoquel), Baricitinib (Olumiant; Incyte/Eli Lilly), Filgotinib (G-146034/GLPG-0634; Galapagos NV), Gandotinib (LY-2784544; Eli Lilly), Lestaurtinib (CEP-701; Teva), Momelotinib (GS-0387/CYT-387; Gilead Sciences), Pacritinib (SB1518; CTI), PF-04965842 (Plizer), Upadacitinib (ABT-494; AbbVie), Peficitinib (ASP015K/JNJ-54781532; Astellas), Fedratinib (SAR302503; Celgene), Cucurbitacin I (JSI-124) and CHZ868.

In some embodiments, the methods of the present invention employ agents capable of inhibiting MAPK/ERK signalling. In some embodiments, agents capable of inhibiting MAPK/ERK signalling are capable of inhibiting the action of GRB2, inhibiting the action of RAF kinase, inhibiting the action of MEK proteins, inhibiting the activation of MAP3K/MAP2K/MAPK and/or Myc, and/or inhibiting the phosphorylation of STAT proteins. In some embodiments, agents capable of inhibiting ERK signalling are capable of inhibiting ERK p42/44. In some embodiments, an ERK inhibitor is selected from SCH772984, SC1, VX-11e, DEL-22379, Sorafenib (Nexavar; Bayer/Onyx), SB590885, PLX4720, XL281, RAF265 (Novartis), encorafenib (LGX818/Braftovi; Array BioPharma), dabralenib (Tafinlar; GSK), vemurafenib (Zelboraf; Roche), cobimetinib (Cotellic; Roche), CI-1040, PD0325901, Binimetinib (MEK162/MEKTOVI; Array BioPharma), selumetinib (AZD6244; Array/AstraZeneca) and Trametinib (GSK1120212/Mekinist; Novartis). In some embodiments, the methods of the present invention employ agents capable of inhibiting c-Jun N-terminal kinase (JNK) signalling/activity. In some embodiments, agents capable of inhibiting JNK signalling/activity are capable of inhibiting the action and/or phosphorylation of a JNK (e.g. JNK1, JNK2). In some embodiments, a JNK inhibitor is selected from SP600125, CEP 1347, TCS JNK 6o, c-JUN peptide, SU3327, AEG 3482, TCS JNK 5a, BI78D3, IQ3, SR3576, IQ1S, JIP-1 (153-163) and CC401 dihydrochloride.

Widjaja et al., bioRxiv (2019) 830018 demonstrates that NOX4 expression and activity is upregulated by signalling through IL-11/IL-11Rα/gp130. NOX4 is an NADPH oxidase, and a source of reactive oxygen species (ROS). Expression of Nox4 is upregulated in transgenic mice with hepatocyte-specific Il11 expression, and primary human hepatocytes stimulated with IL11 upregulate NOX4 expression.

In some embodiments, the present invention employs agents capable of inhibiting NOX4 expression (gene or protein expression) or function. In some embodiments, the present invention employs agents capable of inhibiting IL-11-mediated upregulation of NOX4 expression/function. Agents capable of inhibiting NOX4 expression or function may be referred to herein as NOX4 inhibitors. For example, a NOX4 inhibitor may be capable of reducing expression (e.g. gene and/or protein expression) of NOX4, reducing the level of RNA encoding NOX4, reduce the level of NOX4 protein, and/or reducing the level of a NOX4 activity (e.g. reducing NOX4-mediated NADPH oxidase activity and/or NOX4-mediated ROS production).

NOX4 inhibitors include a NOX4-binding molecules and molecules capable of reducing NOX4 expression. NOX4-binding inhibitors include peptide/nucleic acid aptamers, antibodies (and antibody fragments) and fragments of interaction partners for NOX4 which behave as antagonists of NOX4 function, and small molecules inhibitors of NOX4. Molecules capable of reducing NOX4 expression include antisense RNA (e.g. siRNA, shRNA) to NOX4. In some embodiments, a NOX4 inhibitor is selected from a NOX4 inhibitor described in Altenhofer et al., Antioxid Redox Signal. (2015) 23(5):406-427 or Augsburder et al., Redox Biol. (2019) 26:101272, such as GKT137831.

### Binding Agents

In some embodiments, agents capable of inhibiting IL-11-mediated signalling may bind to IL-11. In some embodiments, agents capable of inhibiting IL-11-mediated signalling may bind to a receptor for IL-11 (e.g. IL-11Rα, gp130, or a complex containing IL-11Rα and/or gp130). Binding of such agents may inhibit IL-11-mediated signalling by reducing/preventing the ability of IL-11 to bind to receptors for IL-11, thereby inhibiting downstream signalling. Binding of such agents may inhibit IL-11 mediated cis and/or trans-signalling by reducing/preventing the ability of IL-11 to bind to receptors for IL-11, e.g. IL-11Rα and/or gp130, thereby inhibiting downstream signalling. Agents may bind to trans-signalling complexes such as IL-11 and soluble IL-11Rα and inhibit gp130-mediated signalling.

Agents capable of binding to IL-11/an IL-11 containing complex or a receptor for IL-11 may be of any kind, but in some embodiments the agent may be an antibody, an antigen-binding fragment thereof, a polypeptide, a peptide, a nucleic acid, an oligonucleotide, an aptamer or a small molecule. The agents may be provided in isolated or purified form, or may be formulated as a pharmaceutical composition or medicament.

### Antibodies and Antigen-Binding Fragments

In some embodiments, an agent capable of binding to IL-11/an IL-11 containing complex or a receptor for IL-11 is an antibody, or an antigen-binding fragment thereof. In some embodiments, an agent capable of binding to IL-11/an IL-11 containing complex or a receptor for IL-11 is a polypeptide, e.g. a decoy receptor molecule. In some embodiments, an agent capable of binding to IL-11/an IL-11 containing complex or a receptor for IL-11 may be an aptamer.

In some embodiments, an agent capable of binding to IL-11/an IL-11 containing complex or a receptor for IL-11 is an antibody, or an antigen-binding fragment thereof. An “antibody” is used herein in the broadest sense, and encompasses monoclonal antibodies, polyclonal antibodies, monospecific and multispecific antibodies (e.g., bispecific antibodies), and antibody fragments, as long as they display binding to the relevant target molecule.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimaeric antibodies are discussed by Neuberger et al (1988, 8th International Biotechnology Symposium Part 2, 792-799). Monoclonal antibodies (mAbs) are particularly useful in the methods of the invention, and are a homogenous population of antibodies specifically targeting a single epitope on an antigen.

Polyclonal antibodies are also useful in the methods of the invention. Monospecific polyclonal antibodies are preferred. Suitable polyclonal antibodies can be prepared using methods well known in the art.

Antigen-binding fragments of antibodies, such as Fab and Fab2 fragments may also be used/provided as can genetically engineered antibodies and antibody fragments. The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

Antibodies and antigen-binding fragments according to the present disclosure comprise the complementarity-determining regions (CDRs) of an antibody which is capable of binding to the relevant target molecule (Le. IL-11/an IL-11 containing complex/a receptor for IL-11).

Antibodies capable of binding to IL-11 include e.g. monoclonal mouse anti-human IL-11 antibody clone #22626; Catalog No. MAB218 (R&D Systems, MN, USA), used e.g. in Bockhorn et al. Nat. Commun. (2013) 4(0):1393, clone 6D9A (Abbiotec), clone KT8 (Abbiotec), cione M3103F11 (BioLegend), clone 1F1 (Abnova Corporation), clone 3C6 (Abnova Corporation), clone GF1 (LifeSpan Biosciences), clone 13455 (Source BioScience), 11h3/19.6.1 (Hermann et al., Arthritis Rheum. (1998) 41(8):1388-97), AB-218-NA (R&D Systems), X203 (Ng et al., Sci Transl Med. (2019) 11(511) pii: eaaw1237, which is also published as Ng, et al., “IL-11 is a therapeutic target in idiopathic pulmonary fibrosis.” bioRxiv 336537; doi: https://doi.org/10.1101/336537) and anti-IL-11 antibodies disclosed in US 2009/0202533 A1, WO 99/59608 A2, WO 2018/109174 A2 and WO 2019/238882 A1.

In particular, anti-IL-11 antibody clone 22626 (also known as MAB218) has been shown to be an antagonist of IL-11 mediated signalling, e.g. in Schaefer et al., Nature (2017) 552(7683):110-115. Monoclonal antibody 11h3/19.6.1 is disclosed in Hermann et al., Arthritis Rheum. (1998) 41(8):1388-97 to be a neutralising anti-IL-11 IgG1. AB-218-NA from R&D Systems, used e.g. in McCoy et al., BMC Cancer (2013) 13:16, is another example of neutralizing anti-IL-11 antibody. WO 2018/109174 A2 and WO 2019/238882 A1 disclose yet further exemplary anti-IL-11 antibody antagonists of IL-11 mediated signalling. X203 (also referred to as Enx203) disclosed in Ng, et al., “IL-11 is a therapeutic target in idiopathic pulmonary fibrosis.” bioRxiv 336537; doi: https://doi.org/10.1101/336537 and WO 2019/238882 A1 is an anti-IL-11 antibody antagonist of IL-11-mediated signalling, and comprises the VH region according to SEQ ID NO:92 of WO 2019/238882 A1 (SEQ ID NO:22 of the present disclosure), and the VL region according to SEQ ID NO:94 of WO 2019/238882 A1 (SEQ ID NO:23 of the present disclosure). Humanised versions of the X203 are described in WO 2019/238882 A1, including hEnx203 which comprises the VH region according to SEQ ID NO:117 of WO 2019/238882 A1 (SEQ ID NO:30 of the present disclosure), and the VL region according to SEQ ID NO:122 of WO 2019/238882 A1 (SEQ ID NO: 31 of the present disclosure). Enx108A is a further example of an anti-IL-11 antibody antagonist of IL-11-mediated signalling, and comprises the VH region according to SEQ ID NO:8 of WO 2019/238882 A1 (SEQ ID NO:26 of the present disclosure), and the VL region according to SEQ ID NO:20 of WO 2019/238882 A1 (SEQ ID NO:27 of the present disclosure).

Antibodies capable of binding to IL-11Rα include e.g. monoclonal antibody clone 025 (Sino Biological), clone EPR5446 (Abcam), clone 473143 (R & D Systems), clones 8E2, 8D10 and 8E4 and the affinity-matured variants of 8E2 described in US 2014/0219919 A1, the monoclonal antibodies described in Blanc et al (J. Immunol Methods. 2000 Jul. 31; 241(1-2);43-59), X209 (Widjaja et al., Gastroenterology (2019) 157(3):777-792, which is also published as Widjaja, et al., “IL-11 neutralising therapies target hepatic stellate cell-induced liver inflammation and fibrosis in NASH.” bioRxiv 470062; doi: https://doi.org/10.1101/470062) antibodies disclosed in WO 2014121325 A1 and US 2013/0302277 A1, and anti-IL-11Rα antibodies disclosed in US 2009/0202533 A1, WO 99/59608 A2, WO 2018/109170 A2 and WO 2019/238884 A1.

In particular, anti-IL-11Rα antibody clone 473143 (also known as MAB1977) has been shown to be an antagonist of IL-11 mediated signalling, e.g. in Schaefer et al., Nature (2017) 552(7683):110-115. US 2014/0219919 A1 provides sequences for anti-human IL-11Rα antibody clones 8E2, 8D10 and 8E4, and discloses their ability to antagonise IL-11 mediated signalling—see e.g. to of US 2014/0219919 A1. US 2014/0219919 A1 moreover provides sequence information for an additional 62 affinity-matured variants of clone 8E2, 61 of which are disclosed to antagonise IL-11 mediated signalling-see Table 3 of US 2014/0219919 A1. WO 2018/109170 A2 and WO 2019/238884 A1 disclose yet further exemplary anti-IL-11Rα antibody antagonists of IL-11 mediated signalling. X209 (also referred to as Enx209) disclosed in Widjaja, et al., “IL-11 neutralising therapies target hepatic stellate cell-induced liver inflammation and fibrosis in NASH.” bioRxiv 470062; doi: https://doi.org/10.1101/470062 and WO 2019/238884 A1 is an anti-IL-11Rα antibody antagonist of IL-11-mediated signalling, and comprises the VH region according to SEQ ID NO:7 of WO 2019/238884 A1 (SEQ ID NO:24 of the present disclosure), and the VL region according to SEQ ID NO:14 of WO 2019/238884 A1 (SEQ ID NO:25 of the present disclosure). Humanised versions of the X209 are described in WO 2019/238884 A1, including hEnx209 which comprises the VH region according to SEQ ID NO:11 of WO 2019/238884 A1 (SEQ ID NO:32 of the present disclosure), and the VL region according to SEQ ID NO:17 of WO 2019/238884 A1 (SEQ ID NO:33 of the present disclosure).

The skilled person is well aware of techniques for producing antibodies suitable for therapeutic use in a given species/subject. For example, procedures for producing antibodies suitable for therapeutic use in humans are described in Park and Smolen Advances in Protein Chemistry (2001) 56:369-421 (hereby incorporated by reference in its entirety).

Antibodies to a given target protein (e.g. IL-11 or IL-11Rα) can be raised in model species (e.g. rodents, lagomorphs), and subsequently engineered in order to improve their suitability for therapeutic use in a given species/subject. For example, one or more amino acids of monoclonal antibodies raised by immunisation of model species can be substituted to arrive at an antibody sequence which is more similar to human germline immunoglobulin sequences (thereby reducing the potential for anti-xenogenic antibody immune responses in the human subject treated with the antibody). Modifications in the antibody variable domains may focus on the framework regions in order to preserve the antibody paratope. Antibody humanisation is a matter of routine practice in the art of antibody technology, and is reviewed e.g. in Almagro and Fransson, Frontiers in Bioscience (2008) 13:1619-1633, Safdari et al., Biotechnology and Genetic Engineering Reviews (2013) 29(2):175-186 and Lo et al., Microbiology Spectrum (2014)2(1), all of which are hereby incorporated by reference in their entirety. The requirement for humanisation can be circumvented by raising antibodies to a given target protein (e.g. IL-11 or IL-11Rα) in transgenic model species expressing human immunoglobulin genes, such that the antibodies raised in such animals are fully-human (described e.g. in Brüggemann et al., Arch Immunol Ther Exp (Warsz) (2015) 63(2):101-108, which is hereby incorporated by reference in its entirety).

Phage display techniques may also be employed to the identification of antibodies to a given target protein (e.g. IL-11 or IL-11Rα), and are well known to the skilled person. The use of phage display for the identification of fully human antibodies to human target proteins is reviewed e.g. in Hoogenboom, Nat. Biotechnol. (2005) 23, 1105-1116 and Chan et al., International Immunology (2014) 26(12):649-657, which are hereby incorporated by reference in their entirety.

The antibodies/fragments may be antagonist antibodies/fragments that inhibit or reduce a biological activity of IL-11. The antibodies/fragments may be neutralising antibodies that neutralise the biological effect of IL-11, e.g. its ability to stimulate productive signalling via an IL-11 receptor. Neutralising activity may be measured by ability to neutralise IL-11 induced proliferation in the T11 mouse plasmacytoma cell line (Nordan, R. P. et al. (1987) J. Immunol. 139:813).

IL-11- or IL-11Rα-binding antibodies can be evaluated for the ability to antagonise IL-11-mediated signalling, e.g. using the assay described in US 2014/0219919 A1 or Blanc et al (J. Immunol Methods. 2000 Jul. 31; 241(1-2);43-59. Briefly, IL-11- and IL-11Rα-binding antibodies can be evaluated in vitro for the ability to inhibit proliferation of Ba/F3 cells expressing IL-11Rα and gp130 from the appropriate species, in response to stimulation with IL-11 from the appropriate species. Alternatively, IL-11- and IL-11Rα-binding antibodies can be analysed in vitro for the ability to inhibit the fibroblast-to-myofibroblast transition following stimulation of fibroblasts with TGFβ1, by evaluation of αSMA expression (as described e.g. in WO 2018/109174 A2 (Example 6) and WO 2018/109170 A2 (Example 6), Ng et al., Sci Transl Med. (2019) 11 (511) pii: eaaw1237 and Widjaja et al., Gastroenterology (2019) 157(3):777-792).

Antibodies generally comprise six CDRs; three in the light chain variable region (VL): LC-CDR1, LC-CDR2, LC-CDR3, and three in the heavy chain variable region (VH): HC-CDR1, HC-CDR2 and HC-CDR3. The six CDRs together define the paratope of the antibody, which is the part of the antibody which binds to the target molecule. The VH region and VL region comprise framework regions (FRs) either side of each CDR, which provide a scaffold for the CDRs. From N-terminus to C-terminus, VH regions comprise the following structure: N term-[HC-FR1]-[HC-CDR1]-[HC-FR2]-[HC-CDR2]-[HC-FR3]-[HC-CDR3]-[HC-FR4]-C term; and VL regions comprise the following structure: N term-[LC-FR1]-[LC-CDR1]-[LC-FR2]-[LC-CDR2]-[LC-FR3]-[LC-CDR3]-[LC-FR4]-C term.

There are several different conventions for defining antibody CDRs and FRs, such as those described in Kabat et al., Sequences of Proteins of Immunological Interest, 5th Ed. Public Health Service, National Institutes of Health, Bethesda, MD (1991), Chothia et al., J. Mol. Biol. 196:901-917 (1987), and VBASE2, as described in Retter et al., Nucl. Acids Res. (2005) 33 (suppl 1): D671-D674. The CDRs and FRs of the VH regions and VL regions of the antibodies described herein are defined according to the Kabat system.

In some embodiments an antibody, or an antigen-binding fragment thereof, according to the present disclosure is derived from an antibody which binds specifically to IL-11 (e.g. Enx108A, Enx203 or hEnx203). In some embodiments an antibody, or an antigen-binding fragment thereof, according to the present disclosure is derived from an antibody which binds specifically to IL-11Rα (e.g. Enx209 or hEnx209).

Antibodies and antigen-binding fragments according to the present disclosure preferably inhibit IL-11-mediated signalling. Such antibodies/antigen-binding fragments may be described as being antagonists of IL-11-mediated signalling, and/or may be described as having the ability to neutralise IL-11-mediated signalling.

In some embodiments, the antibody/antigen-binding fragment comprises the CDRs of an antibody which binds to IL-11. In some embodiments the antibody/antigen-binding fragment comprises the CDRs of, or CDRs derived from, the CDRs of an IL-11-binding antibody described herein (e.g. Enx108A, Enx203 or hEnx203).

In some embodiments the antibody/antigen-binding fragment comprises a VH region incorporating the following CDRs:

(1)
HC-CDR1 having the amino acid sequence of SEQ ID NO:34
HC-CDR2 having the amino acid sequence of SEQ ID NO:35
HC-CDR3 having the amino acid sequence of SEQ ID NO:36,
or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:

(2)
LC-CDR1 having the amino acid sequence of SEQ ID NO:37
LC-CDR2 having the amino acid sequence of SEQ ID NO:38
LC-CDR3 having the amino acid sequence of SEQ ID NO:39,
or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the following CDRs:

(3)
HC-CDR1 having the amino acid sequence of SEQ ID NO:40
HC-CDR2 having the amino acid sequence of SEQ ID NO:41
HC-CDR3 having the amino acid sequence of SEQ ID NO:42,
or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:

(4)
LC-CDR1 having the amino acid sequence of SEQ ID NO:43
LC-CDR2 having the amino acid sequence of SEQ ID NO:44
LC-CDR3 having the amino acid sequence of SEQ ID NO:45,
or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the CDRs according to (1), and a VL region incorporating the CDRs according to (2). In some embodiments the antibody/antigen-binding fragment comprises a VH region incorporating the CDRs according to (3), and a VL region incorporating the CDRs according to (4).

In some embodiments the antibody/antigen-binding fragment comprises the VH region and the VL region of an antibody which binds to IL-11. In some embodiments the antibody/antigen-binding fragment comprises the VH region and VL region of, or a VH region and VL region derived from, the VH region and VL region of an IL-11-binding antibody described herein (e.g. Enx108A, Enx203 or hEnx203).

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:26 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:27.

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:22 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:23.

In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30. In some embodiments the antibody/antigen-binding fragment comprises a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31. In some embodiments the antibody/antigen-binding fragment comprises a VH region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:30 and a VL region comprising an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:31.

In some embodiments, the antibody/antigen-binding fragment comprises the CDRs of an antibody which binds to IL-11Rα. In some embodiments the antibody/antigen-binding fragment comprises the CDRs of, or CDRs derived from, the CDRs of an IL-11Rα-binding antibody described herein (e.g. Enx209 or hEnx209).

In some embodiments the antibody/antigen-binding fragment comprises a VH region incorporating the following CDRs:

(5)
HC-CDR1 having the amino acid sequence of SEQ ID NO:46
HC-CDR2 having the amino acid sequence of SEQ ID NO:47
HC-CDR3 having the amino acid sequence of SEQ ID NO:48,
or a variant thereof in which one or two or three amino acids in one or more of HC-CDR1, HC-CDR2, or HC-CDR3 are substituted with another amino acid.

In some embodiments the antibody/antigen-binding fragment comprises a VL region incorporating the following CDRs:

(6)
LC-CDR1 having the amino acid sequence of SEQ ID NO:49
LC-CDR2 having the amino acid sequence of SEQ ID NO:50
LC-CDR3 having the amino acid sequence of SEQ ID NO:51,
or a variant thereof in which one or two or three amino acids in one or more of LC-CDR1, LC-CDR2, or LC-CDR3 are substituted with another amino acid.

In some embodiments the antibody/antigen-binding fragment comprises a VH region incorporating the CDRs according to (5), and a VL region incorporating the CDRs according to (6).

In some embodiments the antibody/antigen-binding fragment comprises the VH region and the VL region of an antibody which binds to IL-11Rα. In some embodiments the antibody/antigen-binding fragment comprises the VH region and VL region of, or a VH region and VL region derived from, the VH region and VL region of an IL-11Rα-binding antibody described herein (e.g. Enx209 or hEnx209).

In embodiments in accordance with the present invention in which one or more amino acids of a reference amino acid sequence (e.g. a CDR sequence, VH region sequence or VL region sequence described herein) are substituted with another amino acid, the substitutions may conservative substitutions, for example according to the following Table. In some embodiments, amino acids in the same block in the middle column are substituted. In some embodiments, amino acids in the same line in the rightmost column are substituted:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

In some embodiments, substitution(s) may be functionally conservative. That is, in some embodiments the substitution may not affect (or may not substantially affect) one or more functional properties (e.g. target binding) of the antibody/fragment comprising the substitution relative to the equivalent unsubstituted molecule.

In some embodiments, substitution(s) relative to a reference VH region or VL region sequence may be focussed in a particular region or regions of the VH region or VL region sequence. For example, variation from a reference VH region or VL region sequence may be focussed in one or more of the framework regions (FR1, FR2, FR3 and/or FR4).

Antibodies and antigen-binding fragments according to the present disclosure may be designed and prepared using the sequences of monoclonal antibodies (mAbs) capable of binding to the relevant target molecule. Antigen-binding regions of antibodies, such as single chain variable fragment (scFv), Fab and Fab2 fragments may also be used/provided. An ‘antigen-binding region’ or ‘antigen binding fragment’ is any fragment of an antibody which is capable of binding to the target for which the given antibody is specific.

In some embodiments the antibodies/fragments comprise the VL and VH regions of an antibody which is capable of binding to IL-11, an IL-11 containing complex, or a receptor for IL-11. The VL and VH region of an antigen-binding region of an antibody together constitute the Fv region. In some embodiments the antibodies/fragments comprise or consist of the Fv region of an antibody which is capable of binding to IL-11, an IL-11 containing complex, or a receptor for IL-11. The Fv region may be expressed as a single chain wherein the VH and VL regions are covalently linked, e.g. by a flexible oligopeptide. Accordingly, antibodies/fragments may comprise or consist of an scFv comprising the VL and VH regions of an antibody which is capable of binding to IL-11, an IL-11 containing complex, or a receptor for IL-11.

The VL and light chain constant (CL) region, and the VH region and heavy chain constant 1 (CH1) region of an antigen-binding region of an antibody together constitute the Fab region. In some embodiments the antibodies/fragments comprise or consist of the Fab region of an antibody which is capable of binding to IL-11, an IL-11 containing complex, or a receptor for IL-11.

In some embodiments, antibodies/fragments comprise, or consist of, whole antibody capable of binding to IL-11, an IL-11 containing complex, or a receptor for IL-11. A “whole antibody” refers to an antibody having a structure which is substantially similar to the structure of an immunoglobulin (Ig). Different kinds of immunoglobulins and their structures are described e.g. in Schroeder and Cavacini J Allergy Clin Immunol. (2010) 125(202):S41-S52, which is hereby incorporated by reference in its entirety. Immunoglobulins of type G (i.e. IgG) are ˜150 kDa glycoproteins comprising two heavy chains and two light chains. From N- to C-terminus, the heavy chains comprise a VH followed by a heavy chain constant region comprising three constant domains (CH1, CH2, and CH3), and similarly the light chain comprises a VL followed by a CL. Depending on the heavy chain, immunoglobulins may be classed as IgG (e.g. IgG1, IgG2, IgG3, IgG4), IgA (e.g. IgA1, IgA2), IgD, IgE, or IgM. The light chain may be kappa (κ) or lambda (λ).

In some embodiments the antibody/antigen-binding fragment of the present disclosure comprises an immunoglobulin heavy chain constant sequence. In some embodiments, an immunoglobulin heavy chain constant sequence may be a human immunoglobulin heavy chain constant sequence. In some embodiments the immunoglobulin heavy chain constant sequence is, or is derived from, the heavy chain constant sequence of an IgG (e.g. IgG1, IgG2, IgG3, IgG4), IgA (e.g. IgA1, IgA2), IgD, IgE or IgM, e.g. a human IgG (e.g. hIgG1, hIgG2, hIgG3, hIgG4), higA (e.g. hIgA1, hIgA2), hIgD, hIgE or hIgM. In some the immunoglobulin heavy chain constant sequence is, or is derived from, the heavy chain constant sequence of a human IgG1 allotype (e.g. G1m1, G1m2, G1m3 or G1m17).

In some embodiments the immunoglobulin heavy chain constant sequence is, or is derived from, the constant region sequence of human immunoglobulin G 1 constant (IGHG1; UniProt: P01857-1, v1). In some embodiments the immunoglobulin heavy chain constant sequence is, or is derived from, the constant region sequence of human immunoglobulin G 1 constant (IGHG1; UniProt: P01857-1, v1) comprising substitutions K214R, D356E and L358M (i.e. the G1m3 allotype). In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:52.

In some embodiments the immunoglobulin heavy chain constant sequence is, or is derived from, the constant region sequence of human immunoglobulin G 4 constant (IGHG4; UniProt: P01861, v1). In some embodiments the immunoglobulin heavy chain constant sequence is, or is derived from, the constant region sequence of human immunoglobulin G 4 constant (IGHG4; UniProt: P01861, v1) comprising substitutions S241P and/or L248E. The S241P mutation is hinge stabilising while the L248E mutation further reduces the already low ADCC effector function of IgG4 (Davies and Sutton, Immunol Rev. 2015 November; 268(1):139-159; Angal et al Mol Immunol. 1993 January; 30(1):105-8). In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:53.

In some embodiments the antibody/antigen-binding fragment of the present disclosure comprises an immunoglobulin light chain constant sequence. In some embodiments, an immunoglobulin light chain constant sequence may be a human immunoglobulin light chain constant sequence. In some embodiments the immunoglobulin light chain constant sequence is, or is derived from, a kappa (κ) or lambda (λ) light chain, e.g. human immunoglobulin kappa constant (IGKC; Cκ; UniProt: P01834-1, v2), or human immunoglobulin lambda constant (IGLC; Cλ), e.g. IGLC1 (UniProt: P0CG04-1, v1), IGLC2 (UniProt: P0DOY2-1, v1), IGLC3 (UniProt: P0DOY3-1, v1), IGLC6 (UniProt: P0CF74-1, v1) or IGLC7 (UniProt: A0M8Q6-1, v3).

In some embodiments the antibody/antigen-binding fragment comprises an immunoglobulin light chain constant sequence. In some embodiments the immunoglobulin light chain constant sequence is, or is derived from human immunoglobulin kappa constant (IGKC; Cκ; UniProt: P01834-1, v2; SEQ ID NO:90). In some embodiments the immunoglobulin light chain constant sequence is a human immunoglobulin lambda constant (IGLC; Cλ), e.g. IGLC1, IGLC2, IGLC3, IGLC6 or IGLC7. In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:54. In some embodiments the antibody/antigen-binding fragment comprises an amino acid sequence having at least 70% sequence identity more preferably one of at least 75%, 80%, 85%, 86%, 87%, 88%, 89%, 90%, 91%, 92%, 93%, 94%, 95%, 96%, 97%, 98%, 99%, or 100%, sequence identity to the amino acid sequence of SEQ ID NO:55.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:28, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:29.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:56, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:57.

In some embodiments, the antibody/antigen-binding fragment comprises: (i) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:58, and (ii) a polypeptide comprising or consisting of an amino acid sequence having at least 70%, preferably one of 75%, 80%, 85%, 90%, 91%, 92%, 93%, 94%, 95%, 96%, 97%, 98%, 99% or 100% amino acid sequence identity to the amino acid sequence of SEQ ID NO:59.

Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)2 fragments have two antigen combining sites. In contrast, Fab, Fv, ScFv and dAb fragments are monovalent, having only one antigen combining site. Synthetic antibodies capable of binding to IL-11, an IL-11 containing complex, or a receptor for IL-11 may also be made using phage display technology as is well known in the art.

Antibodies may be produced by a process of affinity maturation in which a modified antibody is generated that has an improvement in the affinity of the antibody for antigen, compared to an unmodified parent antibody. Affinity-matured antibodies may be produced by procedures known in the art, e.g., Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):331 0-15 9 (1995); and Hawkins et al, J. Mol. Biol. 226:889-896 (1992).

Antibodies/fragments include bi-specific antibodies, e.g. composed of two different fragments of two different antibodies, such that the bi-specific antibody binds two types of antigen. The bispecific antibody comprises an antibody/fragment as described herein capable of binding to IL-11, an IL-11 containing complex, or a receptor for IL-11. The antibody may contain a different fragment having affinity for a second antigen, which may be any desired antigen. Techniques for the preparation of bi-specific antibodies are well known in the art, e.g. see Mueller, D et al., (2010 Biodrugs 24(2):89-98), Wozniak-Knopp G et al., (2010 Protein Eng Des 23(4):289-297), and Baeuerle, P A et al., (2009 Cancer Res 69(12):4941-4944). Bispecific antibodies and bispecific antigen-binding fragments may be provided in any suitable format, such as those formats described in Kontermann MAbs 2012, 4(2):182-197, which is hereby incorporated by reference in its entirety. For example, a bispecific antibody or bispecific antigen-binding fragment may be a bispecific antibody conjugate (e.g. an IgG2, F(ab′)2 or CovX-Body), a bispecific IgG or IgG-like molecule (e.g. an IgG, scFv4-Ig, IgG-scFv, scFv-IgG, DVD-Ig, IgG-sVD, sVD-IgG, 2 in 1-IgG, mAb2, or Tandemab common LC), an asymmetric bispecific IgG or IgG-like molecule (e.g. a kih IgG, kih IgG common LC, CrossMab, kih IgG-scFab, mAb-Fv, charge pair or SEED-body), a small bispecific antibody molecule (e.g. a Diabody (Db), dsDb, DART, scDb, tandAbs, tandem scFv (taFv), tandem dAb/VHH, triple body, triple head, Fab-scFv, or F(ab′)2-scFv2), a bispecific Fc and CH3 fusion protein (e.g. a taFv-Fc, Di-diabody, scDb-CH3, scFv-Fc-scFv, HCAb-VHH, scFv-kih-Fc, or scFv-kih-CH3), or a bispecific fusion protein (e.g. a scFv2-albumin, scDb-albumin, taFv-toxin, DNL-Fab3, DNL-Fab4-IgG, DNL-Fab4-IgG-cytokine2). See in particular FIG. 2 of Kontermann MAbs 2012, 4(2):182-19.

Methods for producing bispecific antibodies include chemically crosslinking antibodies or antibody fragments, e.g. with reducible disulphide or non-reducible thioether bonds, for example as described in Segal and Bast, 2001. Production of Bispecific Antibodies. Current Protocols in Immunology. 14:IV:2.13:2.13.1-2.13.16, which is hereby incorporated by reference in its entirety. For example, N-succinimidyl-3-(-2-pyridyldithio)-propionate (SPDP) can be used to chemically crosslink e.g. Fab fragments via hinge region SH-groups, to create disulfide-linked bispecific F(ab)2 heterodimers.

Other methods for producing bispecific antibodies include fusing antibody-producing hybridomas e.g. with polyethylene glycol, to produce a quadroma cell capable of secreting bispecific antibody, for example as described in D. M. and Bast, B. J. 2001. Production of Bispecific Antibodies. Current Protocols in Immunology. 14:IV:2.13:2.13.1-2.13.16.

Bispecific antibodies and bispecific antigen-binding fragments can also be produced recombinantly, by expression from e.g. a nucleic acid construct encoding polypeptides for the antigen binding molecules, for example as described in Antibody Engineering: Methods and Protocols, Second Edition (Humana Press, 2012), at Chapter 40: Production of Bispecific Antibodies: Diabodies and Tandem scFv (Hornig and Färber-Schwarz), or French, How to make bispecific antibodies, Methods Mol. Med. 2000; 40:333-339.

For example, a DNA construct encoding the light and heavy chain variable domains for the two antigen binding domains (i.e. the light and heavy chain variable domains for the antigen binding domain capable of binding to IL-11, an IL-11 containing complex, or a receptor for IL-11, and the light and heavy chain variable domains for the antigen binding domain capable of binding to another target protein), and including sequences encoding a suitable linker or dimerization domain between the antigen binding domains can be prepared by molecular cloning techniques. Recombinant bispecific antibody can thereafter be produced by expression (e.g. in vitro) of the construct in a suitable host cell (e.g. a mammalian host cell), and expressed recombinant bispecific antibody can then optionally be purified.

### Decoy Receptors

Peptide or polypeptide based agents capable of binding to IL-11 or IL-11 containing complexes may be based on the IL-11 receptor, e.g. an IL-11 binding fragment of an IL-11 receptor.

In some embodiments, the binding agent may comprise an IL-11-binding fragment of the IL-11Rα chain, and may preferably be soluble and/or exclude one or more, or all, of the transmembrane domain(s). In some embodiments, the binding agent may comprise an IL-11-binding fragment of gp130, and may preferably be soluble and/or exclude one or more, or all, of the transmembrane domain(s). Such molecules may be described as decoy receptors. Binding of such agents may inhibit IL-11 mediated cis and/or trans-signalling by reducing/preventing the ability of IL-11 to bind to receptors for IL-11, e.g. IL-11Rα or gp130, thereby inhibiting downstream signalling.

Curtis et al (Blood 1997 Dec. 1; 90(11):4403-12) report that a soluble murine IL-11 receptor alpha chain (sIL-11R) was capable of antagonizing the activity of IL-11 when tested on cells expressing the transmembrane IL-11R and gp130. They proposed that the observed IL-11 antagonism by the sIL-11R depends on limiting numbers of gp130 molecules on cells already expressing the transmembrane IL-11R.

The use of soluble decoy receptors as the basis for inhibition of signal transduction and therapeutic intervention has also been reported for other signalling molecule: receptor pairs, e.g. VEGF and the VEGF receptor (De-Chao Yu et al., Molecular Therapy (2012); 20 5, 938-947; Konner and Dupont Clin Colorectal Cancer 2004 October; 4 Suppl 2:S81-5).

As such, in some embodiments a binding agent may be a decoy receptor, e.g. a soluble receptor for IL-11 and/or IL-11 containing complexes. Competition for IL-11 and/or IL-11 containing complexes provided by a decoy receptor has been reported to lead to IL-11 antagonist action (Curtis et al., supra). Decoy IL-11 receptors are also described in WO 2017/103108 A1 and WO 2018/109168 A1, which are hereby incorporated by reference in their entirety.

Decoy IL-11 receptors preferably bind IL-11 and/or IL-11 containing complexes, and thereby make these species unavailable for binding to gp130, IL-11Rα and/or gp130:IL-11Rα receptors. As such, they act as ‘decoy’ receptors for IL-11 and IL-11 containing complexes, much in the same way that etanercept acts as a decoy receptor for TNFα. IL-11-mediated signalling is reduced as compared to the level of signalling in the absence of the decoy receptor.

Decoy IL-11 receptors preferably bind to IL-11 through one or more cytokine binding modules (CBMs). The CBMs are, or are derived from or homologous to, the CBMs of naturally occurring receptor molecules for IL-11. For example, decoy IL-11 receptors may comprise, or consist of, one or more CBMs which are from, are derived from or homologous to the CBM of gp130 and/or IL-11Rα.

In some embodiments, a decoy IL-11 receptor may comprise, or consist of, an amino acid sequence corresponding to the cytokine binding module of gp130. In some embodiments, a decoy IL-11 receptor may comprise an amino acid sequence corresponding to the cytokine binding module of IL-11Rα. Herein, an amino acid sequence which ‘corresponds’ to a reference region or sequence of a given peptide/polypeptide has at least 60%, e.g. one of at least 65%, 70%, 75%, 80%, 85%, 90%, 91%, 92%, 93%, 94%, 95%, 96%, 97%, 98% or 99% sequence identity to the amino acid sequence of the reference region/sequence.

In some embodiments a decoy receptor may be able to bind IL-11, e.g. with binding affinity of at least 100 μM or less, optionally one of 10 μM or less, 1 μM or less, 100 nM or less, or about 1 to 100 nM. In some embodiments a decoy receptor may comprise all or part of the IL-11 binding domain and may optionally lack all or part of the transmembrane domains. The decoy receptor may optionally be fused to an immunoglobulin constant region, e.g. IgG Fc region.

### Inhibitors

The present invention contemplates the use of inhibitor molecules capable of binding to one or more of IL-11, an IL-11 containing complex, IL-11Rα, gp130, or a complex containing IL-11Rα and/or gp130, and inhibiting IL-11 mediated signalling.

In some embodiments the agent is a peptide- or polypeptide-based binding agent based on IL-11, e.g. mutant, variant or binding fragment of IL-11. Suitable peptide or polypeptide based agents may bind to a receptor for IL-11 (e.g. IL-11Rα, gp130, or a complex containing IL-11Rα and/or gp130) in a manner that does not lead to initiation of signal transduction, or which produces sub-optimal signalling. IL-11 mutants of this kind may act as competitive inhibitors of endogenous IL-11.

For example, W147A is an IL-11 antagonist in which the amino acid 147 is mutated from a tryptophan to an alanine, which destroys the so-called ‘site III’ of IL-11. This mutant can bind to IL-11Rα, but engagement of the gp130 homodimer fails, resulting in efficient blockade of IL-11 signalling (Underhill-Day et al., 2003; Endocrinology 2003 August; 144(8):3406-14). Lee et al (Am J respire Cell Mol Biol. 2008 December; 39(6):739-746) also report the generation of an IL-11 antagonist mutant (a “mutein”) capable of specifically inhibiting the binding of IL-11 to IL-11Rα. IL-11 muteins are also described in WO 2009/052588 A1.

Menkhorst et al (Biology of Reproduction May 1, 2009 vol.80 no.5 920-927) describe a PEGylated IL-11 antagonist, PEGIL11A (CSL Limited, Parkvill, Victoria, Australia) which is effective to inhibit IL-11 action in female mice.

Pasqualini et al. Cancer (2015) 121(14):2411-2421 describe a ligand-directed, peptidomimetic drug, bone metastasis-targeting peptidomimetic-11 (BMTP-11) capable of binding to IL-11Rα.

In some embodiments a binding agent capable of binding to a receptor for IL-11 may be provided in the form of a small molecule inhibitor of one of IL-11Rα, gp130, or a complex containing IL-11Rα and/or gp130. In some embodiments a binding agent may be provided in the form of a small molecule inhibitor of IL-11 or an IL-11 containing complex, e.g. IL-11 inhibitor described in Lay et al., Int. J. Oncol. (2012); 41(2):759-764, which is hereby incorporated by reference in its entirety.

### Aptamers

In some embodiments, an agent capable of binding to IL-11/an IL-11 containing complex or a receptor for IL-11 (e.g. IL-11Rα, gp130, or a complex containing IL-11Rα and/or gp130) is an aptamer. Aptamers, also called nucleic acid/peptide ligands, are nucleic acid or peptide molecules characterised by the ability to bind to a target molecule with high specificity and high affinity. Almost every aptamer identified to date is a non-naturally occurring molecule.

Aptamers to a given target (e.g. IL-11, an IL-11 containing complex or a receptor for IL-11) may be identified and/or produced by the method of Systematic Evolution of Ligands by Exponential enrichment (SELEXTM), or by developing SOMAmers (slow off-rate modified aptamers) (Gold L et al. (2010) PLoS ONE 5(12):e15004). Aptamers and SELEX are described in Tuerk and Gold, Science (1990) 249(4968):505-10, and in WO 91/19813. Applying the SELEX and the SOMAmer technology includes for instance adding functional groups that mimic amino acid side chains to expand the aptamer's chemical diversity. As a result high affinity aptamers for a target may be enriched and identified.

Aptamers may be DNA or RNA molecules and may be single stranded or double stranded. The aptamer may comprise chemically modified nucleic acids, for example in which the sugar and/or phosphate and/or base is chemically modified. Such modifications may improve the stability of the aptamer or make the aptamer more resistant to degradation and may include modification at the 2′ position of ribose.

Aptamers may be synthesised by methods which are well known to the skilled person. For example, aptamers may be chemically synthesised, e.g. on a solid support. Solid phase synthesis may use phosphoramidite chemistry. Briefly, a solid supported nucleotide is detritylated, then coupled with a suitably activated nucleoside phosphoramidite to form a phosphite triester linkage. Capping may then occur, followed by oxidation of the phosphite triester with an oxidant, typically iodine. The cycle may then be repeated to assemble the aptamer (e.g., see Sinha, N. D.; Biernat, J.; McManus, J.; Köster, H. Nucleic Acids Res. 1984, 12, 4539; and Beaucage, S. L.; Lyer, R. P. (1992). Tetrahedron 48 (12):2223).

Suitable nucleic acid aptamers may optionally have a minimum length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides. Suitable nucleic acid aptamers may optionally have a maximum length of one of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides. Suitable nucleic acid aptamers may optionally have a length of one of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotides.

Aptamers may be peptides selected or engineered to bind specific target molecules. Peptide aptamers and methods for their generation and identification are reviewed in Reverdatto et al., Curr Top Med Chem. (2015) 15(12):1082-101, which is hereby incorporated by reference in its entirety. Peptide aptamers may optionally have a minimum length of one of 2, 3, 4, 5, 6, 7, 8, 9 or 10 amino acids. Peptide aptamers may optionally have a maximum length of one of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids. Suitable peptide aptamers may optionally have a length of one of 2-30, 2-25, 2-20, 5-30, 5-25 or 5-20 amino acids.

Aptamers may have K_{D}'s in the nM or pM range, e.g. less than one of 500 nM, 100 nM, 50 nM, 10 nM, 1 nM, 500 pM, 100 pM.

### Properties of IL-11 Binding Agents

Agents capable of binding to IL-11/an IL-11 containing complex or a receptor for IL-11 according to the present invention may exhibit one or more of the following properties:
Specific binding to IL-11/IL-11 containing complex or a receptor for IL-11;
Binding to IL-11/IL-11 containing complex, or a receptor for IL-11, with a KD of 10 μM or less, preferably one of ≤5 μM≤1 μM, ≤500 nM, ≤100 nM, ≤10 nM, ≤1 nM or ≤100 pM;
Inhibition of interaction between IL-11 and IL-11Rα;
Inhibition of interaction between IL-11 and gp130;
Inhibition of interaction between IL-11 and IL-11Rα:gp130 receptor complex;
Inhibition of interaction between IL-11:IL-11Rα complex and gp130.

These properties can be determined by analysis of the relevant agent in a suitable assay, which may involve comparison of the performance of the agent to suitable control agents. The skilled person is able to identify an appropriate control conditions for a given assay.

For example, a suitable negative control for the analysis of the ability of a test antibody/antigen-binding fragment to bind to IL-11/an IL-11 containing complex/a receptor for IL-11 may be an antibody/antigen-binding fragment directed against a non-target protein (i.e. an antibody/antigen-binding fragment which is not specific for IL-11/an IL-11 containing complex/a receptor for IL-11). A suitable positive control may be a known, validated (e.g. commercially available) IL-11- or IL-11 receptor-binding antibody. Controls may be of the same isotype as the putative IL-11/IL-11 containing complex/IL-11 receptor-binding antibody/antigen-binding fragment being analysed, and may e.g. have the same constant regions.

In some embodiments, the agent may be capable of binding specifically to IL-11 or an IL-11 containing complex, or a receptor for IL-11 (e.g. IL-11Rα, gp130, or a complex containing IL-11Rα and/or gp130). An agent which specifically binds to a given target molecule preferably binds the target with greater affinity. and/or with greater duration than it binds to other, non-target molecules.

In some embodiments the agent may bind to IL-11 or an IL-11 containing complex with greater affinity than the affinity of binding to one or more other members of the IL-6 cytokine family (e.g. IL-6, leukemia inhibitory factor (LIF), oncostatin M (OSM), cardiotrophin-1 (CT-1), ciliary neurotrophic factor (CNTF) and cardiotrophin-like cytokine (CLC)). In some embodiments the agent may bind to a receptor for IL-11 (e.g. IL-11Rα, gp130, or a complex containing IL-11Rα and/or gp130) with greater affinity than the affinity of binding to one or more other members of the IL-6 receptor family. In some embodiments the agent may bind with greater affinity to IL-11Rα than the affinity of binding to one or more of IL-6Rα, leukemia inhibitory factor receptor (LIFR), oncostatin M receptor (OSMR), ciliary neurotrophic factor receptor alpha (CNTFRa) and cytokine receptor-like factor 1 (CRLF1).

In some embodiments, the extent of binding of a binding agent to an non-target is less than about 10% of the binding of the agent to the target as measured, e.g., by ELISA, SPR, Bio-Layer Interferometry (BLI), MicroScale Thermophoresis (MST), or by a radioimmunoassay (RIA). Alternatively, the binding specificity may be reflected in terms of binding affinity, where the binding agent binds to IL-11, an IL-11 containing complex or a receptor for IL-11 with a K_{D} that is at least 0.1 order of magnitude (i.e. 0.1×10n, where n is an integer representing the order of magnitude) greater than the K_{D} towards another, non-target molecule. This may optionally be one of at least 0.2, 0.3, 0.4, 0.5, 0.6, 0.7, 0.8, 0.9, 1.0, 1.5, or 2.0.

Binding affinity for a given binding agent for its target is often described in terms of its dissociation constant (K_{D}). Binding affinity can be measured by methods known in the art, such as by ELISA, Surface Plasmon Resonance (SPR; see e.g. Hearty et al., Methods Mol Biol (2012) 907:411-442; or Rich et al., Anal Biochem. 2008 Feb. 1; 373(1):112-20), Bio-Layer Interferometry (see e.g. Lad et al., (2015) J Biomol Screen 20(4):498-507; or Concepcion et al., Comb Chem High Throughput Screen. 2009 September; 12(8):791-800), MicroScale Thermophoresis (MST) analysis (see e.g. Jerabek-Willemsen et al., Assay Drug Dev Technol. 2011 August; 9(4):342-353), or by a radiolabelled antigen binding assay (RIA).

In some embodiments, the agent is capable of binding to IL-11 or an IL-11 containing complex, or a receptor for IL-11 with a K_{D} of 50 μM or less, preferably one of ≤10 μM, ≤5 μM, ≤4 μM, ≤3 μM, ≤2 μM, ≤1 μM, ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM ≤3 nM, ≤2 nM, ≤1 nM, ≤500 pM, ≤400 pM, ≤300 pM, ≤200 pM, or ≤100 pM.

In some embodiments, the agent binds to IL-11, an IL-11 containing complex or a receptor for IL-11 with an affinity of binding (e.g. as determined by ELISA) of EC50=10,000 ng/ml or less, preferably one of ≤5,000 ng/ml, ≤1000 ng/ml, ≤900 ng/ml, ≤800 ng/ml, ≤700 ng/ml, ≤600 ng/ml, ≤500 ng/ml, ≤400 ng/ml, ≤300 ng/ml, ≤200 ng/ml, ≤100 ng/ml, ≤90 ng/ml, ≤80 ng/ml, ≤70 ng/ml, ≤60 ng/ml, ≤50 ng/ml, ≤40 ng/ml, ≤30 ng/ml, ≤20 ng/ml, ≤15 ng/ml, ≤10 ng/ml, ≤7.5 ng/ml, ≤5 ng/ml, ≤2.5 ng/ml, or ≤1 ng/ml. Such ELISAs can be performed e.g. as described in Antibody Engineering, vol. 1 (2nd Edn), Springer Protocols, Springer (2010), Part V, pp657-665.

In some embodiments, the agent binds to IL-11 or an IL-11-containing complex in a region which is important for binding to a receptor for the IL-11 or IL-11-containing complex, e.g. gp130 or IL-11Rα, and thereby inhibits interaction between IL-11 or an IL-11-containing complex and a receptor for IL-11, and/or signalling through the receptor. In some embodiments, the agent binds to a receptor for IL-11 in a region which is important for binding to IL-11 or an IL-11-containing complex, and thereby inhibits interaction between IL-11 or an IL-11-containing complex and a receptor for IL-11, and/or signalling through the receptor.

The ability of a given binding agent (e.g. an agent capable of binding IL-11/an IL-11 containing complex or a receptor for IL-11) to inhibit interaction between two proteins can be determined for example by analysis of interaction in the presence of, or following incubation of one or both of the interaction partners with, the binding agent. An example of a suitable assay to determine whether a given binding agent is capable of inhibiting interaction between two interaction partners is a competition ELISA.

A binding agent which is capable of inhibiting a given interaction (e.g. between IL-11 and IL-11Rα, or between IL-11 and gp130, or between IL-11 and IL-11Rα:gp130, or between IL-11:IL-11Rα and gp130) is identified by the observation of a reduction/decrease in the level of interaction between the interaction partners in the presence of—or following incubation of one or both of the interaction partners with—the binding agent, as compared to the level of interaction in the absence of the binding agent (or in the presence of an appropriate control binding agent). Suitable analysis can be performed in vitro, e.g. using recombinant interaction partners or using cells expressing the interaction partners. Cells expressing interaction partners may do so endogenously, or may do so from nucleic acid introduced into the cell. For the purposes of such assays, one or both of the interaction partners and/or the binding agent may be labelled or used in conjunction with a detectable entity for the purposes of detecting and/or measuring the level of interaction. For example, the agent may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding agent may be directly labelled with a detectable label or it may be indirectly labelled. For example, the binding agent may be unlabelled, and detected by another binding agent which is itself labelled.

Alternatively, the second binding agent may have bound to it biotin and binding of labelled streptavidin to the biotin may be used to indirectly label the first binding agent.

Ability of a binding agent to inhibit interaction between two binding partners can also be determined by analysis of the downstream functional consequences of such interaction, e.g. IL-11-mediated signalling. For example, downstream functional consequences of interaction between IL-11 and IL-11Rα:gp130 or between IL-11:IL-11Rα and gp130 may include e.g. a process mediated by IL-11, or gene/protein expression of e.g. collagen or IL-11.

Inhibition of interaction between IL-11 or an IL-11 containing complex and a receptor for IL-11 can be analysed using 3H-thymidine incorporation and/or Ba/F3 cell proliferation assays such as those described in e.g. Curtis et al. Blood, 1997, 90 (11) and Karpovich et al. Mol. Hum. Reprod. 2003 9(2):75-80. Ba/F3 cells co-express IL-11Rα and gp130.

In some embodiments, the binding agent may be capable of inhibiting interaction between IL-11 and IL-11Rα to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and IL-11Rα in the absence of the binding agent (or in the presence of an appropriate control binding agent). In some embodiments, the binding agent may be capable of inhibiting interaction between IL-11 and IL-11Rα to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα in the absence of the binding agent (or in the presence of an appropriate control binding agent).

In some embodiments, the binding agent may be capable of inhibiting interaction between IL-11 and gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and gp130 in the absence of the binding agent (or in the presence of an appropriate control binding agent). In some embodiments, the binding agent may be capable of inhibiting interaction between IL-11 and gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and gp130 in the absence of the binding agent (or in the presence of an appropriate control binding agent).

In some embodiments, the binding agent may be capable of inhibiting interaction between IL-11 and IL-11Rα:gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11 and IL-11Rα:gp130 in the absence of the binding agent (or in the presence of an appropriate control binding agent). In some embodiments, the binding agent may be capable of inhibiting interaction between IL-11 and IL-11Rα:gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11 and IL-11Rα:gp130 in the absence of the binding agent (or in the presence of an appropriate control binding agent).

In some embodiments, the binding agent may be capable of inhibiting interaction between IL-11:IL-11Rαcomplex and gp130 to less than 100%, e.g. one of 99% or less, 95% or less, 90% or less, 85% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of interaction between IL-11:IL-11Rα complex and gp130 in the absence of the binding agent (or in the presence of an appropriate control binding agent). In some embodiments, the binding agent is capable of inhibiting interaction between IL-11:IL-11Rα complex and gp130 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of interaction between IL-11:IL-11Rα complex and gp130 in the absence of the binding agent.

### Agents Capable of Reducing Expression of IL-11 or an IL-11 Receptor

In aspects of the present invention the agent capable of inhibiting IL-11-mediated signalling may be capable of preventing or reducing the expression of one or more of IL-11, IL-11Rα or gp130.

Expression may be gene or protein expression, and may be determined as described herein or by methods in the art that will be well known to a skilled person. Expression may be by a cell/tissue/organ/organ system of a subject.

Suitable agents may be of any kind, but in some embodiments an agent capable of preventing or reducing the expression of one or more of IL-11, IL-11Rα or gp130 may be a small molecule or an oligonucleotide.

An agent capable of preventing or reducing of the expression of one or more of IL-11, IL-11Rα or gp130 may do so e.g. through inhibiting transcription of the gene encoding IL-11, IL-11Rα or gp130, inhibiting post-transcriptional processing of RNA encoding IL-11, IL-11Rα or gp130, reducing the stability of RNA encoding IL-11, IL-11Rα or gp130, promoting degradation of RNA encoding IL-11, IL-11Rα or gp130, inhibiting post-translational processing of IL-11, IL-11Rα or gp130 polypeptide, reducing the stability of IL-11, IL-11Rα or gp130 polypeptide or promoting degradation of IL-11, IL-11Rα or gp130 polypeptide.

Taki et al. Clin Exp Immunol (1998) April; 112(1):133-138 reported a reduction in the expression of IL-11 in rheumatoid synovial cells upon treatment with indomethacin, dexamethasone or interferon-gamma (IFNγ).

The present invention contemplates the use of antisense nucleic acid to prevent/reduce expression of IL-11, IL-11Rα or gp130. In some embodiments, an agent capable of preventing or reducing the expression of IL-11, IL-11Rα or gp130 may cause reduced expression by RNA interference (RNAi).

In some embodiments, the agent may be an inhibitory nucleic acid, such as antisense or small interfering RNA, including but not limited to shRNA or siRNA.

In some embodiments the inhibitory nucleic acid is provided in a vector. For example, in some embodiments the agent may be a lentiviral vector encoding shRNA for one or more of IL-11, IL-11Rα or gp130.

Oligonucleotide molecules, particularly RNA, may be employed to regulate gene expression. These include antisense oligonucleotides, targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

An antisense oligonucleotide is an oligonucleotide, preferably single-stranded, that targets and binds, by complementary sequence binding, to a target oligonucleotide, e.g. mRNA. Where the target oligonucleotide is an mRNA, binding of the antisense to the mRNA blocks translation of the mRNA and expression of the gene product. Antisense oligonucleotides may be designed to bind sense genomic nucleic acid and inhibit transcription of a target nucleotide sequence.

In view of the known nucleic acid sequences for IL-11, IL-11Rα and gp130 (e.g. the known mRNA sequences available from GenBank under Accession No.s: BC012506.1 GI:15341754 (human IL-11), BC134354.1 GI:126632002 (mouse IL-11), AF347935.1 GI:13549072 (rat IL-11), NM_001142784.2 GI:391353394 (human IL-11Rα), NM_001163401.1 GI:254281268 (mouse IL-11Rα), NM_139116.1 GI:20806172 (rat IL-11Rα), NM_001190981.1 GI:300244534 (human gp130), NM_010560.3 GI:225007624 (mouse gp130), NM_001008725.3 GI:300244570 (rat gp130) oligonucleotides may be designed to repress or silence the expression of IL-11, IL-11Rα or gp130.

Such oligonucleotides may have any length, but may preferably be short, e.g. less than 100 nucleotides, e.g. 10-40 nucleotides, or 20-50 nucleotides, and may comprise a nucleotide sequence having complete- or near-complementarity (e.g. 80%, 85%, 90%, 91%, 92%, 93%, 94%, 95%, 96%, 97%, 98%, 99% or 100% complementarity) to a sequence of nucleotides of corresponding length in the target oligonucleotide, e.g. the IL-11, IL-11Rα or gp130 mRNA. The complementary region of the nucleotide sequence may have any length, but is preferably at least 5, and optionally no more than 50, nucleotides long, e.g. one of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides.

Repression of expression of IL-11, IL-11Rα or gp130 will preferably result in a decrease in the quantity of IL-11, IL-11Rα or gp130 expressed by a cell/tissue/organ/organ system/subject. For example, in a given cell the repression of IL-11, IL-11Rα or gp130 by administration of a suitable nucleic acid will result in a decrease in the quantity of IL-11, IL-11Rα or gp130 expressed by that cell relative to an untreated cell. Repression may be partial. Preferred degrees of repression are at least 50%, more preferably one of at least 60%, 70%, 80%, 85% or 90%. A level of repression between 90% and 100% is considered a ‘silencing’ of expression or function.

A role for the RNAi machinery and small RNAs in targeting of heterochromatin complexes and epigenetic gene silencing at specific chromosomal loci has been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA. RNAi based therapeutics have been progressed into Phase I, II and III clinical trials for a number of indications (Nature 2009 Jan. 22; 457(7228):426-433).

In the art, these RNA sequences are termed “short or small interfering RNAs” (siRNAs) or “microRNAs” (miRNAs) depending on their origin. Both types of sequence may be used to down-regulate gene expression by binding to complementary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in John et al, PLoS Biology, 11(2), 1862-1879, 2004.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design suitable siRNA and miRNA sequences, for example using resources such the Ambion siRNA finder. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example Myers (2003) Nature Biotechnology 21:324-328). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (Shinagawa et al., Genes and Dev., 17, 1340-5, 2003).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of IL-11, IL-11Rα or gp130. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of IL-11, IL-11Rα or gp130.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific (e.g. heart, liver, or kidney specific) promoter. In a further embodiment, the siRNA, longer dsRNA or miRNA is produced exogenously (in vitro) by transcription from a vector.

Suitable vectors may be oligonucleotide vectors configured to express the oligonucleotide agent capable of IL-11, IL-11Rα or gp130 repression. Such vectors may be viral vectors or plasmid vectors. The therapeutic oligonucleotide may be incorporated in the genome of a viral vector and be operably linked to a regulatory sequence, e.g. promoter, which drives its expression. The term “operably linked” may include the situation where a selected nucleotide sequence and regulatory nucleotide sequence are covalently linked in such a way as to place the expression of a nucleotide sequence under the influence or control of the regulatory sequence. Thus a regulatory sequence is operably linked to a selected nucleotide sequence if the regulatory sequence is capable of effecting transcription of a nucleotide sequence which forms part or all of the selected nucleotide sequence.

Viral vectors encoding promoter-expressed siRNA sequences are known in the art and have the benefit of long term expression of the therapeutic oligonucleotide. Examples include lentiviral (Nature 2009 Jan. 22; 457(7228):426-433), adenovirus (Shen et al., FEBS Lett 2003 Mar. 27; 539(1-3)111-4) and retroviruses (Barton and Medzhitov PNAS Nov. 12, 2002 vol.99, no.23 14943-14945).

In other embodiments a vector may be configured to assist delivery of the therapeutic oligonucleotide to the site at which repression of IL-11, IL-11Rα or gp130 expression is required. Such vectors typically involve complexing the oligonucleotide with a positively charged vector (e.g., cationic cell penetrating peptides, cationic polymers and dendrimers, and cationic lipids); conjugating the oligonucleotide with small molecules (e.g., cholesterol, bile acids, and lipids), polymers, antibodies, and RNAs; or encapsulating the oligonucleotide in nanoparticulate formulations (Wang et al., AAPS J. 2010 December; 12(4):492-503).

In one embodiment, a vector may comprise a nucleic acid sequence in both the sense and antisense orientation, such that when expressed as RNA the sense and antisense sections will associate to form a double stranded RNA.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′position and other than a phosphate group at the 5′position. Thus modified nucleotides may also include 2′substituted sugars such as 2′-O-methyl-; 2′-O-alkyl; 2′-O-allyl; 2′-S-alkyl; 2′-S-allyl; 2′-fluoro-; 2′-halo or azido-ribose, carbocyclic sugar analogues, a-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine,5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5methoxyuracil, 2 methylthio-N6-isopentenyladenine, uracil-5-oxyacetic acid methyl ester, pseudouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15, 485-490 (2001); Hammond, S. M., et al., Nature Rev. Genet. 2, 110-1119 (2001); Tuschl, T. Chem. Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001 Nature 411:494-498).

Accordingly, the invention provides nucleic acid that is capable, when suitably introduced into or expressed within a mammalian, e.g. human, cell that otherwise expresses IL-11, IL-11Rα or gp130, of suppressing IL-11, IL-11Rα or gp130 expression by RNAi.

Nucleic acid sequences for IL-11, IL-11Rα and gp130 (e.g. the known mRNA sequences available from GenBank under Accession No.s: BC012506.1 GI:15341754 (human IL-11), BC134354.1 GI:126632002 (mouse IL-11), AF347935.1 GI:13549072 (rat IL-11), NM_001142784.2 GI:391353394 (human IL-11Rα), NM_001163401.1 GI:254281268 (mouse IL-11Rα), NM_139116.1 GI:20806172 (rat IL-11Rα), NM_001190981.1 GI:300244534 (human gp130), NM_010560.3 GI:225007624 (mouse gp130), NM_001008725.3 GI:300244570 (rat gp130)) oligonucleotides may be designed to repress or silence the expression of IL-11, IL-11Rα or gp130.

The nucleic acid may have substantial sequence identity to a portion of IL-11, IL-11Rα or gp130 mRNA, e.g. as defined in GenBank accession no. NM_000641.3 GI:391353405 (IL-11), NM_001142784.2 GI:391353394 (IL-11Rα), NM_001190981.1 GI:300244534 (gp130) or the complementary sequence to said mRNA.

The nucleic acid may be a double-stranded siRNA. (As the skilled person will appreciate, and as explained further below, a siRNA molecule may include a short 3′ DNA sequence also.)

Alternatively, the nucleic acid may be a DNA (usually double-stranded DNA) which, when transcribed in a mammalian cell, yields an RNA having two complementary portions joined via a spacer, such that the RNA takes the form of a hairpin when the complementary portions hybridise with each other. In a mammalian cell, the hairpin structure may be cleaved from the molecule by the enzyme DICER, to yield two distinct, but hybridised, RNA molecules.

In some preferred embodiments, the nucleic acid is generally targeted to the sequence of one of SEQ ID NOs 4 to 7 (IL-11) or to one of SEQ ID NOs 8 to 11 (IL-11Rα).

Only single-stranded (i.e. non self-hybridised) regions of an mRNA transcript are expected to be suitable targets for RNAi. It is therefore proposed that other sequences very close in the IL-11 or IL-11Rα mRNA transcript to the sequence represented by one of SEQ ID NOs 4 to 7 or 8 to 11 may also be suitable targets for RNAL. Such target sequences are preferably 17-23 nucleotides in length and preferably overlap one of SEQ ID NOs 4 to 7 or 8 to 11 by at least 1, 2, 3, 4, 5, 6, 7, 8, 9, 10, 11, 12, 13, 14, 15, 16, 17, 18 or all 19 nucleotides (at either end of one of SEQ ID NOs 4 to 7 or 8 to 11).

Accordingly, the invention provides nucleic acid that is capable, when suitably introduced into or expressed within a mammalian cell that otherwise expresses IL-11 or IL-11Rα, of suppressing IL-11 or IL-11Rα expression by RNAi, wherein the nucleic acid is generally targeted to the sequence of one of SEQ ID NOs 4 to 7 or 8 to 11.

By “generally targeted” the nucleic acid may target a sequence that overlaps with SEQ ID NOs 4 to 7 or 8 to 11. In particular, the nucleic acid may target a sequence in the mRNA of human IL-11 or IL-11Rα that is slightly longer or shorter than one of SEQ ID NOs 4 to 7 or 8 to 11 (preferably from 17-23 nucleotides in length), but is otherwise identical to one of SEQ ID NOs 4 to 7 or 8 to 11.

It is expected that perfect identity/complementarity between the nucleic acid of the invention and the target sequence, although preferred, is not essential. Accordingly, the nucleic acid of the invention may include a single mismatch compared to the mRNA of IL-11 or IL-11Rα. It is expected, however, that the presence of even a single mismatch is likely to lead to reduced efficiency, so the absence of mismatches is preferred. When present, 3′ overhangs may be excluded from the consideration of the number of mismatches.

The term “complementarity” is not limited to conventional base pairing between nucleic acid consisting of naturally occurring ribo- and/or deoxyribonucleotides, but also includes base pairing between mRNA and nucleic acids of the invention that include non-natural nucleotides.

In one embodiment, the nucleic acid (herein referred to as double-stranded siRNA) includes the double-stranded RNA sequences shown in SEQ ID NOs 12 to 15. In another embodiment, the nucleic acid (herein referred to as double-stranded siRNA) includes the double-stranded RNA sequences shown in SEQ ID NOs 16 to 19.

However, it is also expected that slightly shorter or longer sequences directed to the same region of IL-11 or IL-11Rα mRNA will also be effective. In particular, it is expected that double-stranded sequences between 17 and 23 bp in length will also be effective.

The strands that form the double-stranded RNA may have short 3′ dinucleotide overhangs, which may be DNA or RNA. The use of a 3′ DNA overhang has no effect on siRNA activity compared to a 3′ RNA overhang, but reduces the cost of chemical synthesis of the nucleic acid strands (Elbashir et al., 2001c). For this reason, DNA dinucleotides may be preferred.

When present, the dinucleotide overhangs may be symmetrical to each other, though this is not essential. Indeed, the 3′ overhang of the sense (upper) strand is irrelevant for RNAi activity, as it does not participate in mRNA recognition and degradation (Elbashir et al., 2001a, 2001b, 2001c).

While RNAi experiments in Drosophila show that antisense 3′ overhangs may participate in mRNA recognition and targeting (Elbashir et al. 2001c), 3′ overhangs do not appear to be necessary for RNAi activity of siRNA in mammalian cells. Incorrect annealing of 3′ overhangs is therefore thought to have little effect in mammalian cells (Elbashir et al. 2001c; Czauderna et al. 2003).

Any dinucleotide overhang may therefore be used in the antisense strand of the siRNA. Nevertheless, the dinucleotide is preferably -UU or -UG (or -TT or -TG if the overhang is DNA), more preferably -UU (or -TT). The -UU (or -TT) dinucleotide overhang is most effective and is consistent with (i.e. capable of forming part of) the RNA polymerase III end of transcription signal (the terminator signal is TTTTT). Accordingly, this dinucleotide is most preferred. The dinucleotides AA, CC and GG may also be used, but are less effective and consequently less preferred.

Moreover, the 3′ overhangs may be omitted entirely from the siRNA.

The invention also provides single-stranded nucleic acids (herein referred to as single-stranded siRNAs) respectively consisting of a component strand of one of the aforementioned double-stranded nucleic acids, preferably with the 3′-overhangs, but optionally without. The invention also provides kits containing pairs of such single-stranded nucleic acids, which are capable of hybridising with each other in vitro to form the aforementioned double-stranded siRNAs, which may then be introduced into cells.

The invention also provides DNA that, when transcribed in a mammalian cell, yields an RNA (herein also referred to as an shRNA) having two complementary portions which are capable of self-hybridising to produce a double-stranded motif, e.g. including a sequence selected from the group consisting of SEQ ID NOs:12 to 15 or 16 to 19 or a sequence that differs from any one of the aforementioned sequences by a single base pair substitution.

The complementary portions will generally be joined by a spacer, which has suitable length and sequence to allow the two complementary portions to hybridise with each other. The two complementary (i.e. sense and antisense) portions may be joined 5′-3′ in either order. The spacer will typically be a short sequence, of approximately 4-12 nucleotides, preferably 4-9 nucleotides, more preferably 6-9 nucleotides.

Preferably the 5′ end of the spacer (immediately 3′ of the upstream complementary portion) consists of the nucleotides -UU- or -UG-, again preferably -UU- (though, again, the use of these particular dinucleotides is not essential). A suitable spacer, recommended for use in the pSuper system of OligoEngine (Seattle, Washington, USA) is UUCAAGAGA. In this and other cases, the ends of the spacer may hybridise with each other, e.g. elongating the double-stranded motif beyond the exact sequences of SEQ ID NOs 12 to 15 or 16 to 19 by a small number (e.g. 1 or 2) of base pairs.

Similarly, the transcribed RNA preferably includes a 3′ overhang from the downstream complementary portion. Again, this is preferably-UU or-UG, more preferably-UU.

Such shRNA molecules may then be cleaved in the mammalian cell by the enzyme DICER to yield a double-stranded siRNA as described above, in which one or each strand of the hybridised dsRNA includes a 3′ overhang.

Techniques for the synthesis of the nucleic acids of the invention are of course well known in the art.

The skilled person is well able to construct suitable transcription vectors for the DNA of the invention using well-known techniques and commercially available materials. In particular, the DNA will be associated with control sequences, including a promoter and a transcription termination sequence.

Of particular suitability are the commercially available pSuper and pSuperior systems of OligoEngine (Seattle, Washington, USA). These use a polymerase-III promoter (H1) and a T5 transcription terminator sequence that contributes two U residues at the 3′ end of the transcript (which, after DICER processing, provide a 3′ UU overhang of one strand of the siRNA).

Another suitable system is described in Shin et al. (RNA, 2009 May; 15(5):898-910), which uses another polymerase-III promoter (U6).

The double-stranded siRNAs of the invention may be introduced into mammalian cells in vitro or in vivo using known techniques, as described below, to suppress expression of IL-11 or a receptor for IL-11.

Similarly, transcription vectors containing the DNAs of the invention may be introduced into tumour cells in vitro or in vivo using known techniques, as described below, for transient or stable expression of RNA, again to suppress expression of IL-11 or a receptor for IL-11.

Accordingly, the invention also provides a method of suppressing expression of IL-11 or a receptor for IL-11 in a mammalian, e.g. human, cell, the method comprising administering to the cell a double-stranded siRNA of the invention or a transcription vector of the invention.

Similarly, the invention further provides a method of treating kidney injury and/or a disorder, disease or condition associated with kidney injury, the method comprising administering to a subject a double-stranded siRNA of the invention or a transcription vector of the invention.

The invention further provides the double-stranded siRNAs of the invention and the transcription vectors of the invention, for use in a method of treatment, preferably a method of treating kidney injury and/or a disorder, disease or condition associated with kidney injury.

The invention further provides the use of the double-stranded siRNAs of the invention and the transcription vectors of the invention in the preparation of a medicament for the treatment of kidney injury and/or a disorder, disease or condition associated with kidney injury.

The invention further provides a composition comprising a double-stranded siRNA of the invention or a transcription vector of the invention in admixture with one or more pharmaceutically acceptable carriers. Suitable carriers include lipophilic carriers or vesicles, which may assist in penetration of the cell membrane.

Materials and methods suitable for the administration of siRNA duplexes and DNA vectors of the invention are well known in the art and improved methods are under development, given the potential of RNAi technology.

Generally, many techniques are available for introducing nucleic acids into mammalian cells. The choice of technique will depend on whether the nucleic acid is transferred into cultured cells in vitro or in vivo in the cells of a patient. Techniques suitable for the transfer of nucleic acid into mammalian cells in vitro include the use of liposomes, electroporation, microinjection, cell fusion, DEAE, dextran and calcium phosphate precipitation. In vivo gene transfer techniques include transfection with viral (typically retroviral) vectors and viral coat protein-liposome mediated transfection (Dzau et al. (2003) Trends in Biotechnology 11, 205-210).

In particular, suitable techniques for cellular administration of the nucleic acids of the invention both in vitro and in vivo are disclosed in the following articles:

General reviews: Borkhardt, A. 2002. Blocking oncogenes in malignant cells by RNA interference—new hope for a highly specific cancer treatment? Cancer Cell. 2:167-8. Hannon, G. J. 2002. RNA interference. Nature. 418:244-51. McManus, M. T., and P. A. Sharp. 2002. Gene silencing in mammals by small interfering RNAs. Nat Rev Genet. 3:737-47. Scherr, M., M. A. Morgan, and M. Eder. 2003b. Gene silencing mediated by small interfering RNAs in mammalian cells. Curr Med Chem. 10:245-56. Shuey, D. J., D. E. McCallus, and T. Giordano. 2002. RNAi: gene-silencing in therapeutic intervention. Drug Discov Today. 7:1040-6.

Systemic delivery using liposomes: Lewis, D. L., J. E. Hagstrom, A. G. Loomis, J. A. Wollf, and H. Herweijer. 2002. Efficient delivery of siRNA for inhibition of gene expression in postnatal mice. Nat Genet. 32:107-8. Paul, C. P., P. D. Good, I. Winer, and D. R. Engelke. 2002. Effective expression of small interfering RNA in human cells. Nat Biotechnol. 20:505-8. Song, E., S. K. Lee, J. Wang, N. Ince, N. Ouyang, J. Min, J. Chen, P. Shankar, and J. Lieberman. 2003. RNA interference targeting Fas protects mice from fulminant hepatitis. Nat Med. 9:347-51. Sorensen, D. R., M. Leirdal, and M. Sioud. 2003. Gene silencing by systemic delivery of synthetic siRNAs in adult mice. J Mol Biol. 327:761-6.

Virus mediated transfer: Abbas-Terki, T., W. Blanco-Bose, N. Deglon, W. Pralong, and P. Aebischer. 2002. Lentiviral-mediated RNA interference. Hum Gene Ther. 13:2197-201. Barton, G. M., and R. Medzhitov. 2002. Retroviral delivery of small interfering RNA into primary cells. Proc Natl Acad Sci U S A. 99:14943-5. Devroe, E., and P. A. Silver. 2002. Retrovirus-delivered siRNA. BMC Biotechnol. 2:15. Lori, F., P. Guallini, L. Galluzzi, and J. Lisziewicz. 2002. Gene therapy approaches to HIV infection. Am J Pharmacogenomics. 2:245-52. Matta, H., B. Hozayev, R. Tomar, P. Chugh, and P. M. Chaudhary. 2003. Use of lentiviral vectors for delivery of small interfering RNA. Cancer Biol Ther. 2:206-10. Qin, X. F., D. S. An, I. S. Chen, and D. Baltimore. 2003. Inhibiting HIV-1 infection in human T cells by lentiviral-mediated delivery of small interfering RNA against CCR5. Proc Natl Acad Sci U S A. 100:183-8. Scherr, M., K. Battmer, A. Ganser, and M. Eder. 2003a. Modulation of gene expression by lentiviral-mediated delivery of small interfering RNA. Cell Cycle. 2:251-7. Shen, C., A. K. Buck, X. Liu, M. Winkler, and S. N. Reske. 2003. Gene silencing by adenovirus-delivered siRNA. FEBS Lett. 539:111-4.

Peptide delivery: Morris, M. C., L. Chaloin, F. Heitz, and G. Divita. 2000. Translocating peptides and proteins and their use for gene delivery. Curr Opin Biotechnol. 11:461-6. Simeoni, F., M. C. Morris, F. Heitz, and G. Divita. 2003. Insight into the mechanism of the peptide-based gene delivery system MPG: implications for delivery of siRNA into mammalian cells. Nucleic Acids Res. 31:2717-24. Other technologies that may be suitable for delivery of siRNA to the target cells are based on nanoparticles or nanocapsules such as those described in U.S. Pat. Nos. 6,649,192B and 5,843,509B.

### Inhibition of IL-11-Mediated Signalling

In embodiments of the present invention, agents capable of inhibiting the action of IL-11 may possess one or more of the following functional properties:
Inhibition of signalling mediated by IL-11;
Inhibition of signalling mediated by binding of IL-11 to IL-11Rα:gp130 receptor complex;
Inhibition of signalling mediated by binding of IL-11:IL-11Rα complex to gp130 (i.e. IL-11 trans signalling);
Inhibition of a process mediated by IL-11;
Inhibition of gene/protein expression of IL-11 and/or IL-11Rα.

These properties can be determined by analysis of the relevant agent in a suitable assay, which may involve comparison of the performance of the agent to suitable control agents. The skilled person is able to identify an appropriate control conditions for a given assay.

IL-11-mediated signalling and/or processes mediated by IL-11 includes signalling mediated by fragments of IL-11 and polypeptide complexes comprising IL-11 or fragments thereof. IL-11-mediated signalling may be signalling mediated by human IL-11 and/or mouse IL-11. Signalling mediated by IL-11 may occur following binding of IL-11 or an IL-11 containing complex to a receptor to which IL-11 or said complex binds.

In some embodiments, an agent may be capable of inhibiting the biological activity of IL-11 or an IL-11-containing complex.

In some embodiments, the agent is an antagonist of one or more signalling pathways which are activated by signal transduction through receptors comprising IL-11Rα and/or gp130, e.g. IL-11Rα:gp130. In some embodiments, the agent is capable of inhibiting signalling through one or more immune receptor complexes comprising IL-11Rα and/or gp130, e.g. IL-11Rα:gp130. In various aspects of the present invention, an agent provided herein is capable of inhibiting IL-11-mediated cis and/or trans signalling. In some embodiments in accordance with the various aspects of the present invention an agent provided herein is capable of inhibiting IL-11-mediated cis signalling.

In some embodiments, the agent may be capable of inhibiting IL-11-mediated signalling to less than 100%, e.g. one of 99% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of signalling in the absence of the agent (or in the presence of an appropriate control agent). In some embodiments, the agent is capable of reducing IL-11-mediated signalling to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of signalling in the absence of the agent (or in the presence of an appropriate control agent).

In some embodiments, the IL-11-mediated signalling may be signalling mediated by binding of IL-11 to IL-11Rα:gp130 receptor. Such signalling can be analysed e.g. by treating cells expressing IL-11Rα and gp130 with IL-11, or by stimulating IL-11 production in cells which express IL-11Rα and gp130.

The IC₅₀ for an agent for inhibition of IL-11-mediated signalling may be determined, e.g. by culturing Ba/F3 cells expressing IL-11Rα and gp130 in the presence of human IL-11 and the agent, and measuring 3H-thymidine incorporation into DNA. In some embodiments, the agent may exhibit an IC₅₀ of 10 μg/ml or less, preferably one of ≤5 μg/ml, ≤4 μg/ml, ≤3.5 μg/ml, ≤3 μg/ml, ≤2 μg/ml, ≤1 μg/ml, ≤0.9 μg/ml, ≤0.8 μg/ml, ≤0.7 μg/ml, ≤0.6 μg/ml, or ≤0.5 μg/ml in such an assay.

In some embodiments, the IL-11-mediated signalling may be signalling mediated by binding of IL-11:IL-11Rα complex to gp130. In some embodiments, the IL-11:IL-11Rα complex may be soluble, e.g. complex of extracellular domain of IL-11Rα and IL-11, or complex of soluble IL-11Rα isoform/fragment and IL-11. In some embodiments, the soluble IL-11Rα is a soluble (secreted) isoform of IL-11Rα, or is the liberated product of proteolytic cleavage of the extracellular domain of cell membrane bound IL-11Rα.

In some embodiments, the IL-11:IL-11Rα complex may be cell-bound, e.g. complex of cell-membrane bound IL-11Rα and IL-11. Signalling mediated by binding of IL-11:IL-11Rα complex to gp130 can be analysed by treating cells expressing gp130 with IL-11:IL-11Rα complex, e.g. recombinant fusion protein comprising IL-11 joined by a peptide linker to the extracellular domain of IL-11Rα, e.g. hyper IL-11. Hyper IL-11 was constructed using fragments of IL-11Rα (amino acid residues 1 to 317 consisting of domain 1 to 3; UniProtKB: Q14626) and IL-11 (amino acid residues 22 to 199 of UniProtKB: P20809) with a 20 amino acid long linker (SEQ ID NO:20). The amino acid sequence for Hyper IL-11 is shown in SEQ ID NO:21.

In some embodiments, the agent may be capable of inhibiting signalling mediated by binding of IL-11:IL-11Rα complex to gp130, and is also capable of inhibiting signalling mediated by binding of IL-11 to IL-11Rα:gp130 receptor.

In some embodiments, the agent may be capable of inhibiting a process mediated by IL-11.

In some embodiments, the agent may be capable of inhibiting gene/protein expression of IL-11 and/or IL-11Rα. Gene and/or protein expression can be measured as described herein or by methods in the art that will be well known to a skilled person.

In some embodiments, the agent may be capable of inhibiting gene/protein expression of IL-11 and/or IL-11Rα to less than 100%, e.g. one of 99% or less, 95% or less, 90% or less, 85% or less, 80% or less, 75% or less, 70% or less, 65% or less, 60% or less, 55% or less, 50% or less, 45% or less, 40% or less, 35% or less, 30% or less, 25% or less, 20% or less, 15% or less, 10% or less, 5% or less, or 1% or less of the level of expression in the absence of the agent (or in the presence of an appropriate control agent). In some embodiments, the agent is capable of inhibiting gene/protein expression of IL-11 and/or IL-11Rα to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times the level of expression in the absence of the agent (or in the presence of an appropriate control agent).

### Treatment/Prevention of Kidney Injury

The present invention provides methods and articles (agents and compositions) for the treatment/prevention of kidney injury, e.g. as described herein and disorders, diseases or conditions associated with kidney injury.

Treatment is achieved by inhibition of IL-11-mediating signalling (i.e. antagonism of IL-11-mediated signalling). That is, the present invention provides for the treatment/prevention of kidney injury and disorders, diseases and conditions associated with kidney injury through inhibition of IL-11 mediated signalling, in e.g. a cell, tissue/organ/organ system/subject. In some embodiments, inhibition of IL-11-mediated signalling in accordance with the present disclosure comprises inhibition of IL-11-mediated signalling in cells of the kidney (e.g. tubule epithelial cells).

Provided is an agent capable of inhibiting interleukin 11 (IL-11)-mediated signalling for use in a method of treating or preventing kidney injury and/or a disorder, disease or condition associated with kidney injury.

Also provided is use of an agent capable of inhibiting interleukin 11 (IL-11)-mediated signalling for use in the manufacture of a medicament for use in a method of treating or preventing kidney injury and/or a disorder, disease or condition associated with kidney injury.

Further provided is a method of treating or preventing kidney injury and/or a disorder, disease or condition associated with kidney injury, the method comprising administering to a subject in need of treatment a therapeutically effective amount of an agent capable of inhibiting interleukin 11 (IL-11)-mediated signalling.

In some embodiments, the present invention provides for the treatment/prevention of kidney injury-related pathology in a disease/condition. That is, the present invention provides for the treatment/prevention of a disease/condition in which kidney injury is pathologically implicated. Kidney injury-related pathology is described herein. In particular, relevant pathology includes damage to tubular epithelial cells, either proximal, distal or both.

Agents capable of inhibiting interleukin 11 (IL-11)-mediated signalling are moreover demonstrated herein to be capable of reversing kidney injury. That is, inhibition of IL-11 mediated signalling is shown to be able to improve renal function following kidney injury.

Accordingly, the present invention contemplates to employ antagonists of IL-11 mediated signalling to enhance/improve renal function in subjects having impaired renal function, e.g. as a consequence of kidney injury.

Agents capable of inhibiting interleukin 11 (IL-11)-mediated signalling are useful for promoting the proliferation TECs, for the generation of functional, renal tissue. Thus agents capable of inhibiting interleukin 11 (IL-11)-mediated signalling are provided herein for promoting the proliferation, survival and/or function of tubular epithelial cells (TECs), and/or the growth, maintenance and/or function of renal tissue.

Agents capable of inhibiting interleukin 11 (IL-11)-mediated signalling are provided herein for regenerating tubular epithelial cells (TECs), and/or renal tissue.

The epithelialand/or acta to mesenchymal cell phenotype transition (also referred to herein as ‘EMT’) by tubular epithelial cells is associated with reduced kidney function. EMT by TECs is induced by soluble factors produced following tissue injury, such as TGFB1. Antagonism of IL-11 mediated signalling is shown herein to inhibit the EMT by TECs, and thereby preserve/improve renal function.

Expression of SNAIL is implicated in the loss of the ability of TECs to proliferate, and EMT. IL-11-mediated signalling is demonstrated herein to have a central role in upregulating SNAIL expression. Accordingly, the present invention contemplates to employ antagonists of IL-11 mediated signalling to inhibit SNAIL expression, e.g. in TECs. Antagonism of IL-11-mediated signalling releases TECs from SNAIL-mediated inhibition of TEC proliferation.

Accordingly, the present invention contemplates to employ antagonists of IL-11 mediated signalling to preserve/increase the number/proportion of cells in the kidney having a TEC phenotype. TEC phenotype may be characterised e.g. by E-cadherin expression. The present invention contemplates to employ antagonists of IL-11 mediated signalling to reduce the number/proportion of cells in the kidney having a mesenchymal cell-like phenotype. A mesenchymal cell-like phenotype may be characterised e.g. by SNAIL and/or ACTA2 expression, optionally with lack of E-cadherin expression.

Antagonists of IL-11 mediated signalling may be used in methods to preserve/increase the level of a function of TECs or renal tissue. TEC/renal tissue function may be evaluated e.g. by evaluation of a correlate of such activity. For example, TEC/renal tissue function may be evaluated by analysing the level of urea and/or creatine in the serum/blood (e.g. blood urea nitrogen), or by monitoring the albumin to creatine ratio (ACR). Antagonists of IL-11 mediated signalling may be used in methods to reduce the level of creatine and/or urea in the serum/blood, or to reduce ACR.

It will be clear to the person skilled in the art that the therapeutic and prophylactic utility of the present invention extends to essentially any disease/condition which would benefit from a reduction in kidney injury and/or kidney injury-related pathology. The therapeutic and prophylactic utility of the present invention extends to any subject suffering from kidney injury and/or a disorder, disease or condition associated with kidney injury. The therapeutic and prophylactic utility of the present invention also extends to any subject suffering from a disease in which kidney injury-related pathology is present.

In some embodiments, the present invention provides for the treatment/prevention of diseases/conditions that are caused/exacerbated by kidney injury. In some embodiments, there is provided the treatment/prevention of diseases/conditions in a subject in which kidney injury provides a poor prognosis.

Diagnosis and management of acute kidney injury is described in Rahman M et al., Acute Kidney injury: a guide to diagnosis and management. Am Fam Physician 2012 Oct. 1: 86(7):631-9. As described therein, acute kidney injury is characterized by abrupt deterioration in kidney function, manifested by an increase in serum creatinine level with or without reduced urine output.

In some embodiments, kidney injury and/or a disorder, disease or condition associated with kidney injury to be treated/prevented may be characterised by an increase in one or more of the following in an organ/tissue/subject affected by kidney injury and/or a disorder, disease or condition associated with kidney injury e.g. as compared to normal organ/lissue/subject (i.e. without kidney injury or a disorder, disease or condition associated with kidney injury): expression of one or more of IL-11, and IL-11Rα.

In some embodiments, the present invention provides for the treatment/prevention of kidney injury in the context of a disease/disorder/condition associated with kidney injury e.g. as described herein. In some embodiments, the present invention provides for the treatment/prevention of kidney injury and an underlying disease/disorder/condition associated with kidney injury. For example, inhibition of IL-11-mediated signalling has utility in antagonising the role of IL-11 in chemotherapy-associated kidney injury, as well as antagonising the role of IL-11 in the cancer itself.

Treatment/prevention of kidney injury and/or a disorder, disease or condition associated with kidney injury according to the present invention may be of kidney injury and/or a disorder, disease or condition associated with kidney injury that is associated with an upregulation of IL-11, e.g. an upregulation of IL-11 in cells or tissue in which the symptoms of the disease/disorder/condition manifests or may occur, or upregulation of extracellular IL-11 or IL-11Rα.

The disorder, disease or condition associated with kidney injury may affect any tissue or organ or organ system. In some embodiments, the disease/disorder/condition may affect several tissues/organs/organ systems. In some embodiments, the disease/disorder/condition affects the kidney.

In some embodiments, the disorder, disease or condition associated with kidney injury affects one or more of: the cardiovascular system, the digestive system, the excretory system, the respiratory system, the renal system, the reproductive system, the circulatory system, the muscular system, the endocrine system, the exocrine system, the lymphatic system, the immune system, the nervous system, and/or the skeletal system.

In some embodiments, the present invention provides for the treatment/prevention of kidney injury-related pathology in acute kidney injury, nephrotoxicity, drug-induced kidney injury, drug-induced acute kidney injury, drug-induced nephrotoxicity, cisplatin-induced kidney injury, cisplatin-induced acute kidney injury, cisplatin-induced nephrotoxicity.

Treatment may be effective to prevent progression of kidney injury and/or a disorder, disease or condition associated with kidney injury, e.g. to reduce/delay/prevent worsening of, or to reduce/delay/prevent development of, kidney injury and/or a disorder, disease or condition associated with kidney injury. In some embodiments treatment may lead to an improvement, e.g. a reduction in the severity of, and/or a reversal of, the symptoms of kidney injury and/or a disorder, disease or condition associated with kidney injury. In some embodiments treatment may increase survival. In some embodiments treatment is effective to reverse the effects and/or symptoms of kidney injury and/or a disorder, disease or condition associated with kidney injury.

Prevention may refer to prevention of development of kidney injury and/or a disorder, disease or condition associated with kidney injury, and/or prevention of worsening of kidney injury and/or a disorder, disease or condition associated with kidney injury, e.g. prevention of progression of kidney injury and/or a disorder, disease or condition associated with kidney injury to a later or chronic stage.

In some embodiments, the present invention provides for the treatment/prevention of acute kidney injury (AKI), acute kidney failure, acute kidney disease, chronic kidney disease, kidney damage, drug-induced kidney injury, tubular necrosis, acute tubular necrosis, autoimmune kidney injury and cancer.

Acute tubular necrosis is the most common type of intrinsic acute kidney injury in hospitalized patients (Rahman M et al supra). The cause is often ischemic (from prolonged hypotension) or nephrotoxic (from an agent that is toxic to the tubular cells). Acute kidney injury caused by acute tubular necrosis often does not improve with adequate repletion of intravascular volume and blood flow to the kidneys. Both ischemic and nephrotoxic acute tubular necrosis can resolve over time, although temporary renal replacement therapy may be required, depending on the degree of renal injury and the presence of preexisting chronic kidney disease.

A “cancer” as referred to herein may be any unwanted cell proliferation (or any disease manifesting itself by unwanted cell proliferation), neoplasm or tumour or increased risk of or predisposition to the unwanted cell proliferation, neoplasm or tumour. The cancer may be benign or malignant and may be primary or secondary (metastatic). A neoplasm or tumour may be any abnormal growth or proliferation of cells and may be located in any tissue. Examples of tissues include the adrenal gland, adrenal medulla, anus, appendix, bladder, blood, bone, bone marrow, brain, breast, cecum, central nervous system (including or excluding the brain) cerebellum, cervix, colon, duodenum, endometrium, epithelial cells (e.g. renal epithelia), gallbladder, oesophagus, glial cells, heart, ileum, jejunum, kidney, lacrimal glad, larynx, liver, lung, lymph, lymph node (including abdominal lymph node, axillary lymph node, cervical lymph node, inguinal lymph node, mediastinal lymph node, pelvic lymph node, periaortic lymph node), lymphoblast, maxilla, mediastinum, mesentery, myometrium, nasopharynx, omentume, oral cavity, ovary, pancreas, parotid gland, peripheral nervous system peritoneum, pleura, prostate, salivary gland, sigmoid colon, skin, small intestine, soft tissues, spleen, stomach, testis, thymus, thyroid gland, tongue, tonsil, trachea, uterus, vulva, white blood cells.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

A “cancer” as used herein can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

In accordance with various aspects of the present invention, the methods may comprise one or more of the following (e.g. in the context of kidney injury):
Reducing necrosis of renal tissue and/or renal cells;
Reducing fibrosis of the kidney and/or renal tissue;
Reducing collagen content of, and/or inhibiting collagen deposition in, the kindey and/or renal tissue;
Increasing/maintaining renal function;
Increasing/maintaining urine output;
Reducing urinary albumin/creatinine ratio;
Reducing serum creatinine level;
Reducing serum urea level;
Reducing serum TGFβ1 level;
Increasing/maintaining kidney weight;
Increasing/maintaining renal cortical volume;
Increasing/maintaining body weight;
Inhibiting epithelial-to-mesenchymal cell transition of tubule epithelial cells;
Reducing the number/proportion of ACTA2+ve cells in the kidney:
Reducing SNAIL expression by renal cells and/or in the kidney/renal tissue; and
Increasing/maintaining E-cadherin expression by renal cells and/or in the kidney/renal tissue.

### Administration

Administration of an agent capable of inhibiting IL-11-mediated signalling is preferably in a “therapeutically effective” or “prophylactically effective” amount, this being sufficient to show benefit to the subject.

In some embodiments, the agent may be administered before, in conjunction with, or after the cause of the kidney injury, e.g. administration or consumption of a nephrotoxic medicine or exposure to an environmental source of kidney injury.

The actual amount administered, and rate and time-course of administration, will depend on the nature and severity of the kidney injury and the nature of the agent. Prescription of treatment, e.g. decisions on dosage etc., is within the responsibility of general practitioners and other medical doctors, and typically takes account of the disease/condition to be treated, the condition of the individual subject, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

Multiple doses of the agent may be provided. One or more, or each, of the doses may be accompanied by simultaneous or sequential administration of another therapeutic agent.

Multiple doses may be separated by a predetermined time interval, which may be selected to be one of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

In therapeutic applications, agents capable of inhibiting IL-11-mediated signalling are preferably formulated as a medicament or pharmaceutical together with one or more other pharmaceutically acceptable ingredients well known to those skilled in the art, including, but not limited to, pharmaceutically acceptable carriers, adjuvants, excipients, diluents, fillers, buffers, preservatives, anti-oxidants, lubricants, stabilisers, solubilisers, surfactants (e.g., wetting agents), masking agents, colouring agents, flavouring agents, and sweetening agents.

The term “pharmaceutically acceptable” as used herein pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio.

Each carrier, adjuvant, excipient, etc. must also be “acceptable” in the sense of being compatible with the other ingredients of the formulation.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulations may be prepared for topical, parenteral, systemic, intravenous, intra-arterial, intramuscular, intrathecal, intraocular, intra-conjunctival, subcutaneous, oral or transdermal routes of administration which may include injection. Injectable formulations may comprise the selected agent in a sterile or isotonic medium. The formulation and mode of administration may be selected according to the agent and disease/disorder/condition to be treated.

In some cases, an article (e.g. agent/composition) as described herein is administered for treatment as described herein in conjunction with treatment for a disease/disorder/condition associated with kidney injury. Suitable treatments for a disease/disorder/condition associated with kidney injury are known in the art. A composition may be administered alone or in combination with other treatments, either simultaneously or sequentially dependent upon the disease/disorder/condition to be treated. For example, the article may be administered before, at the same time as, or after the treatment. The article and the treatment may be formulated together, e.g. in a formulation described above, or formulated separately.

### Detection of IL-11 and Receptors for IL-11

Some aspects and embodiments of the present invention concern detection of expression of IL-11 or a receptor for IL-11 (e.g. IL-11Rα, gp130, or a complex containing IL-11Rα and/or gp130) in a sample obtained from a subject.

In some aspects and embodiments the present invention concerns the upregulation of expression (over-expression) of IL-11 or a receptor for IL-11 (as a protein or oligonucleotide encoding the respective IL-11 or receptor for IL-11) and detection of such upregulation as an indicator of suitability for treatment with an agent capable of inhibiting the action of IL-11 or with an agent capable of preventing or reducing the expression of IL-11 or a receptor for IL-11.

Upregulated expression comprises expression at a level that is greater than would normally be expected for a cell or tissue of a given type. Upregulation may be determined by measuring the level of expression of the relevant factor in a cell or tissue. Comparison may be made between the level of expression in a cell or tissue sample from a subject and a reference level of expression for the relevant factor, e.g. a value or range of values representing a normal level of expression of the relevant factor for the same or corresponding cell or tissue type. In some embodiments reference levels may be determined by detecting expression of IL-11 or a receptor for IL-11 in a control sample, e.g. in corresponding cells or tissue from a healthy subject or from healthy tissue of the same subject. In some embodiments reference levels may be obtained from a standard curve or data set.

Levels of expression may be quantitated for absolute comparison, or relative comparisons may be made.

In some embodiments upregulation of IL-11 or a receptor for IL-11 (e.g. IL-11Rα, gp130, or a complex containing IL-11Rα and/or gp130) may be considered to be present when the level of expression in the test sample is at least 1.1 times that of a reference level. More preferably, the level of expression may be selected from one of at least 1.2, at least 1.3, at least 1.4, at least 1.5, at least 1.6, at least 1.7, at least 1.8, at least 1.9, at least 2.0, at least 2.1, at least 2.2, at least 2.3, at least 2.4 at least 2.5, at least 2.6, at least 2.7, at least 2.8, at least 2.9, at least 3.0, at least 3.5, at least 4.0, at least 5.0, at least 6.0, at least 7.0, at least 8.0, at least 9.0, or at least 10.0 times that of the reference level.

Expression levels may be determined by one of a number of known in vitro assay techniques, such as POR based assays, in situ hybridisation assays, flow cytometry assays, immunological or immunohistochemical assays.

By way of example suitable techniques involve a method of detecting the level of IL-11 or a receptor for IL-11 in a sample by contacting the sample with an agent capable of binding IL-11 or a receptor for IL-11 and detecting the formation of a complex of the agent and IL-11 or receptor for IL-11. The agent may be any suitable binding molecule, e.g. an antibody, polypeptide, peptide, oligonucleotide, aptamer or small molecule, and may optionally be labelled to permit detection, e.g. visualisation, of the complexes formed. Suitable labels and means for their detection are well known to those in the art and include fluorescent labels (e.g. fluorescein, rhodamine, eosine and NDB, green fluorescent protein (GFP), chelates of rare earths such as europium (Eu), terbium (Tb) and samarium (Sm), tetramethyl rhodamine, Texas Red, 4-methyl umbelliferone, 7-amino-4-methyl coumarin, Cy3, Cy5), isotope markers, radioisotopes (e.g. 32P, 33P, 35S), chemiluminescence labels (e.g. acridinium ester, luminol, isoluminol), enzymes (e.g. peroxidase, alkaline phosphatase, glucose oxidase, beta-galactosidase, luciferase), antibodies, ligands and receptors. Detection techniques are well known to those of skill in the art and can be selected to correspond with the labelling agent. Suitable techniques include PCR amplification of oligonucleotide tags, mass spectrometry, detection of fluorescence or colour, e.g. upon enzymatic conversion of a substrate by a reporter protein, or detection of radioactivity.

Assays may be configured to quantify the amount of IL-11 or receptor for IL-11 in a sample. Quantified amounts of IL-11 or receptor for IL-11 from a test sample may be compared with reference values, and the comparison used to determine whether the test sample contains an amount of IL-11 or receptor for IL-11 that is higher or lower than that of the reference value to a selected degree of statistical significance.

Quantification of detected IL-11 or receptor for IL-11 may be used to determine up- or down-regulation or amplification of genes encoding IL-11 or a receptor for IL-11. In cases where the test sample contains fibrotic cells, such up-regulation, down-regulation or amplification may be compared to a reference value to determine whether any statistically significant difference is present.

A sample obtained from a subject may be of any kind. A biological sample may be taken from any tissue or bodily fluid, e.g. a blood sample, blood-derived sample, serum sample, lymph sample, semen sample, saliva sample, synovial fluid sample. A blood-derived sample may be a selected fraction of a patient's blood, e.g. a selected cell-containing fraction or a plasma or serum fraction. A sample may comprise a tissue sample or biopsy; or cells isolated from a subject. Samples may be collected by known techniques, such as biopsy or needle aspirate. Samples may be stored and/or processed for subsequent determination of IL-11 expression levels.

Samples may be used to determine the upregulation of IL-11 or receptor for IL-11 in the subject from which the sample was taken.

In some preferred embodiments a sample may be a tissue sample, e.g. biopsy, taken from kidney tissue, cardiac tissue, visceral organ tissue, respiratory system organ tissue, or urinary/renal system tissue. A sample may contain cells.

A subject may be selected for therapy/prophylaxis in accordance with the present invention based on determination that the subject has an upregulated level of expression of IL-11 or of a receptor for IL-11 (e.g. IL-11Rα, gp130, or a complex containing IL-11Rα and/or gp130). Upregulated expression of IL-11 or of a receptor for IL-11 may serve as a marker of kidney injury and/or a disorder, disease or condition associated with kidney injury suitable for treatment with an agent capable of inhibiting IL-11 mediated signalling.

Upregulation may be in a given tissue or in selected cells from a given tissue. A preferred tissue may be kidney/renal tissue. Upregulation of expression of IL-11 or of a receptor for IL-11 may also be determined in a circulating fluid, e.g. blood, or in a blood derived sample. Upregulation may be of extracellular IL-11 or IL-11Rα. In some embodiments expression may be locally or systemically upregulated.

Following selection, a subject may be administered with an agent capable of inhibiting IL-11 mediated signalling.

### Diagnosis and Prognosis

Detection of upregulation of expression of IL-11 or a receptor for IL-11 (e.g. IL-11Rα, gp130, or a complex containing IL-11Rα and/or gp130) may also be used in a method of diagnosing kidney injury and/or a disorder, disease or condition associated with kidney injury, identifying a subject at risk of developing kidney injury and/or a disorder, disease or condition associated with kidney injury, and in methods of prognosing or predicting a subject's response to treatment with an agent capable of inhibiting IL-11 mediated signalling.

“Developing”, “development” and other forms of “develop” may refer to the onset of a disorder/disease, or the continuation or progression of a disorder/disease.

In some embodiments a subject may be suspected of having or suffering from kidney injury and/or a disorder, disease or condition associated with kidney injury, e.g. based on the presence of other symptoms indicative of kidney injury and/or a disorder, disease or condition associated with kidney injury in the subject's body or in selected cells/tissues of the subject's body, or be considered at risk of developing kidney injury and/or a disorder, disease or condition associated with kidney injury, e.g. because of genetic predisposition or exposure to environmental conditions, known to be risk factors for kidney injury and/or a disorder, disease or condition associated with kidney injury. Determination of upregulation of expression of IL-11 or a receptor for IL-11 may confirm a diagnosis or suspected diagnosis, or may confirm that the subject is at risk of developing kidney injury and/or a disorder, disease or condition associated with kidney injury. The determination may also diagnose kidney injury and/or a disorder, disease or condition associated with kidney injury or predisposition as one suitable for treatment with an agent capable of inhibiting IL-11-mediated signalling.

As such, a method of providing a prognosis for a subject having, or suspected of having kidney injury and/or a disorder, disease or condition associated with kidney injury may be provided, the method comprising determining whether the expression of IL-11 or a receptor for IL-11 is upregulated in a sample obtained from the subject and, based on the determination, providing a prognosis for treatment of the subject with an agent capable of inhibiting IL-11-mediated signalling.

In some aspects, methods of diagnosis or methods of prognosing or predicting a subject's response to treatment with an agent capable of inhibiting IL-11-mediated signalling may not require determination of the expression of IL-11 or a receptor for IL-11, but may be based on determining genetic factors in the subject that are predictive of upregulation of expression or activity. Such genetic factors may include the determination of genetic mutations, single nucleotide polymorphisms (SNPs) or gene amplification in IL-11, IL-11Rα and/or gp130 which are correlated with and/or predictive of upregulation of expression or activity and/or IL-11 mediated signalling. The use of genetic factors to predict predisposition to a disease state or response to treatment is known in the art, e.g. see Peter Stärkel Gut 2008; 57:440-442; Wright et al., Mol. Cell. Biol. March 2010 vol. 30 no. 6 1411-1420.

Genetic factors may be assayed by methods known to those of ordinary skill in the art, including POR based assays, e.g. quantitative PCR, competitive PCR. By determining the presence of genetic factors, e.g. in a sample obtained from a subject, a diagnosis may be confirmed, and/or a subject may be classified as being at risk of developing kidney injury and/or a disorder, disease or condition associated with kidney injury, and/or a subject may be identified as being suitable for treatment with an agent capable of inhibiting IL-11 mediated signalling.

Some methods may comprise determination of the presence of one or more SNPs linked to secretion of IL-11 or susceptibility to development of kidney injury and/or a disorder, disease or condition associated with kidney injury. SNPs are usually bi-allelic and therefore can be readily determined using one of a number of conventional assays known to those of skill in the art (e.g. see Anthony J. Brookes. The essence of SNPs. Gene Volume 234, Issue 2, 8 Jul. 1999, 177-186; Fan et al., Highly Parallel SNP Genotyping. Cold Spring Harb Symp Quant Biol 2003. 68:69-78; Matsuzaki et al., Parallel Genotyping of Over 10,000 SNPs using a one-primer assay on a high-density oligonucleotide array. Genome Res. 2004. 14:414-425).

The methods may comprise determining which SNP allele is present in a sample obtained from a subject. In some embodiments determining the presence of the minor allele may be associated with increased IL-11 secretion or susceptibility to development of kidney injury and/or a disorder, disease or condition associated with kidney injury.

Accordingly, in one aspect of the present invention a method for screening a subject is provided, the method comprising:
obtaining a nucleic acid sample from the subject;
determining which allele is present in the sample at the polymorphic nucleotide position of one or more of the SNPs listed in FIG. 33, FIG. 34, or FIG. 35 of WO 2017/103108 A1 (incorporated by reference herein), or a SNP in linkage disequilibrium with one of the listed SNPs with an r²≥0.8.

The determining step may comprise determining whether the minor allele is present in the sample at the selected polymorphic nucleotide position. It may comprise determining whether 0, 1 or 2 minor alleles are present.

The screening method may be, or form part of, a method for determining susceptibility of the subject to development of kidney injury and/or a disorder, disease or condition associated with kidney injury, or a method of diagnosis or prognosis as described herein.

The method may further comprise the step of identifying the subject as having susceptibility to, or an increased risk of, developing kidney injury and/or a disorder, disease or condition associated with kidney injury, e.g. if the subject is determined to have a minor allele at the polymorphic nucleotide position. The method may further comprise the step of selecting the subject for treatment with an agent capable of inhibiting IL-11 mediated signalling and/or administering an agent capable of inhibiting IL-11 mediated signalling to the subject in order to provide a treatment for kidney injury and/or a disorder, disease or condition associated with kidney injury in the subject or to prevent development or progression of kidney injury and/or a disorder, disease or condition associated with kidney injury in the subject.

In some embodiments, a method of diagnosing kidney injury and/or a disorder, disease or condition associated with kidney injury, identifying a subject at risk of developing kidney injury and/or a disorder, disease or condition associated with kidney injury, and methods of prognosing or predicting a subject's response to treatment with an agent capable of inhibiting IL-11 mediated signalling employs an indicator that is not detection of upregulation of expression of IL-11 or a receptor for IL-11, or genetic factors.

In some embodiments, a method of diagnosing kidney injury and/or a disorder, disease or condition associated with kidney injury, identifying a subject at risk of developing kidney injury and/or a disorder, disease or condition associated with kidney injury, and methods of prognosing or predicting a subject's response to treatment with an agent capable of inhibiting IL-11 mediated signalling is based on detecting, measuring and/or identifying one or more of the following indicators or performing one of the following analyses:
Elevated serum creatinine, blood urea and/or nitrogen
Urinary sodium and creatinine levels
Reduced urine output
Renal ultrasonography to identify presence/absence of tubular obstruction
Renal biopsy
Performing urinalysis.

Reference levels for laboratory kidney tests can be found in e.g. Rahman et al supra, which is hereby incorporated by reference in its entirety.

Methods of diagnosis or prognosis may be performed in vitro on a sample obtained from a subject, or following processing of a sample obtained from a subject. Once the sample is collected, the patient is not required to be present for the in vitro method of diagnosis or prognosis to be performed and therefore the method may be one which is not practised on the human or animal body. The sample obtained from a subject may be of any kind, as described herein above.

Other diagnostic or prognostic tests may be used in conjunction with those described here to enhance the accuracy of the diagnosis or prognosis or to confirm a result obtained by using the tests described here.

### Subjects

Subjects may be animal or human. Subjects are preferably mammalian, more preferably human. The subject may be a non-human mammal, but is more preferably human. The subject may be male or female. The subject may be a patient. The patient may have kidney injury and/or a disorder, disease or condition associated with kidney injury as described herein. A subject may have been diagnosed with kidney injury and/or a disorder, disease or condition associated with kidney injury requiring treatment, may be suspected of having such kidney injury and/or a disorder, disease or condition associated with kidney injury, or may be at risk from developing kidney injury and/or a disorder, disease or condition associated with kidney injury.

In embodiments according to the present invention the subject is preferably a human subject. In embodiments according to the present invention, a subject may be selected for treatment according to the methods based on characterisation for certain markers of kidney injury and/or a disorder, disease or condition associated with kidney injury.

In some embodiments the subject may be a subject who is being administered a drug or medicine in order to treat a disease, condition or disorder that may or may not be manifest in kidney tissue. The subject may have developed a drug-induced kidney injury as a consequence, e.g. side-effect, of such treatment. The subject may be selected for treatment or preventative therapy according to this invention on the basis of having developed such drug-induced kidney injury.

In some embodiments, the subject may have received treatment with a chemotherapeutic agent, or may be receiving treatment with a chemotherapeutic agent. The subject may have, be suspected of having, or be in recovery or remission from, cancer and administration of the chemotherapeutic agent may form part of the subject's treatment. Accordingly, the subject may be a subject having chemotherapeutic agent-induced kidney injury, chemotherapeutic agent-induced acute kidney injury or chemotherapeutic agent-induced nephrotoxicity.

In some preferred embodiments, the subject may have received treatment with cisplatin, or may be receiving treatment with cisplatin. The subject may have, be suspected of having, or be in recovery or remission from, cancer and administration of cisplatin may form part of the subject's treatment. Accordingly, the subject may be a subject having cisplatin-induced kidney injury, cisplatin-induced acute kidney injury or cisplatin-induced nephrotoxicity.

A subject may optionally be receiving intermittent or regular dialysis.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4)772-780 software. When using such software, the default parameters, e.g. for gap penalty and extension penalty, are preferably used.

Sequences

| SEQ | | |
|---|---|---|
| ID NO: | DESCRIPTION | SEQUENCE |
| 1 | Human IL-11 (UniProt | MNCVCRLVLVVLSLWPDTAVAPGPPPGPPRVSPDPRAELDSTVLLTRSLLADTRQLA |
| | P20809) | AQLRDKFPADGDHNLDSLPTLAMSAGALGALQLPGVLTRLRADLLSYLRHVQWLRRA |
| | | GGSSLKTLEPELGTLQARLDRLLRRLQLLMSRLALPQPPPDPPAPPLAPPSSAWGGI |
| | | RAAHAILGGLHLTLDWAVRGLLLLKTRL |
| 2 | Human gp130 | MLTLQTWLVQALFIFLTTESTGELLDPCGYISPESPVVQLHSNFTAVCVLKEKCMDY |
| | (UniProt P40189-1) | HFVNANYIVWKTNHFTIPKEQYTIINRTASSVTFTDIASLNIQLTCNILTFGQLEQN |
| | | VYGITIISGLPPEKPKNLSCIVNEGKKMRCEWDGGRETHLETNFTLKSEWATHKFAD |
| | | CKAKRDTPTSCTVDYSTVYFVNIEVWVEAENALGKVTSDHINFDPVYKVKPNPPHNL |
| | | SVINSEELSSILKLTWTNPSIKSVIILKYNIQYRTKDASTWSQIPPEDTASTRSSFT |
| | | VQDLKPFTEYVFRIRCMKEDGKGYWSDWSEEASGITYEDRPSKAPSFWYKIDPSHTQ |
| | | GYRTVQLVWKTLPPFEANGKILDYEVTLTRWKSHLQNYTVNATKLTVNLTNDRYLAT |
| | | LTVRNLVGKSDAAVLTIPACDFQATHPVMDLKAFPKDNMLWVEWTTPRESVKKYILE |
| | | WCVLSDKAPCITDWQQEDGTVHRTYLRGNLAESKCYLITVTPVYADGPGSPESIKAY |
| | | LKQAPPSKGPTVRTKKVGKNEAVLEWDQLPVDVQNGFIRNYTIFYRTIIGNETAVNV |
| | | DSSHTEYTLSSLTSDTLYMVRMAAYTDEGGKDGPEFTFTTPKFAQGEIEAIVVPVCL |
| | | AFLLTTLLGVLFCFNKRDLIKKHIWPNVPDPSKSHIAQWSPHTPPRHNFNSKDQMYS |
| | | DGNFTDVSVVEIEANDKKPFPEDLKSLDLFKKEKINTEGHSSGIGGSSCMSSSRPSI |
| | | SSSDENESSQNTSSTVQYSTVVHSGYRHQVPSVQVFSRSESTQPLLDSEERPEDLQL |
| | | VDHVDGGDGILPRQQYFKQNCSQHESSPDISHFERSKQVSSVNEEDFVRLKQQISDH |
| | | ISQSCGSGQMKMFQEVSAADAFGPGTEGQVERFETVGMEAATDEGMPKSYLPQTVRQ |
| | | GGYMPQ |
| 3 | Human IL11 RA | MSSSCSGLSRVLVAVATALVSASSPCPQAWGPPGVQYGQPGRSVKLCCPGVTAGDPV |
| | (UniProt Q14626) | SWFRDGEPKLLQGPDSGLGHELVLAQADSTDEGTYICQTLDGALGGTVTLQLGYPPA |
| | | RPVVSCQAADYENFSCTWSPSQISGLPTRYLTSYRKKTVLGADSQRRSPSTGPWPCP |
| | | QDPLGAARCWHGAEFWSQYRINVTEVNPLGASTRLLDVSLQSILRPDPPQGLRVESV |
| | | PGYPRRLRASWTYPASWPCQPHFLLKFRLQYRPAQHPAWSTVEPAGLEEVITDAVAG |
| | | LPHAVRVSARDFLDAGTWSTWSPEAWGTPSTGTIPKEIPAWGQLHTQPEVEPQVDSP |
| | | APPRPSLQPHPRLLDHRDSVEQVAVLASLGILSFLGLVAGALALGLWLRLRRGGKDG |
| | | SPKPGFLASVIPVDRRPGAPNL |
| 4 | siRNA target IL-11 | CCTTCCAAAGCCAGATCTT |
| 5 | siRNA target IL-11 | GCCTGGGCAGGAACATATA |
| 6 | siRNA target IL-11 | CCTGGGCAGGAACATATAT |
| 7 | siRNA target IL-11 | GGTTCATTATGGCTGTGTT |
| 8 | siRNA target IL-11Rα | GGACCATACCAAAGGAGAT |
| 9 | siRNA target IL-11Rα | GCGTCTTTGGGAATCCTTT |
| 10 | siRNA target IL-11Rα | GCAGGACAGTAGATCCCT |
| 11 | siRNA target IL-11Rα | GCTCAAGGAACGTGTGTAA |
| 12 | siRNA to IL-11 | CCUUCCAAAGCCAGAUCUUdTdT-AAGAUCUGGCUUUGGAAGGdTdT |
| | (NM_000641.3) | |
| 13 | siRNA to IL-11 | GCCUGGGCAGGAACAUAUAdTdT-UAUAUGUUCCUGCCCAGGCdTdT |
| | (NM_000641.3) | |
| 14 | siRNA to IL-11 | CCUGGGCAGGAACAUAUAUdTdT-AUAUAUGUUCCUGCCCAGGdTdT |
| | (NM_000641.3) | |
| 15 | siRNA to IL-11 | GGUUCAUUAUGGCUGUGUUdTdT-AACACAGCCAUAAUGAACCdTdT |
| | (NM_000641.3) | |
| 16 | siRNA to IL-11Rα | GGACCAUACCAAAGGAGAUdTdT-AUCUCCUUUGGUAUGGUCCdTdT |
| | (U32324.1) | |
| 17 | siRNA to IL-11Rα | GCGUCUUUGGGAAUCCUUUdTdT-AAAGGAUUCCCAAAGACGCdTdT |
| | (U32324.1) | |
| 18 | siRNA to IL-11Rα | GCAGGACAGUAGAUCCCUAdTdT-UAGGGAUCUACUGUCCUGCdTdT |
| | (U32324.1) | |
| 19 | siRNA to IL-11Rα | GCUCAAGGAACGUGUGUAAdTdT-UUACACACGUUCCUUGAGCdTdT |
| | (U32324.1) | |
| 20 | 20 amino acid linker | GPAGQSGGGGGSGGGSGGGSV |
| 21 | Hyper IL-11 (IL-11RA: | MSSSCSGLSRVLVAVATALVSASSPCPQAWGPPGVQYGQPGRSVKLCCPGVTAGDPV |
| | IL-11 fusion) | SWFRDGEPKLLQGPDSGLGHELVLAQADSTDEGTYICQTLDGALGGTVTLQLGYPPA |
| | | RPVVSCQAADYENFSCTWSPSQISGLPTRYLTSYRKKTVLGADSQRRSPSTGPWPCP |
| | | QDPLGAARCVVHGAEFWSQYRINVTEVNPLGASTRLLDVSLQSILRPDPPQGLRVES |
| | | VPGYPRRLRASWTYPASWPCQPHFLLKFRLQYRPAQHPAWSTVEPAGLEEVITDAVA |
| | | GLPHAVRVSARDFLDAGTWSTWSPEAWGTPSTGPAGQSGGGGGSGGGSGGGSVPGPP |
| | | PGPPRVSPDPRAELDSTVLLTRSLLADTRQLAAQLRDKFPADGDHNLDSLPTLAMSA |
| | | GALGALQLPGVLTRLRADLLSYLRHVQWLRRAGGSSLKTLEPELGTLQARLDRLLRR |
| | | LQLLMSRLALPQPPPDPPAPPLAPPSSAWGGIRAAHAILGGLHLTLDWAVRGLLLLK |
| | | TRL |
| 22 | Enx203 VH | EVQLQQSGPELVKPGASVKIPCKASGYTFTDYNMDWVKQSHGKSLEWIGDINPHNGG |
| | | PIYNQKFTGKATLTVDKSSSTAYMELRSLTSEDTAVYYCARGELGHWYFDVWGTGTT |
| | | VTVSS |
| 23 | Enx203 VL | DIVLTQSPASLAVSLGQRATISCRASKSVSTSGYSYIHWYQQKPGQPPKLLIYLASN |
| | | LDSGVPARFSGSGSGTDFTLNIHPVEEEDAATYYCQHSRDLPPTFGGGTKLEIK |
| 24 | Enx209 VH | QVQLQQPGAELVRPGSSVKLSCKASGYTFTNYWMHWLKQRPVQGLEWIGNIGPSDSK |
| | | THYNQKFKDKATLTVDKSSSTAYMQLNSLTSEDSAVYYCARGDYVLFTYWGQGTLVT |
| | | VSA |
| 25 | Enx209 VL | DIVLTQSPATLSLSPGERATLSCRASQSISNNLHWYQQKSHEAPRLLIYASQSISGI |
| | | PARFSGSGSGTDFTLSFSSLETEDFAVYFCQQSYSWPLTFGQGTKLEIK |
| 26 | Enx108A VH | QVQLVQSGGGWQPGRSLRLSCAASGFTFSSYGMHWVRQAPGKGLEWVAVISYDGSNK |
| | | YYADSVKGRFTISRDNSKNTLYLQMNSLRAEDTAVYYCAKIGATDPLDYWGQGTLVT |
| | | VSS |
| 27 | Enx108.A VL | QSALTQPRSVSGSPGQSVTLSCTGTSSDVGGYNYVSWYQHYPGKAPKLMIFDVNERS |
| | | SGVPDRFSGSKSGNTASLTiSGLQAEDEADYYCASYAGRYTWMFGGGTKVTVLG |
| 28 | EnxA hlgG4 | QVQLVQSGGGWQPGRSLRLSCAASGFTFSSYGMHWVRQAPGKGLEWVAVISYDGSNK |
| | (L248E, S241P) HC | YYADSVKGRFTISRDNSKNTLYLQMNSLRAEDTAVYYCAKIGATDPLDYWGQGTLVT |
| | | VSSASTKGPSVFPLAPCSRSTSESTAALGCLVKDYFPEPVTVSWNSGALTSGVHTFP |
| | | AVLQSSGLYSLSSVVTVPSSSLGTKTYTCNVDHKPSNTKVDKRVESKYGPPCPPCPA |
| | | PEFEGGPSVFLFPPKPKDTLMiSRTPEVTCVWDVSQEDPEVQFNWYVDGVEVHNAKT |
| | | KPREEQFNSTYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTPPVLDSDGS |
| | | FFLYSRLTVDKSRWQEGNVFSCSVMHEALHNHYTQKSLSLSLGK |
| 29 | Enx108A lambda LC | QSALTQPRSVSGSPGQSVTLSCTGTSSDVGGYNYVSWYQHYPGKAPKLMIFDVNERS |
| | | SGVPDRFSGSKSGNTASLTISGLQAEDEADYYCASYAGRYTWMFGGGTKVTVLGQPK |
| | | AAPSVILFPPSSEELQANKATLVCLISDFYPGAVTVAWKADSSPVKAGVETTTPSKQ |
| | | SNNKYAASSYLSLTPEQWKSHRSYSCQVTHEGSTVEKTVAPTECS |
| 30 | hEnx203 VH | EVQLVQSGAEVKKPGASVKISCKASGYTFTDYNMDWVKQAPGQRLEWIGDINPHNGG |
| | | PIYNQKFTGRATLTVDKSASTAYMELSSLRSEDTAVYYCARGELGHWYFDVWGQGTT |
| | | VTVSS |
| 31 | hEnx203 VL | DIVLTQSPASLALSPGERATLSCRASKSVSTSGYSYIHWYQQKPGQAPRLLIYLASN |
| | | LDSGVPARFSGSGSGTDFTLTISSLEEEDFATYYCQHSRDLPPTFGQGTKLEIK |
| 32 | hEnx209 VH | QVQLVQSGAEVKKPGASVKVSCKASGYTFTNYWMHWLRQRPGQGLEWIGNIGPSDSK |
| | | THYNQKFKDRVTMTVDKSTSTAYMELSSLRSEDTAVYYCARGDYVLFTYWGQGTLVT |
| | | VSS |
| 33 | hEnx209 VL | DIVLTQSPATLSLSPGERATLSCRASQSISNNLHWYQQKPGQAPRLLIKYASQSISG |
| | | IPARFSGSGSGTDFTLTISSLEPEDFAVYYCQQSYSWPLTFGQGTKLEIK |
| 34 | Enx108A VH CDR1 | SYGMH |
| 35 | Enx108A VH CDR2 | VISYDGSNKYYADSVKG |
| 36 | Enx108A VH CDR3 | IGATDPLDY |
| 37 | Enx108A VL CDR1 | TGTSSDVGGYNYVS |
| 38 | Enx108A VL CDR2 | DVNERSS |
| 39 | Enx108A VL CDR3 | ASYAGRYTWM |
| 40 | Enx203, hEnx203 VH | DYNMD |
| | CDR1 | |
| 41 | Enx203, hEnx203 VH | DINPHNGGPIYNQKFTG |
| | CDR2 | |
| 42 | Enx203, hEnx203 VH | GELGHWYFDV |
| | CDR3 | |
| 43 | Enx203, hEnx203 VL | RASKSVSTSGYSYIH |
| | CDR1 | |
| 44 | Enx203, hEnx203 VL | LASNLDS |
| | CDR2 | |
| 45 | Enx203, hEnx203 VL | QHSRDLPPT |
| | CDR3 | |
| 46 | Enx209, hEnx209 VH | NYWMH |
| | CDR1 | |
| 47 | Enx209, hEnx209 VH | NIGPSDSKTHYNQKFKD |
| | CDR2 | |
| 48 | Enx209, hEnx209 VH | GDYVLFTY |
| | CDR3 | |
| 49 | Enx209, hEnx209 VL | RASQSISNNLH |
| | CDR1 | |
| 50 | Enx209, hEnx209 VL | YASQSIS |
| | CDR2 | |
| 51 | Enx209, hEnx209 VL | QQSYSWPLT |
| | CDR3 | |
| 52 | Human IGHG1 | ASTKGPSVFPLAPSSKSTSGGTAALGCLVKDYFPEPVTVSWNSGALTSGVHTFPAVL |
| | constant (K214R, | QSSGLYSLSSVVTVPSSSLGTQTYICNVNHKPSNTKVDKRVEPKSCDKTHTCPPCPA |
| | D356E, L358M) | PELLGGPSVFLFPPKPKDTLMISRTPEVTCVVVDVSHEDPEVKFNWYVDGVEVHNAK |
| | | TKPREEQYNSTYRVVSVLTVLHQDWLNGKEYKCKVSNKALPAPIEKTISKAKGQPRE |
| | | PQVYTLPPSREEMTKNQVSLTCLVKGFYPSDIAVEWESNGQPENNYKTTPPVLDSDG |
| | | SFFLYSKLTVDKSRWQQGNVFSCSVMHEALHNHYTQKSLSLSPGK |
| 53 | Human IGHG4 constant | ASTKGPSVFPLAPCSRSTSESTAALGCLVKDYFPEPVTVSWNSGALTSGVHTFPAVL |
| | (L248E, S241P) | QSSGLYSLSSVVTVPSSSLGTKTYTCNVDHKPSNTKVDKRVESKYGPPCPPCPAPEF |
| | | EGGPSVFLFPPKPKDTLMISRTPEVTCWVDVSQEDPEVQFNWYVDGVEVHNAKTKPR |
| | | EEQFNSTYRVVSVLTVLHQDWLNGKEYKCKVSNKGLPSSIEKTISKAKGQPREPQVY |
| | | TLPPSQEEMTKNQVSLTCLVKGFYPSDIAVEWESNGQPENNYKTTPPVLDSDGSFFL |
| | | YSRLTVDKSRWQEGNVFSCSVMHEALHNHYTQKSLSLSLGK |
| 54 | Human IGKC | RTVAAPSVFIFPPSDEQLKSGTASVVCLLNNFYPREAKVQWKVDNALQSGNSQESVT |
| | constant | EQDSKDSTYSLSSTLTLSKADYEKHKVYACEVTHQGLSSPVTKSFNRGEC |
| 55 | Human IGLC2 | GQPKAAPSVTLFPPSSEELQANKATLVCLISDFYPGAVTVAWKADSSPVKAGVETTT |
| | constant | PSKQSNNKYAASSYLSLTPEQWKSHRSYSCQVTHEGSTVEKTVAPTECS |
| 56 | hEnx203 hlgG1 HC | EVQLVQSGAEVKKPGASVKISCKASGYTFTDYNMDWVKQAPGQRLEWIGDINPHNGG |
| | | PIYNQKFTGRATLTVDKSASTAYMELSSLRSEDTAVYYCARGELGHWYFDVWGQGTT |
| | | VTVSSASTKGPSVFPLAPSSKSTSGGTAALGCLVKDYFPEPVTVSWNSGALTSGVHT |
| | | FPAVLQSSGLYSLSSVVTVPSSSLGTQTYICNVNHKPSNTKVDKRVEPKSCDKTHTC |
| | | PPCPAPELLGGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALPAPIEKTISKAK |
| | | GQPREPQVYTLPPSREEMTKNQVSLTCLVKGFYPSDIAVEWESNGQPENNYKTTPPV |
| | | LDSDGSFFLYSKLTVDKSRWQQGNVFSCSVMHEALHNHYTQKSLSLSPGK |
| 57 | hEnx203 kappa LC | DIVLTQSPASLALSPGERATLSCRASKSVSTSGYSYIHWYQQKPGQAPRLLIYLASN |
| | | LDSGVPARFSGSGSGTDFTLTISSLEEEDFATYYCQHSRDLPPTFGQGTKLEIKRTV |
| | | AAPSVFIFPPSDEQLKSGTASVVCLLNNFYPREAKVQWKVDNALQSGNSQESVTEQD |
| | | SKDSTYSLSSTLTLSKADYEKHKVYAGEVTHQGLSSPVTKSFNRGEC |
| 58 | hEnx209 hlgG4 | QVQLVQSGAEVKKPGASVKVSCKASGYTFTNYWMHWLRQRPGQGLEWIGNIGPSDSK |
| | (L248E, S241P) HC | THYNQKFKDRVTMTVDKSTSTAYMELSSLRSEDTAVYYCARGDYVLFTYWGQGTLVT |
| | | VSSASTKGPSVFPLAPCSRSTSESTAALGCLVKDYFPEPVTVSWNSGALTSGVHTFP |
| | | AVLQSSGLYSLSSVVTVPSSSLGTKTYTCNVDHKPSNTKVDKRVESKYGPPCPPCPA |
| | | PEFEGGPSVFLFPPKPKDTLMISRTPEVTCVVVDVSQEDPEVQFNWYVDGVEVHNAK |
| | | TKPREEQFNSTYRVVSVLTVLHQDWLNGKEYKCKVSNKGLPSSIEKTISKAKGQPRE |
| | | PQVYTLPPSQEEMTKNQVSLTCLVKGFYPSDIAVEWESNGQPENNYKTTPPVLDSDG |
| | | SFFLYSRLTVDKSRWQEGNVFSCSVMHEALHNHYTQKSLSLSLGK |
| 59 | hEnx209 kappa LC | DIVLTQSPATLSLSPGERATLSCRASQSISNNLHWYQQKPGQAPRLLIKYASQSISG |
| | | IPARFSGSGSGTDFTLTISSLEPEDFAVYYCQQSYSWPLTFGQGTKLEIKRTVAAPS |
| | | VFIFPPSDEQLKSGTASVVCLLNNFYPREAKVQWKVDNALQSGNSQESVTEQDSKDS |
| | | TYSLSSTLTLSKADYEKHKVYACEVTHQGLSSPVTKSFNRGEC |

### ***

The invention includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

Methods disclosed herein may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

Where a nucleic acid sequence is disclosed herein, the reverse complement thereof is also expressly contemplated.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

Aspects and embodiments of the present invention will now be discussed with reference to the accompanying figures. Further aspects and embodiments will be apparent to those skilled in the art. All documents mentioned in this text are incorporated herein by reference. While the invention has been described in conjunction with the exemplary embodiments described below, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

### BRIEF DESCRIPTION OF THE FIGURES

Embodiments and experiments illustrating the principles of the invention will now be discussed with reference to the accompanying figures.

FIG. 1. Micrographs showing neutralising anti-IL-11 antibodies prevent progression from AKI to CKD as compared to IgG control antibody. Mice were subjected to folate induced kidney injury (a model of acute tubular necrosis (ATN) and secondary fibrosis) and administered either IgG control, Enx203 (anti-IL-11 antibody) or ENx209 (anti-IL-11RA antibody). Kidneys were harvested after 28 days and used for Masson's trichrome staining. Both antibodies were remarkably effective at preventing collagen deposition.

FIGS. 2A and 2B. Charts showing neutralising anti-IL-11 antibodies prevent AKI progression. Mice were subjected to folate induced kidney injury and administered either IgG control or anti-IL-11 antibody (Enx203, AB1) or anti-IL11RA antibody (ENx209, AB2). At 28 days, kidneys were collected for HPA collagen assay and serum urine (collected in metabolic cages) assessed for urinary albumin/creatinine ratios. Note—day 2 makers of kidney injury were similar between treatment groups showing equal levels of initial renal damage but mice receiving anti-IL-11 therapies largely recovered function whereas those receiving IgG less effectively recovered function. (2A) Shows renal collagen content, and (2B) shows urinary albumin/creatinine ratios at day 28 for mice subjected to different treatments.

FIG. 3. Graph and image showing dose-dependent effects of ENx203 and ENx209 on kidney injury responses and ERK activation. Animals were treated with folate and antibodies were injected from one hour before injury. Renal collagen content was assessed after 28 days. Animals were treated with either 10 mg/kg or 20 mg/kg biweekly with IgG control antibody. No difference in between groups was observed and animals from both groups are plotted together. The folate model of kidney injury is very severe (see FIG. 4). However, even in the context of the strong chemical stimulus, ENx203 at doses down to 5 mg/kg twice a week was highly effective (upper panel). Individuals points indicate biological replicates. Data is shown as mean±SD. Biweekly (biw.) and triweekly (triw.) injection of antibodies. Monitoring IL-11-mediated ERK activation in the fibrotic kidney as a read out of target engagement (lower panel), the inventors were able to show effects of ExN203 at 1 mg/kg twice a week.

FIGS. 4A to 4C. Graphs showing dose-dependent effects of ENx203 and ENx209 on (4A) serum creatinine, (4B) serum urea and (4C) serum TGFB1 levels. Animals were treated with folate and antibodies were injected from one hour before injury. Serum marker levels were assessed after 28 days. Animals were treated with either 10 mg/kg or 20 mg/kg biweekly with IgG control antibody. No difference in between groups was observed and animals from both groups are plotted together. Individual points indicate biological replicates. Data is shown as mean±SD. Biweekly (biw.) and triweekly (triw.) injection of antibodies.

FIGS. 5A to 5C. Graphs showing the effects of treatment with (5A) ENx108A (IgG1), (5B) ENx203 or (5C) ENx209 on AKI progression. Mice were treated with folate and from 1 day before were also treated with antibodies at given concentrations (biweekly). Renal collagen content was determined 21 days after kidney injury. Data points indicate individual animals and all data was generated in parallel, so individual antibodies can be compared with each other. Identical baseline and IgG control animals are plotted for each antibody to facilitate data interpretation. Data are mean+−s.d.; Points indicate animals per group. Two-tailed Dunnett's test, corrected P-values.

FIGS. 6A and 6B. Graphs showing that fibroblast-specific knock out of Il11ra in mice protects from AKI. Animals were treated with tamoxifen to delete Il11ra1 from Col1a1+ve cells prior to renal injury with folate. After 21 days (6A) collagen content was assessed using the HPA assay and (6B) kidney function was determined via serum urea levels. Animals were protected to similar levels as during antibody treatment, suggesting a central role of IL-11 activity on fibroblasts in the pathogenesis of renal injury and dysfunction. Sidak corrected P-value.

FIGS. 7A to 7F. Schematic, graphs, and image relating to the effect of anti-IL-11 therapy from day 3 post AKI. (7A) Schematic representation of the timing of folate administration and antibody treatment. (7B) Mice receiving anti-IL-11 therapy gain weight soon after receiving anti-IL-11 therapy. (7C) At the end of the study period mice receiving anti-IL-11 therapy had higher kidney weights, (7D) less renal collagen, (7E) normal urine outputs and (7F) improved gross morphology as compared to mice receiving IgG.

FIGS. 8A to 8F. Schematic, graphs, and image showing that ENx203 treatment reverses kidney injury and dysfunction and induces kidney regeneration. (8A) Schematic representation of the timing of folate administration and antibody treatment. The folate model of kidney injury was established, and animal were not treated the until 21 days after injury. This led to severe reduction of kidney function and increase in collagen content. Antibody treatment was then initiated, and analysis of kidney function by evaluation of serum urea levels and collagen content was performed after 3, 6, 9 and 12 weeks of treatment. This revealed a significant (8B) reduction in collagen content, and (8C) improvement in renal function in ENx203-treated animals compared to animals before the treatment at day 21. While kidneys contained less collagen, they still (8D) gained weight and (8E), (8F) looked less bumpy and had healthier histology (cross-section of kidney, 3 animals). P values are corrected for multiple testing and indicate differences between later time points with the FA 21d groups (Dunnett test). Data is shown as mean±SD.

FIG. 9. Images showing that ENx203 treatment reduced collagen content and promoted restoration of normal renal parenchyma and cortical volume. Masson's Trichrome stain of mid-section of kidneys from mice day 21 (D21) after folic acid (FA) injury or control mice and then in mice treated from D21 post FA injury with either IgG or anti-IL-11 for 3, 6, 9 or 12 weeks.

FIG. 10. Images showing higher magnification view of the renal cortex from images shown in FIG. 9. Masson's Trichrome stain of the cortex of kidneys from mice day 21 (D21) after folic acid (FA) injury or control mice and then in mice treated from D21 post FA injury with either IgG or anti-IL-11 for 3, 6, 9 or 12 weeks.

FIGS. 11A and 11B. Graphs showing that ENx203 treatment after established renal injury induces weight gain and leads to a 50% reversal in urinary ACR. The folate model of kidney injury was performed, and animals were not treated until 21 days after injury. Antibody treatment was then initiated, and animal weight was continually assessed. (11A) ENx203-treated animals started to regain weight upon initiation of treatment. (11B) Urinary albumin: creatinine ratio was reversed by ˜50% by 12 weeks of therapy as compared to starting levels at 3-weeks post injury.

FIGS. 12A and 12B. Graphs showing that TGFB1-driven partial epithelial-mesenchymal transformation of tubular epithelial cells is IL-11 dependent. Human primary tubule epithelial cells were stimulated with TGFB1, IL-11 (5 ng/ml, 24 h) or TGFB1 and antibody (2 μg/ml, 24 h). (12A) The percentage of ACTA2^{+ve} cells and (12B) collagen expression was monitored on the Operetta high-content imaging platform.

FIG. 13. Images relating to TGFB1-driven partial epithelial-mesenchymal transformation of tubular epithelial cells. Top, human primary tubule epithelial cells were stimulated with IL-11 (2.5-20 ng/ml, 24 h). Operetta high-content imaging was used to visualise actin stress fibre formation using rhodamine-phalloidin. Bottom, visual representation of collagen expression in cells.

FIGS. 14A and 14B. Images and graph showing that expression of the fundamentally important Snail factor by Tubular epithelial cells (TECs) is IL-11 dependent. Primary human TECs were stimulated with either IL-11 or TGFB in the presence of IgG or anti-IL-11 antibody (ENx203) and expression of the Snall or ACTA2 gene detected using the Operetta imaging platform. (14A) both IL-11 and TGFB induce partial EMT and expression of both ACTA2 and Snail. In the presence of anti-IL-11 antibody this effect was strongly inhibited in TGFB stimulated cells. (14B) quantification of ACTA+ve cells from the micrographs of FIG. 14A.

FIG. 15. Image showing that anti-IL-11 antibody treatment prevents Snail induction in primary human TECs. Western blot of primary human kidney tubular epithelial cells treated with TGFB in the presence or absence of anti-IL-11 antibody (ENx203) or with IL-11. Upper panel, induction of SNAIL, the master regulator of EMT, by TGFB (5n g/ml) is dependent on IL-11 signaling and IL-11 (5 ng/ml) alone is able to induce SNAIL expression. Lower panel, equal protein loading confirmed by GAPDH control protein.

FIG. 16. Image showing that E-cadherin expression in regenerating kidneys is increased with anti-IL-11 therapy. E-cadherin expression is the canonical marker of epithelial cell identity and expression of E-cadherin is lost when cells undergo EMT or partial EMT. In keeping with an effect of IL-11 driving TECs into a partial EMT (pEMT), Western blot of kidneys from mice D21 post folate injury and those treated with IgG for 12 weeks after injury had lower E-cadherin levels compared to controls and this effect was reversed with anti-IL-11 therapy.

FIGS. 17A and 17B. Graph and bar chart relating to the effect of anti-IL-11 antibody treatment in a Unilateral Ureter Obstruction (UUO) model of kidney injury. UUO was performed to induce physical renal injury responses in mice. Some animals were treated with ENx203 or IgG control antibody at day 4, 7 and 9 of treatment (20 mg/kg). (17A) Body weight of ENx203 and IgG control treated animals. (17B) Collagen content in the kidney was assessed using the HPA assay 10 days after the procedure. Animals treated with ENx203 therapeutic antibody had significantly reduced collagen content compared to animals treated with control antibody. Data is shown as mean±SD. Sidak corrected P-value.

FIGS. 18A to 18H. Schematic and graphs relating to the effect of anti-IL-11 antibody treatment in a cisplatin-induced model of kidney injury. (18A) Mice were administered cisplatin (7 mg/kg cisplatin), once weekly for 4 weeks. A control group was administered with saline. Mice were administered biweekly from week 1 by IP injection with 10 mg/kg X203 or an isotype-matched IgG control antibody. Mice were harvested for analysis of the kidneys after 8 weeks. (18B) Collagen content in the kidney as assessed by the HPA assay. (18C to 18H) RNA expression of (18C) Col3, (18D) Fibronectin, (18E) MMP2, (18F) TIMP, (18G) CCL5 and (18H) CCL2 as determined by qPCR. Graphs show fold change (FC) in expression relative to expression in saline-treated mice.

### EXAMPLES

In the following examples, the inventors demonstrate that anti-IL-11 therapy can ameliorate kidney injury through extensive regeneration and reversal of renal impairment in models of acute and chronic kidney disease.

### Example 1: Materials and Methods

### 1.1 Primary Human Renal Proximal Tubule Epithelial Cells

### Culture and Stimulation Conditions

Primary human renal proximal tubule epithelial cells (HRPTEC, PCS-400-010, ATCC) were grown and maintained at 37° C. and 5% CO₂ in complete renal epithelial cell growth medium (PCS-400-030 and PCS-400-040, ATCC). HRPTEC medium was renewed every 2-3 days and cells were passaged at 80% confluence using standard trypsinization techniques. All the experiments were carried out at P3 and cells were serum-starved for 16 hours prior to respective stimulations (24 hours) that were performed in serum-free renal epithelial cell media. Stimulated cells were compared to unstimulated cells that have been grown for the same duration under the same conditions (serum-free renal epithelial cell media), but without the stimuli.

### Operetta Phenotyping Assay

HRPTEC were seeded in 96-well CellCarrier plates (600550, PerkinElmer) at a density of 1×10⁴ cells per well. Cells were stimulated with the indicated concentrations of IL-11 (Genscript) or TGF-β1 (PHC 143B, Bio-Rad) with and without the presence of 2 μg/ml of either anti IL-11 (ENx203) or IgG isotype control antibodies for 24 hours. Following experimental conditions, cells were fixed in 4% paraformaldehyde (PFA, 28908, Thermo Fisher Scientific), permeabilized with 0.1% Triton X-100 (T8787, Sigma) in PBS. Non-specific binding sites were blocked 0.5% BSA (A7906, Sigma) and 0.1% Tween-20 (170-6531, Bio-Rad) in PBS. Cells were incubated overnight (4C) with primary antibodies (1:500) i.e. ACTA2 (ab7817, Abcam), Collagen I (34710, Abcam), SNAIL (ab180714, Abcam) and followed by incubation with the appropriate AlexaFluor488-conjugated secondary antibodies (Goat anti-mouse 488, ab150113 and Goat anti-Rabbit 488, ab150077, Abcam) for 1 hour (1:1000, RT, dark). Cells were counter-stained with Rhodamine-Phalloidin (R415, Thermo Fisher Scientific) and DAPI (1 μg/ml, D1306, Thermo Fisher Scientific) in blocking solution. Plates were scanned and images were collected with an Operetta high-content imaging system (1483, PerkinElmer). Each condition was assayed from at least two wells and a minimum of seven fields per well. The quantification of ACTA2₊ cells was done using Harmony software version 3.5.2. The measurement of Collagen I fluorescence intensity per area was performed with Columbus version 2.7.1.

### 1.2 Western Blot

Western blot was carried out on HRPTE total protein extracts. HRPTE were lysed in radioimmunoprecipitation assay (RIPA) buffer containing protease and phosphatase inhibitors (Thermo Scientifics), followed by centrifugation to clear the lysate. Protein concentrations were determined by Bradford assay (Bio-Rad). Equal amount of protein lysates were separated by SDS-PAGE, transferred to PVDF membrane, and subjected to immunoblot analysis for SNAIL (3879, CST) and GAPDH (2118, CST).

Proteins were visualized using the ECL detection system (Pierce) with anti-rabbit-HRP (7074, CST).

### 1.3 Animal Models

All animal procedures were approved and conducted in accordance with the SingHealth Institutional Animal Care and Use Committee (IACUC). All mice were provided food and water ad libitum.

### Mouse Model of Chemically Induced-Acute Kidney Injury

Kidney injury was induced by IP injection of folic acid (200 mg/kg) in vehicle (0.3M NaHCO₃) to 10 weeks old male mice; control mice were administered vehicle alone. Animals were sacrificed 28 days post-FA. Mice were intraperitoneally injected with anti-IL-11 antibody (ENx203), anti-IL-11Rα antibody (ENx209) or identical concentration of IgG isotype control. Durations of treatment and dosage of antibody therapies are outlined in the figures.

### Mouse Model of Surgically Induced-Acute Kidney Injury

Unilateral ureteral obstruction (UUO) surgeries were carried out on 12 weeks old male mice. Briefly, mice were anesthetized by IP injection of ketamine (100 mg/kg)/xylazine (10 mg/kg) and full depth of anaesthesia was accessed with the pedal reflex. Mice were then shaved on the left side of the abdomen. A vertical incision was made through the skin with a scalpel, a second incision was made through the peritoneum to reveal the kidney. Using forceps, the kidney was brought to the surface and the ureter was tied with surgical silk, twice, below the kidney. The ligated kidney was placed gently back into its correct anatomical position and sterile saline was added to replenish loss of fluid. The incisions were then sutured. Animals were post-operatively treated with antibiotic enrofloxacin (15 mg/kg, SC) and analgesic buprenorphine (0.1 mg/kg, SC) for three consecutive days.

### 1.4. Colorimetric Assays and ELISA

The levels of blood urea nitrogen (BUN) in mouse serum were measured using Urea Assay Kit (ab83362, Abcam). Urine albumin and creatinine levels were measured using Mouse Albumin ELISA kit (ab108792, Abcam) and Creatinine Assay Kit (ab204537, Abcam), respectively. Serum TGFβ1 levels were measured by ELISA. All ELISA and colorimetric assays were performed according to the manufacturer's protocol.

### 1.5 Histology

Kidney tissues were fixed for 48 h at RT in 10% neutral-buffered formalin (NBF), dehydrated, embedded in paraffin blocks and sectioned at 4 μm. Sections were then stained with Masson's Trichrome according to standard protocol and examined by light microscopy.

### Example 2: Analysis of the Effect of Inhibition of IL-11 Mediated Signalling in a Model of Chemically-Induced Kidney Injury

Kidney injury was chemically-induced in 10-12 week old littermate mice of similar weight by intraperitoneal (i.p.) injection of folic acid (180 mg kg⁻¹) in vehicle (0.3 M NaHCO₃); control mice were administered vehicle alone.

Enx203 (an antibody capable of binding to mouse IL-11 (and human IL-11) and inhibiting IL-11 mediated signalling) or Enx209 (an antibody capable of binding to mouse IL-11 (and human IL-11) and inhibiting IL-11 mediated signalling) were administered one day after folic acid treatment, and then 3 times per week at a dose of 20 mg/kg. Mice were euthanized 28 days post-injection.

Enx203 and Enx209 are antagonists of IL-11 mediated signalling. Enx203 is a mouse anti-mouse IL-11 IgG, and is described e.g. in Ng et al., Sci Transl Med. (2019) 11(511) pii: eaaw1237 (also published as Ng, et al., “IL-11 is a therapeutic target in idiopathic pulmonary fibrosis.” bioRxiv 336537; doi: https://doi.org/10.1101/336537). Enx203 is also referred to as “X203”. Enx203 comprises the VH region according to SEQ ID NO:92 of WO 2019/238882 A1 (SEQ ID NO:22 of the present disclosure), and the VL region according to SEQ ID NO:94 of WO 2019/238882 A1 (SEQ ID NO:23 of the present disclosure). Enx209 is a mouse anti-mouse IL-11Rα IgG, and is described e.g. in Widjaja et al., Gastroenterology (2019) 157(3):777-792 (also published as Widjaja, et al., “IL-11 neutralising therapies target hepatic stellate cell-induced liver inflammation and fibrosis in NASH.” bioRxiv 470062; doi:https://doi.org/10.1101/470062). Enx209 is also referred to as “X209”. Enx209 comprises the VH region according to SEQ ID NO:7 of WO 2019/238884 A1 (SEQ ID NO:24 of the present disclosure), and the VL region according to SEQ ID NO:14 of WO 2019/238884 A1 (SEQ ID NO:25 of the present disclosure).

The mouse plasma levels of urea and creatinine were quantified using urea assay kit (ab83362, Abcam) and creatinine assay kit (ab65340, Abcam), respectively according to the manufacturer's instructions. The amount of total collagen in the kidney was quantified on the basis of colourimetric detection of hydroxyproline using a Quickzyme Total Collagen assay kit (Quickzyme Biosciences). All colourimetric assays were performed according to the manufacturer's instructions.

Tissues were paraffin-embedded, and kidneys were sectioned at 3 μm. For paraffin sections, tissues were fixed for 24 h, at room temperature in 10% neutral-buffered formalin (Sigma-Aldrich), dehydrated and embedded in paraffin. For cryosections, freshly dissected organs were embedded with Tissue-Tek Optimal Cutting Temperature compound (VWR International). Cryomoulds were then frozen in a metal beaker with isopentane cooled in liquid nitrogen and sections were stored in −80° C. Total collagen was stained with Masson's trichrome stain kit (HT15, Sigma-Aldrich) according to the manufacturer's instructions. Images of the sections were captured and blue-stained areas were semi-quantitatively determined with ImageJ software (version 1.49). For immunohistochemistry, the tissue sections were incubated with anti-ACTA2 antibody (ab5694, Abcam). Primary antibody staining was visualized using an ImmPRESS HRP Anti-Rabbit IgG Polymer Detection kit (Vector Laboratories) with ImmPACT DAB Peroxidase Substrate (Vector Laboratories) as the chromogen. The sections were then counterstained with Mayer's haematoxylin (Merck).

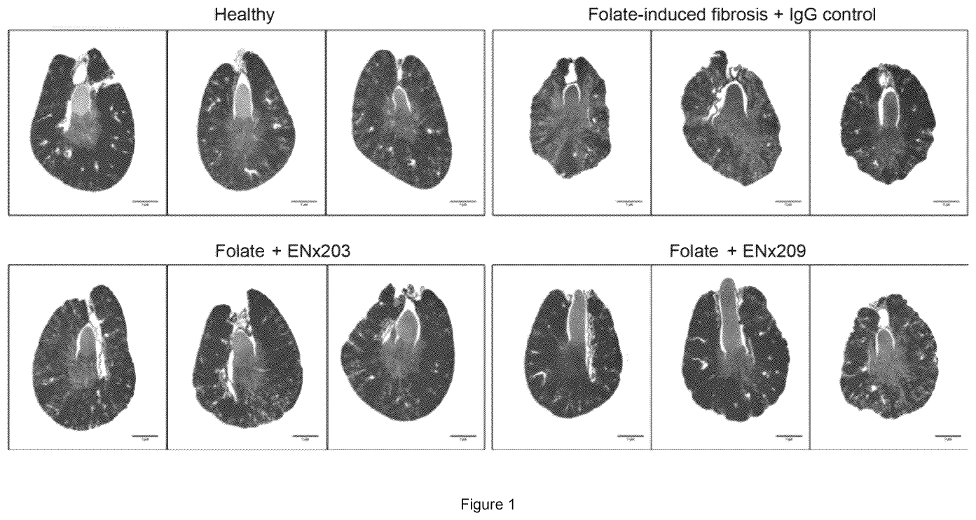

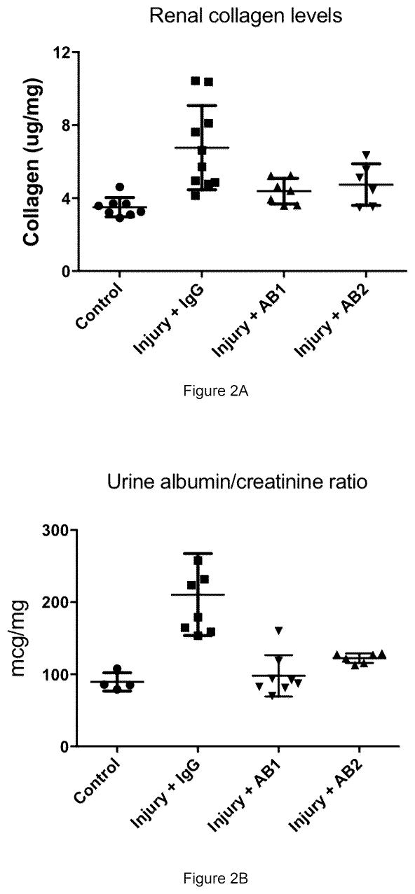

FIGS. 1 and 2 show that mice treated with anti-IL11 antibody or anti-IL-11Rα antibody antagonists were found to have significantly reduced staining for collagen.

FIG. 2 also shows that the urinary albumin/creatine ratio was significantly reduced by treatment with anti-IL-11 antibody or anti-IL-11Rα antibody, indicating a reduced level of kidney damage.

Importantly, the serum levels of urea and creatine at day 2 following chemical induction of kidney damage were similar between treatment groups (data not shown), however mice receiving antagonist IL-11 therapies largely recovered function whereas those receiving IgG less effectively recovered function.

In a separate experiment, a similar folate-induced model of kidney injury was used to evaluate the therapeutic efficacy of antagonists of IL-11 mediated signalling to treat/prevent kidney injury. Mice were treated with folate as described above, and antibodies were administered from one hour prior to injury. Renal collagen content was assessed after 28 days. Animals were treated with 1 mg/kg Enx203 biweekly, 5 mg/kg Enx203 biweekly, 5 mg/kg Enx203 triweekly, 10 mg/kg Enx203 biweekly, 10 mg/kg Enx203 triweekly, 20 mg/kg Enx203 triweekly, 20 mg/kg Enx209 biweekly, or 10 mg/kg or 20 mg/kg IgG control antibody biweekly.

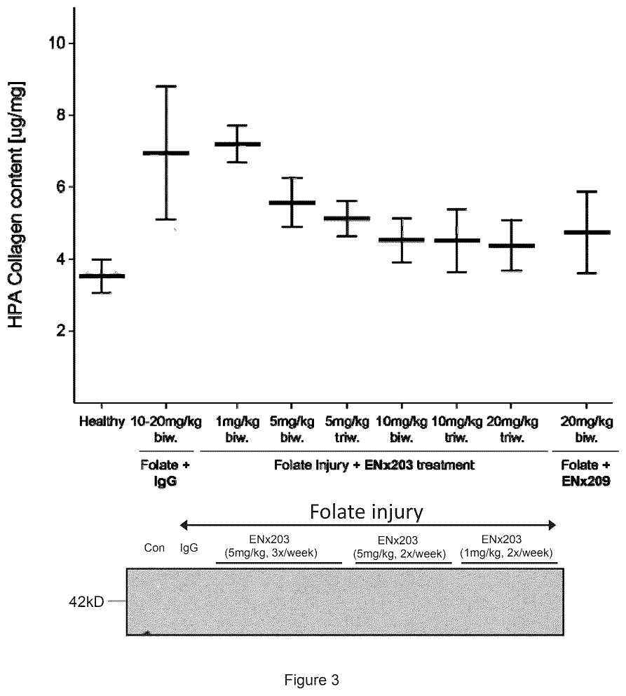

FIG. 3 shows that treatment with the antibody antagonists of IL-11-mediated signalling reduced the levels of collagen content associated with folic acid-induced kidney injury in a dose-dependent fashion. An effect on collagen levels was seen even when Enx203 was administered at 5 mg/kg, biweekly.

Monitoring IL-11-mediated ERK activation in the fibrotic kidney as a read out of target engagement, the inventors demonstrated an effect for ExN203 at a dose of 1 mg/kg, administered twice per week.

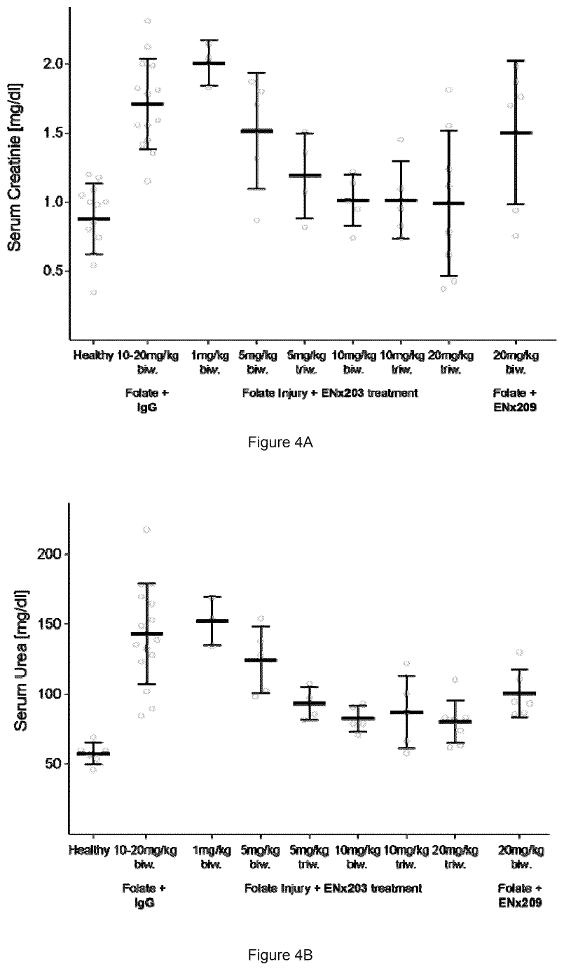

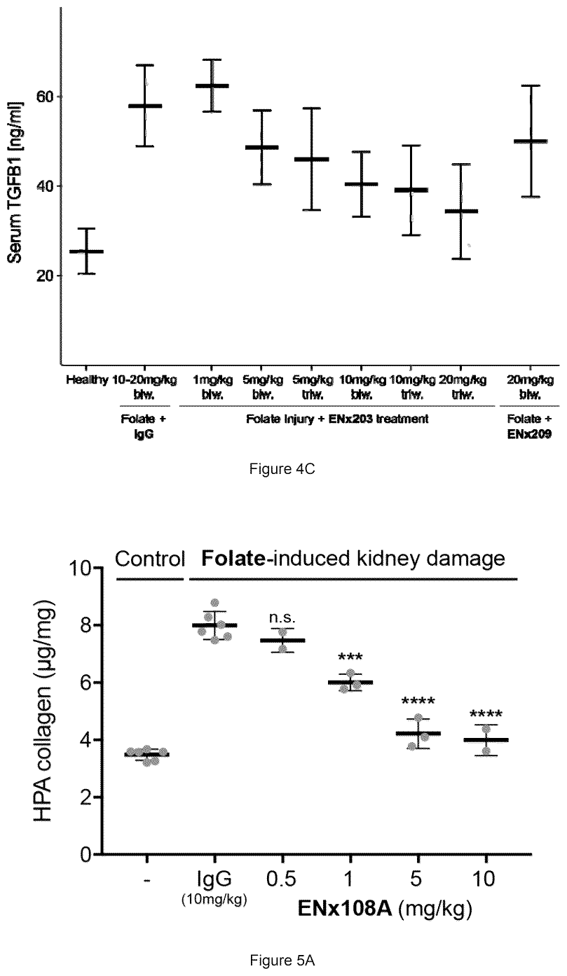

Serum levels of creatine, urea and TGFβ1 were also evaluated in these experiments, and FIG. 4 shows that treatment with the antibody antagonists of IL-11-mediated signalling reduced serum levels of these correlates of kidney injury in a dose-dependent manner. Again, reduction of the levels of these factors in serum was observed even when Enx203 was administered at 5 mg/kg, biweekly.

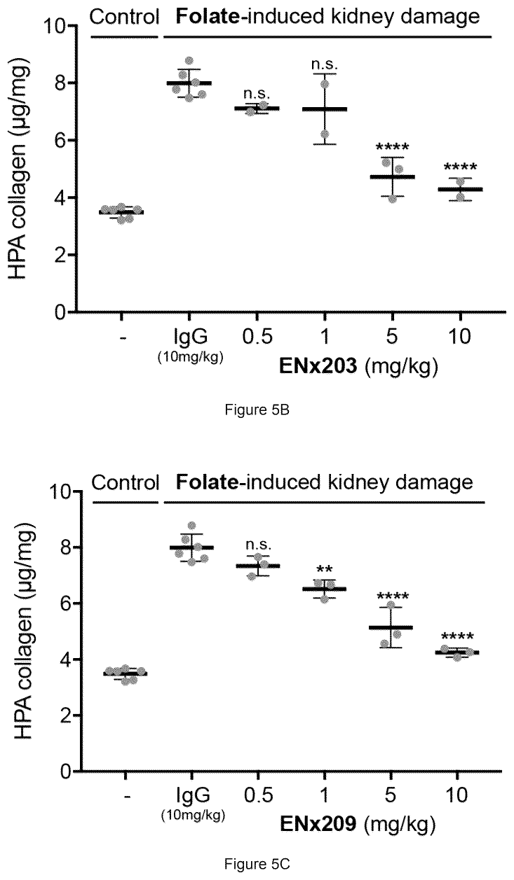

In further experiments, a similar folate-induced model of kidney injury was used to evaluate the therapeutic efficacy of antagonists of IL-11 mediated signalling to treat/prevent kidney injury. Mice were treated with folate as described above, and antibodies were administered from one day prior to injury. Renal collagen content was assessed after 21 days. Animals were administered biweekly with 10 mg/kg IgG control antibody, or 0.5 mg/kg, 1 mg/kg, 5 mg/kg or 10 mg/kg of Enx203, Enx209 or Enx108A. The results are shown in FIG. 5.

Enx108A is a human anti-human IL-11 IgG capable of binding to mouse IL-11 and human IL-11, and inhibiting IL-11 mediated signalling. Enx108A is described e.g. in WO 2019/238882 A1, and comprises the VH region according to SEQ ID NO:8 of WO 2019/238882 A1 (SEQ ID NO:26 of the present disclosure), and the VL region according to SEQ ID NO:20 of WO 2019/238882 A1 (SEQ ID NO:27 of the present disclosure). In the present Example, Enx108A is provided in higG4 (L248E, S241P), lambda light format (i.e. is formed of the heavy chain having the amino acid sequence shown in SEQ ID NO:28, and the light chain having the amino acid sequence shown in SEQ ID NO:29).

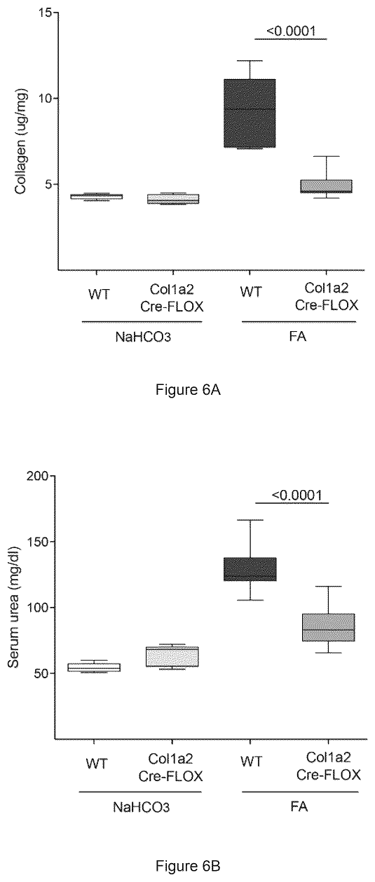

In a further experiment, the inventors investigated the consequences of IL11RA1 knockout in this model of acute, chemically-induced kidney injury. Cre-lox mice that display fibroblast-specific knockout of IL11RA1 in response to tamoxifen were treated with tamoxifen to delete IL-11RA1 from col1A2-positive cells, and subsequently subjected to folate-induced kidney injury as described above (or treatment with vehicle). After 21 days, collagen content was assessed using the HPA assay, and kidney function was determined by analysis of serum urea levels. The results are shown in FIG. 6. The IL11RA1 knockout in fibroblasts was found to protect the mice from folate-induced kidney injury, indicating a central role for IL-11 mediated signalling in fibroblasts in the pathology of renal injury and consequent dysfunction and secondary fibrosis.

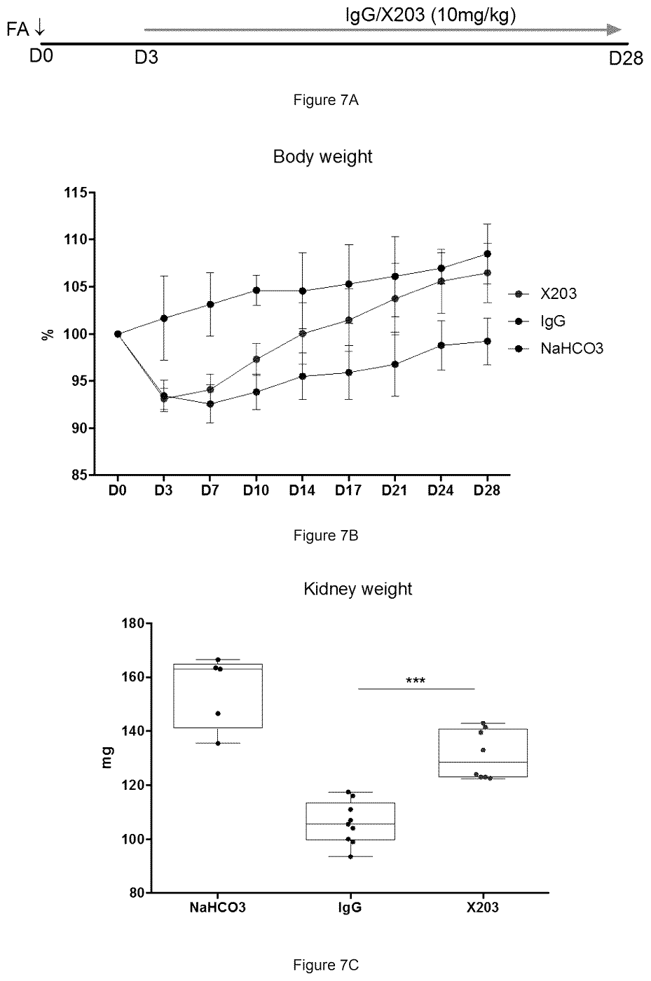

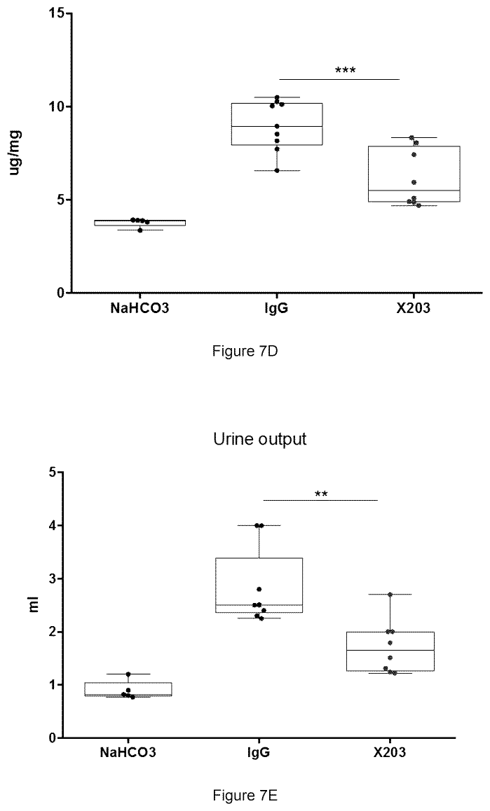

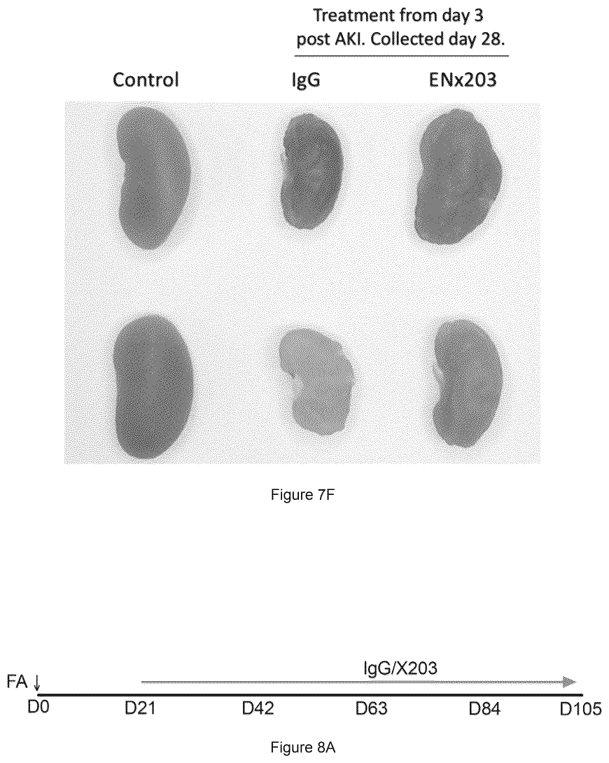

In another experiment, mice were treated with folate as described above, and Enx203 was administered from day 3, at 10 mg/kg, biweekly. Body weight, kidney weight, renal collagen content, urine output and gross kidney morphology was evaluated after 28 days. The results are shown in FIG. 7. At the end of the study, mice receiving anti-IL-11 antibody therapy had increased kidney weights, less renal collagen, normal urine outputs and improved gross morphology as compared to mice receiving IgG control.

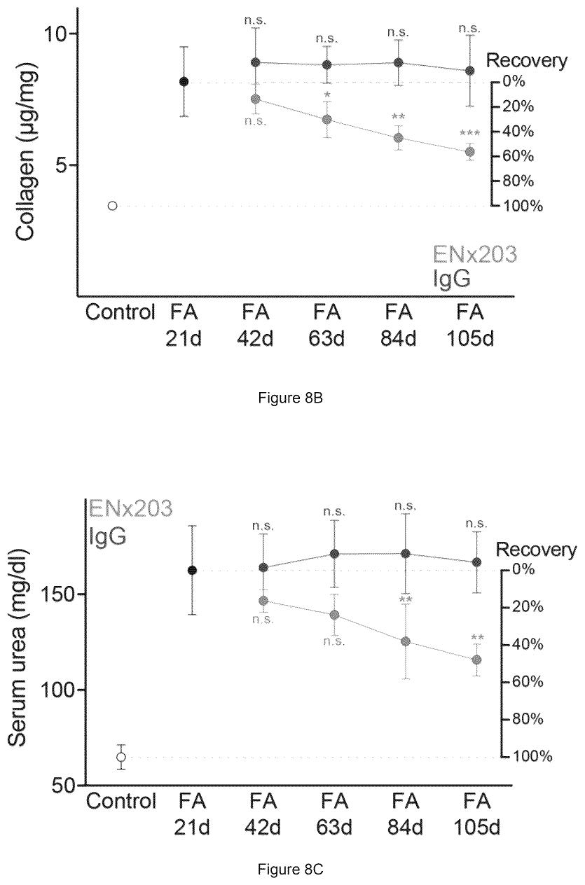

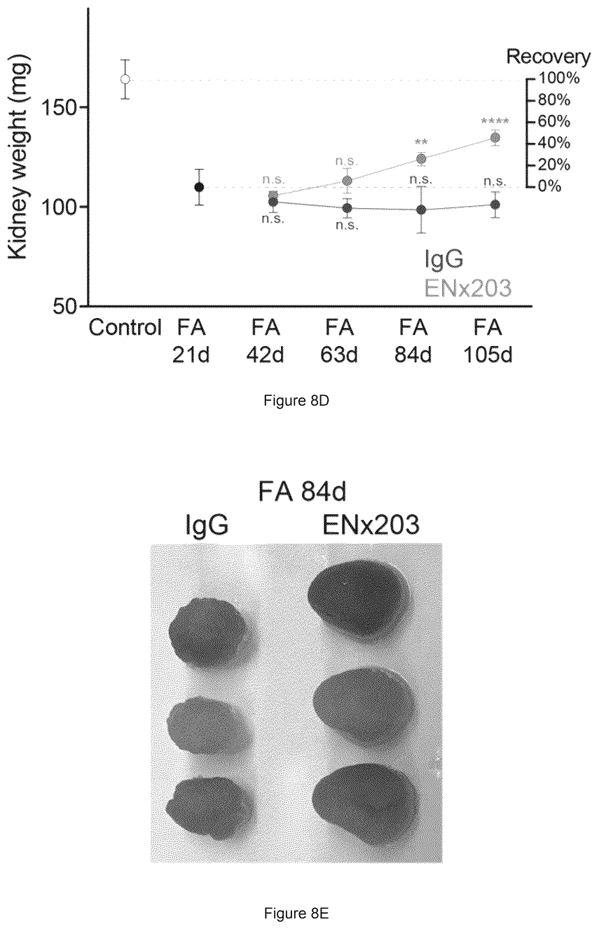

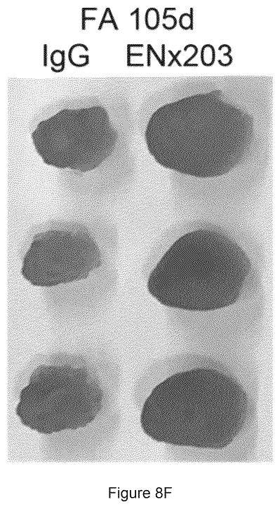

The inventors performed further experiments, in a chemically-induced model of chronic kidney injury. Briefly, renal injury was induced in mice by folate treatment as described above, and were untreated for 21 days. At 21 days post-induction of acute kidney injury by folate treatment, chronic kidney disease had been established, as determined by a ˜2.5 fold increase in blood urea nitrogen (FIG. 8B), and a ˜2.9 fold increase in the albumin to creatine ratio (ACR) in urine (FIG. 11, lower panel). The kidneys had lost ˜33% of their initial mass (FIG. 8C) and collagen levels were elevated ˜2.8-fold (FIG. 8A).

Mice were treated from day 21 with Enx203 or IgG control, at 10 mg/kg, biweekly. Mice were euthanised at the indicated day of the experiment, and renal collagen, serum urea levels, kidney weight and gross kidney morphology were evaluated. The results are shown in FIG. 8. Treatment with Enx203 was associated with reduced levels of renal collagen (FIG. 8A) and reduced levels of serum urea, with an overall 51% reversal in BUN by week 12 of therapy (P<0.001 vs IgG; FIG. 8B).

Importantly, kidney weight increased in the Enx203-treated animals over time, whilst renal collagen content decreased (FIGS. 8C and 8A), and serum urea levels decreased over time in Enx203-treated animals (FIG. 8B). These results indicate that more than inhibiting folate-induced renal tissue injury responses, Enx203 treatment promoted regeneration of functional kidney tissue, reversing the injury phenotype. The kidneys of Enx203-treated mice harvested at days 84 and 105 also more closely resembled healthy kidneys (FIG. 8D and FIG. 8E).

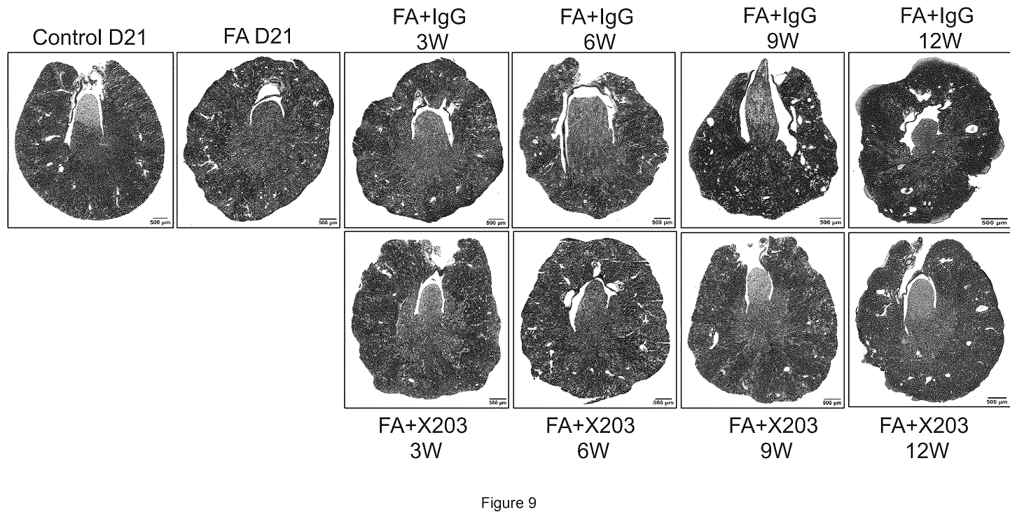

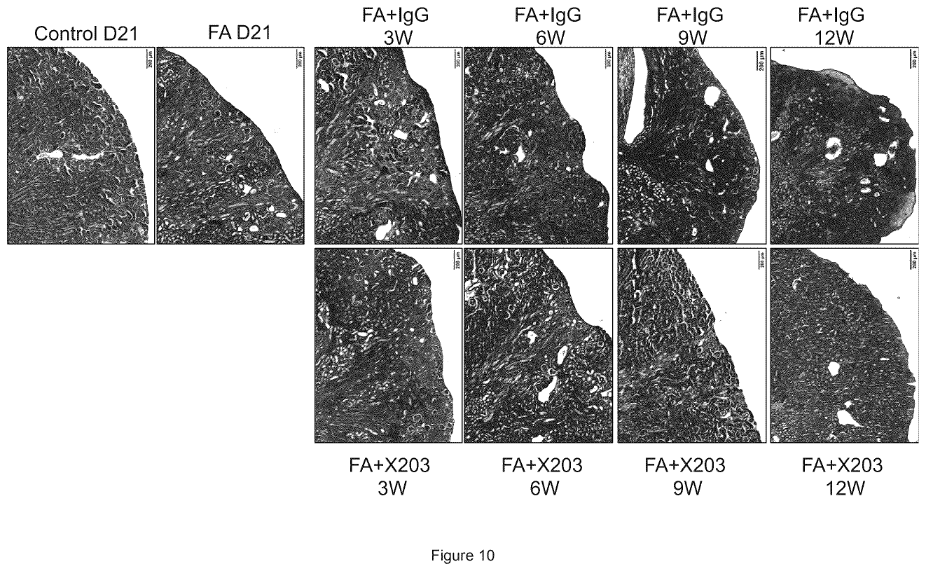

Histological analysis of renal sections from the mice revealed that Enx203 treatment restored normal renal parenchyma (54% reversal in parenchymal loss by week 12 (P<0.001), cortical thickness and volume and cortex morphology in the kidneys of mice subjected to folate-induced kidney injury (FIGS. 9 and 10).

The body weights of mice in this experiment were also monitored throughout the course of the experiment, and Enx203 treatment was found to be associated with weight gain of folate treated mice towards control, untreated levels (FIG. 11, upper panel). The ACR in urine was also monitored in mice at days 21, 84 and 105 of the experiment, and the results are shown in FIG. 11 (lower panel). Treatment with Enx203 was found to cause a substantial reduction in urinary ACR (˜50% reduction within 12 weeks) in folate-treated mice.

The results demonstrated that antagonism of IL-11-mediated signalling could reverse renal fibrosis and produce extensive regeneration of the kidney parenchyma, which is associated with striking reversal of renal impairment.

### Example 3: Investigation of the Molecular Basis of the Inhibition/Reversal of Renal Injury by Antagonists of IL-11 Mediated Signalling

The inventors undertook further investigations to evaluate the role of IL-11 mediated signalling in renal tissue function and injury responses.

The transition of tubule epithelial cells to a mesenchymal cell-like phenotype is implicated in damage to the kidney parenchyma and dysfunction associated with renal injury (see e.g. Lovisa et al. Nat. Med. (2015) 21, 998-1009), and so the inventors investigated whether IL-11 mediated signalling has a role in the epithelial-to-mesenchymal transition for TECs.

Human primary tubule epithelial cells (TECs) were stimulated in vitro with TGFB1, IL-11 (5 ng/ml, 24 h) or TGFB1+Enx203 (2 μg/ml, 24 h), and the percentage of ACTA2^{+ve} cells and collagen expression was evaluated using the Operetta high-content imaging platform.

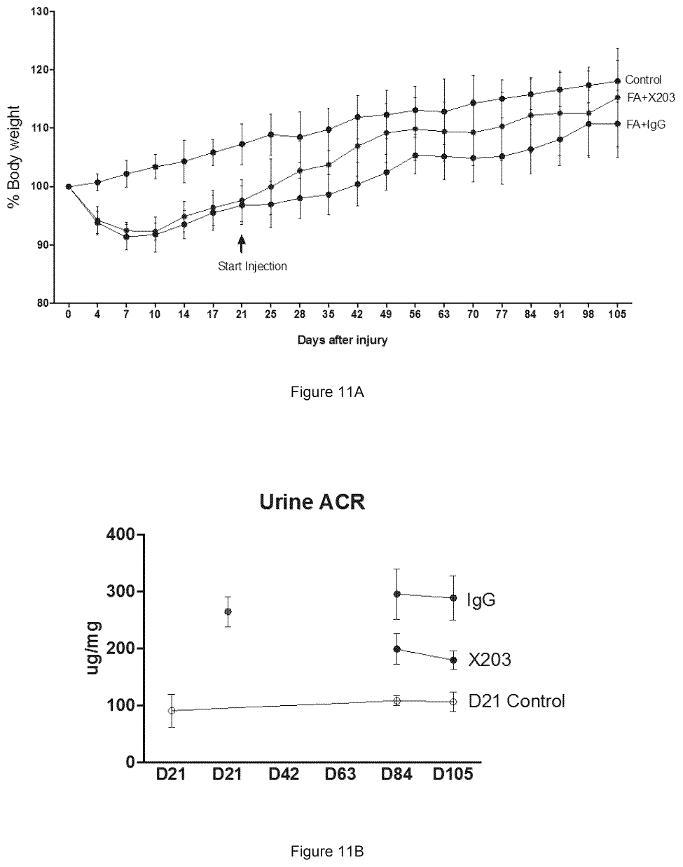

The results are shown in FIG. 12. Both of TGFB1 and IL-11 were found to increase the proportion of ACTA2-expressing TECs, and to increase collagen I expression by these cells. Treatment with Enx203 was found to reduce ACTA2 and collagen I expression by TECs in response to TGFB1/IL-11.

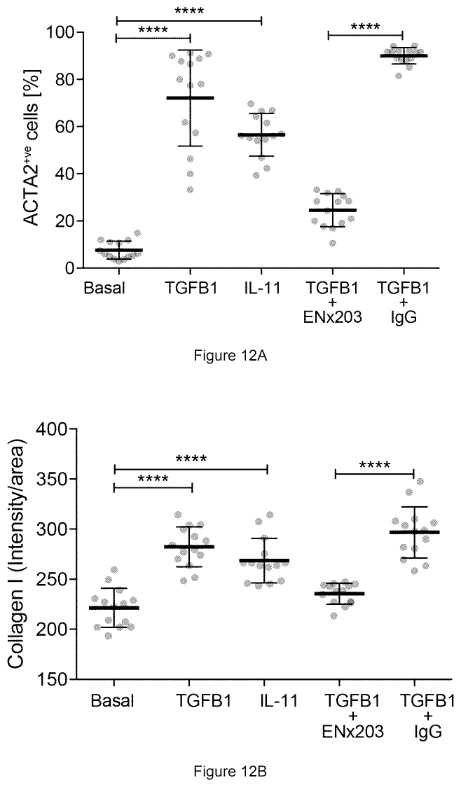

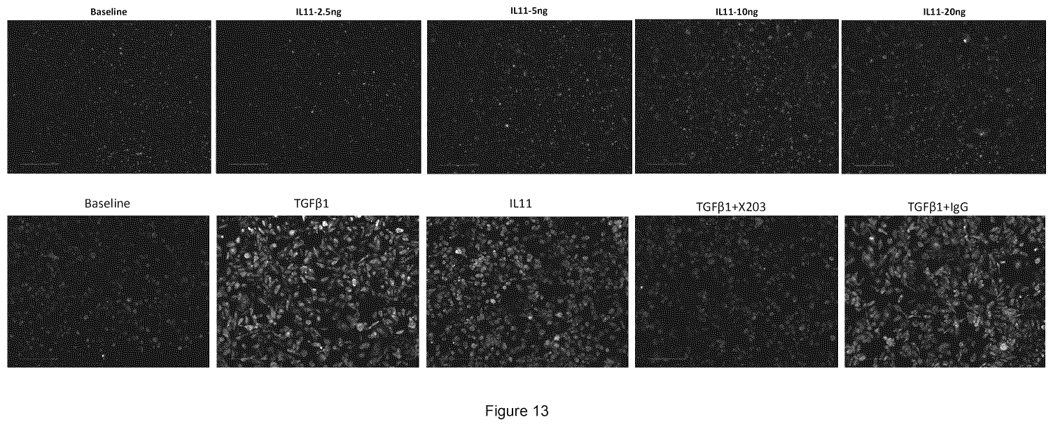

In separate experiments, human primary TECs were stimulated in vitro for 24 hours with different concentrations of IL-11 (2.5 ng/ml, 5 ng/ml, 10 ng/ml), TGFB1 (5 ng/ml), or TGFB1 (5 ng/ml)+Enx203 (2 ug/ml), and Operetta high-content imaging was used to visualise actin stress fibre formation using rhodamine-phalloidin (FIG. 13, upper panels) and collagen (FIG. 13, lower panels).

As explained hereinabove, SNAIL has recently been shown to be a critical determinant of TEC dysfunction following acute kidney injury. Its expression in TECs is associated with impaired TEC function and proliferation. Human primary TECs stimulated in vitro for 24 hours with different concentrations of IL-11 (5 ng/ml), TGFB1 (5 ng/ml), TGFB1 (5 ng/ml)+Enx203 (2 ug/ml) or TGFB1 (5 ng/ml)+IgG control (2 ug/ml) were analysed using the Operetta high-content imaging system for expression of ACTA2 or SNAIL.

The results are shown in FIG. 14. Treatment with IL-11 or TGFB1 was found to induce partial epithelial cell-mesenchymal cell transition (EMT), and expression of ACTA2 and SNAIL. However, the presence of Enx203 strongly inhibited induction of ACTA2 and SNAIL.

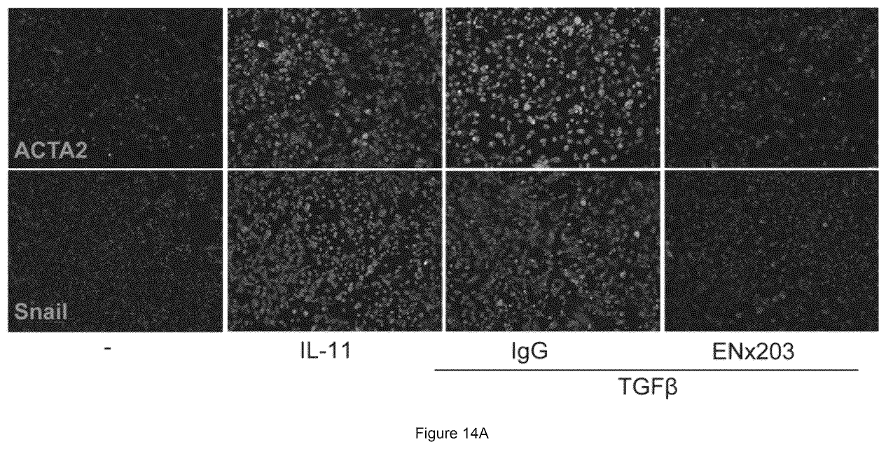

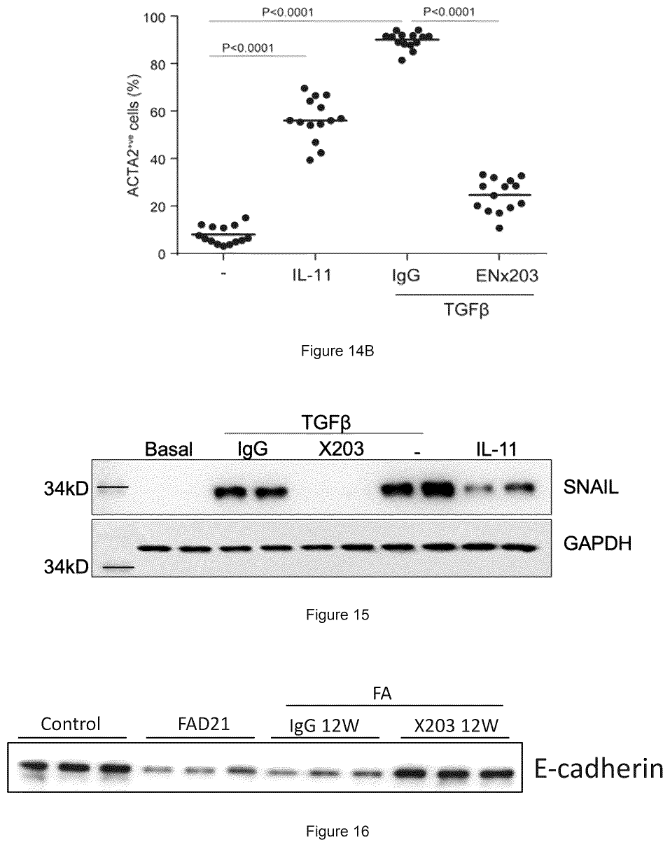

Cell lysates were prepared from the cells, and analysed by western blot for SNAIL expression. A separate western blot for GAPDH was performed as a protein loading control. The results are shown in FIG. 15, and demonstrate that SNAIL expression is upregulated in response to TGFB1 and IL-11, and that TGFB-mediated upregulation of SNAIL was completely prevented by the presence of Enx203.

Thus TGFB1-mediated induction of SNAIL was found to be IL-11-dependent.

The inventors investigated the expression of E-cadherin, the canonical marker of epithelial cell identity, by Western blots of proteins lysates from kidney tissue of mice from the experiment described above in which renal injury was induced in mice by folate treatment as described above, and mice were subsequently treated from day 21 with Enx203 or IgG control, at 10 mg/kg, biweekly.

The results are shown in FIG. 16. Folate-induced renal injury was found to reduce E-cadherin expression, whilst treatment with Enx203 was found to restore E-cadherin expression.

### Example 4: Analysis of the Effect of Inhibition of IL-11 Mediated Signalling in a Model of Physically-Induced Kidney Injury

A mouse model of acute renal injury was induced by unilateral ureteric obstruction (UUO). Briefly, mice were treated by sham operation or ureteric obstruction of one ureter.

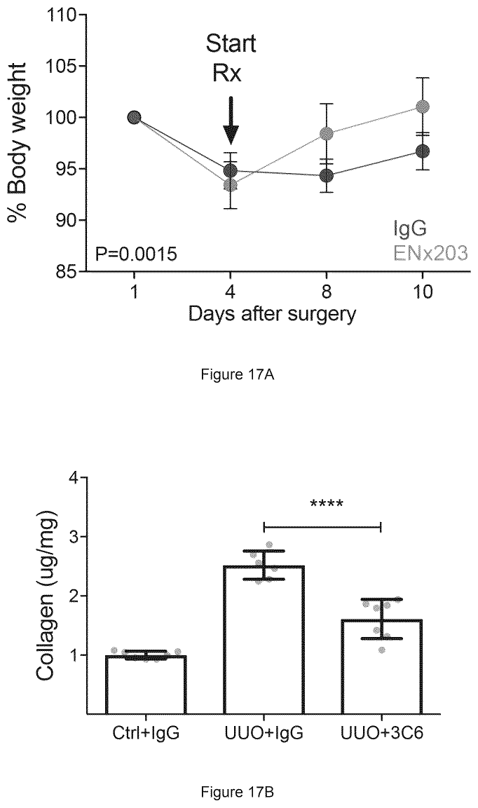

Mice were treated with ENx203 (referred to as ‘3C6’ in FIG. 17B) or IgG control antibody at day 4, 7 and 9 of treatment (20 mg/kg). The body weights of the mice were monitored throughout the experiment, and collagen content in the kidney was assessed at 10 days post UUO surgery using the HPA assay.

The results are shown in FIG. 17. Mice treated with Enx203 had reduced renal collagen, and increased bodyweight as compared to mice subjected to UUO and treated with IgG control antibody.

### Example 5: Analysis of the Effect of Inhibition of IL-11 Mediated Signalling in a Model of Cisplatin-Induced Kidney Injury

Kidney injury was induced in 10 week old C57Bl/6J mice by administration of 7 mg/kg cisplatin, once weekly for four consecutive weeks. A control group was not administered with cisplatin.

Mice were administered biweekly from week 1 by IP injection with X203 (antibody capable of binding to mouse IL-11 (and human IL-11) and inhibiting IL-11 mediated signalling) or an isotype-matched IgG control antibody at a dose of 10 mg/kg, or with saline (control). Mice were harvested for analysis after 8 weeks.

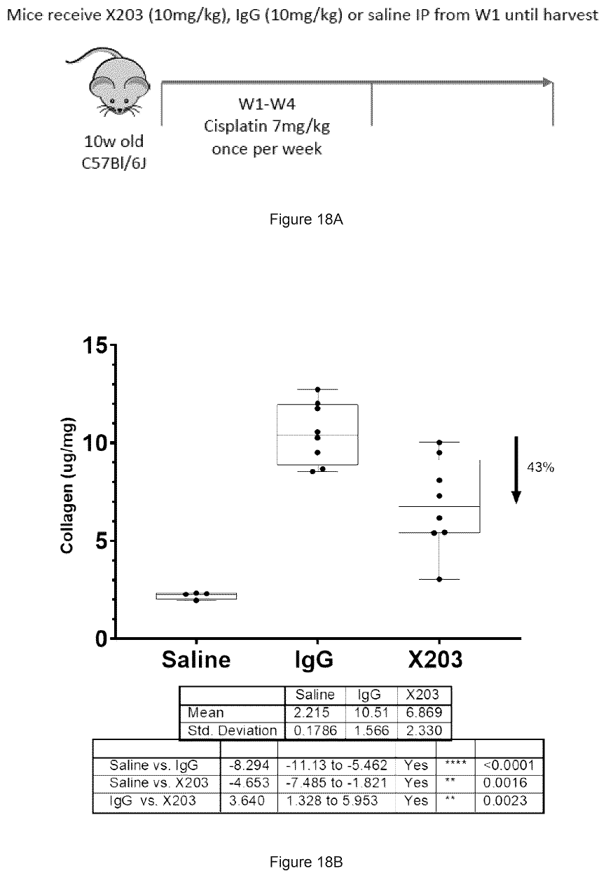

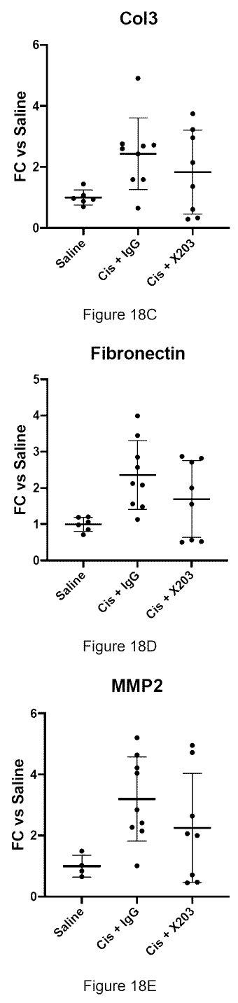

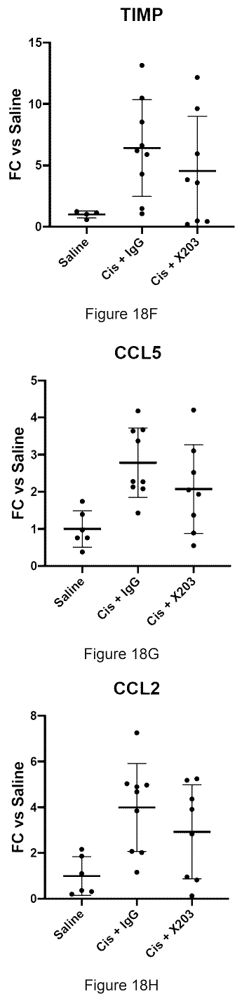

The amount of total collagen in the kidney was quantified on the basis of colourimetric detection of hydroxyproline using a Quickzyme Total Collagen assay kit (Quickzyme Biosciences). The results are shown in FIG. 18B. Cisplatin treatment was associated with increased collagen content of the kidney. Kidneys from cisplatin-treated mice administered neutralising anti-IL-11 antibody had lower collagen content than kidneys from cisplatin-treated mice administered with IgG control antibody. The kidneys of mice subjected to different treatments were also analysed for RNA expression of Col3, Fibronectin, MMP2, TIMP, CCL5 and CCL2 by qPCR.

Briefly, total RNA was extracted from snap-frozen kidney tissue using Trizol (Invitrogen) and RNeasy Mini Kit (Qiagen). PCR amplifications were performed using IScript cDNA Synthesis Kit (Biorad). Gene expression was analyzed in duplicate by TaqMan (Applied Biosystems) or SYBR green (Qiagen) technology using StepOnePlus (Applied Biosystem) over 40 cycles. Expression data were normalized to GAPDH mRNA expression and fold change was calculated relative to expression in saline-treated control subjects.

The results are shown in FIGS. 18C to 18H. Cisplatin treatment was associated with increased expression of Col3, Fibronectin, MMP2, TIMP, CCL5 and CCL2. Kidneys from cisplatin-treated mice administered neutralising anti-IL-11 antibody tended to have lower expression of Col3, Fibronectin, MMP2, TIMP, CCL5 and CCL2 as compared to kidneys from cisplatin-treated mice administered with IgG control antibody.

1. A method of inhibiting the transition of tubular epithelial cells (TECs) to a mesenchymal cell-like phenotype in a subject, comprising administering to a subject in need thereof an effective amount of an anti-interleukin 11 receptor α (IL-11Rα) antibody or an antigen-binding fragment thereof which is an antagonist of IL-11-mediated signaling, thereby inhibiting the transition of TECs to a mesenchymal cell-like phenotype in the subject.

2. The method of claim 1, wherein the subject is a subject suffering from impairment to renal function.

3. The method of claim 1, wherein the subject is a subject suffering from kidney injury, or a disease or condition associated with kidney injury.

4. The method of claim 1, wherein the subject is a subject suffering from a disease or condition characterised by damage to TECs.

5. The method of claim 1, wherein the subject is a subject suffering from a disease or condition selected from: acute kidney injury, acute kidney failure, acute kidney disease, chronic kidney disease, kidney damage, drug-induced kidney injury, ischemia-induced kidney injury, tubular necrosis, acute tubular necrosis, autoimmune kidney injury and cancer.

6. The method of claim 1, wherein the anti-IL-11Rα antibody or antigen-binding fragment thereof comprises:
(i) a heavy chain variable (VH) region incorporating the following CDRs:
HC-CDR1 having the amino acid sequence of SEQ ID NO:46
HC-CDR2 having the amino acid sequence of SEQ ID NO:47
HC-CDR3 having the amino acid sequence of SEQ ID NO:48; and
(ii) a light chain variable (VL) region incorporating the following CDRs:
LC-CDR1 having the amino acid sequence of SEQ ID NO:49
LC-CDR2 having the amino acid sequence of SEQ ID NO:50
LC-CDR3 having the amino acid sequence of SEQ ID NO:51.

7. The method of claim 1, wherein the subject is a subject in which expression of IL-11 or a receptor for IL-11 is upregulated.

8. A method of inhibiting the transition of tubular epithelial cells (TECs) to a mesenchymal cell-like phenotype, comprising contacting TECs with an anti-interleukin 11 receptor α (IL-11Rα) antibody or an antigen-binding fragment thereof which is an antagonist of IL-11-mediated signaling, thereby inhibiting the transition of the TECs to a mesenchymal cell-like phenotype.

9. The method of claim 8, wherein the anti-IL-11Rα antibody or antigen-binding fragment thereof comprises:
(i) a heavy chain variable (VH) region incorporating the following CDRs:
HC-CDR1 having the amino acid sequence of SEQ ID NO:46
HC-CDR2 having the amino acid sequence of SEQ ID NO:47
HC-CDR3 having the amino acid sequence of SEQ ID NO:48; and
(ii) a light chain variable (VL) region incorporating the following CDRs:
LC-CDR1 having the amino acid sequence of SEQ ID NO:49
LC-CDR2 having the amino acid sequence of SEQ ID NO:50
LC-CDR3 having the amino acid sequence of SEQ ID NO:51.
